(19) United States
(12) Patent Application Publication
(10) Pub. No.: US 2022/0252594 A1
(43) Pub. Date: Aug. 11, 2022

(54) GAMMA-DELTA T CELL LIGANDS FOR CANCER IMMUNOTHERAPY

(71) Applicant: The University of Vermont and State Agricultural College, Burlington, VT (US)

(72) Inventors: Ralph C. Budd, Hinesburg, VT (US); Cheryl Collins, Burlington, VT (US)

(73) Assignee: The University of Vermont and State Agricultural College, Burlington, VT (US)

(21) Appl. No.: 17/613,568

(22) PCT Filed: May 27, 2020
(86) PCT No.: PCT/US20/34678

§ 371 Date: Nov. 23, 2021

Related U.S. Application Data
(60) Provisional application No. 62/853,314, filed on May 28, 2019.
(60) Provisional application No. 62/879,999, filed on Jul. 29, 2019.

(51) Int. Cl. G01N 33/566; C07K 14/725

(52) U.S. Cl. CPC G01N 33/566; C07K 14/7051; A61K 39/00; G01N 2500/20; G01N 2333/7051

(57) ABSTRACT

Provided herein are compositions and methods relating to γδ T cells. Specifically, methods of screening samples for human γδ T cells surface ligands using a soluble human γδ T cell receptor tetramer are provided. Such ligands may be useful in the treatment of diseases, for example, cancer.

### RELATED APPLICATIONS

This application claims the benefit under 35 U.S.C. § 119(e) of U.S. provisional application No. 62/853,314, filed May 28, 2019, and U.S. provisional application No. 62/879,999, filed Jul. 29, 2019, the entire disclosure of each of which is incorporated herein by reference in its entirety.

### FEDERALLY SPONSORED RESEARCH

This invention was made with government support under R01 Grant AR43520, R21, Grant AI 107298, and P30 Grant GM118228, awarded by the National Institutes of Health. Accordingly, the government has certain rights in the invention.

### BACKGROUND OF THE INVENTION

γδ T cells function at the interface between the innate and adaptive immune systems and have well-demonstrated roles in response to infection, autoimmunity, and tumors. A common characteristic of these seemingly disparate conditions may be cellular stress. Very few verified ligands for γδ T cells have been identified and these have been largely intact self-proteins with no obvious common structure. In addition, no traditional MHC-restricted recognition of ligands has been demonstrated for γδ T cells. Therefore, full understanding of γδ T cell biology has been handicapped by ignorance of the ligands for most TCR-γδ. To date no systematic process has been reported for determining the spectrum of human TCR-γδ ligands.

### SUMMARY OF THE INVENTION

The disclosure, in some aspects, relates to a method of detecting ligands for γδ T cells in vitro, the method comprising contacting a sample with a soluble human γδ T cell receptor (sTCR-γδ) tetramer, wherein the sTCR-γδ produces a detectable signal in response to engagement with a γδ T cell surface ligand and detecting the measurable signal of the sTCR-γδ tetramer, wherein the detectable signal indicates the presence of the γδ T cell surface ligand in the sample.

In some embodiments, the detectable signal is a fluorescent, chemiluminescent, or absorbance signal. In one embodiment, the sTCR-γδ tetramer is biotinylated and the detectable signal is streptavidin-PE. In some embodiments, the staining is detected via flow cytometry.

In some embodiments, the sTCR-γδ binds to the γδ T cell surface ligand of a Vδ1 T cell. In some embodiments, the sample comprises primary cells or a tumor cell line. In some embodiments, the sample is from a primary tissue, a tumor, inflamed synovium, or intestinal epithelium.

In some embodiments, the method further comprises identifying the γδ T cell surface ligand. In some embodiments, the γδ T cell surface ligand is identified using RNA-seq and bioinformatics and/or mass spectrometry, and/or a transfection-based genetic screen.

The disclosure, in another aspect, provides a human synovial soluble TCR-γδ. In some embodiments, the human synovial soluble TCR-γδ is formulated as a tetramer using, for example, streptavidin-PE or avidin-conjugated magnetic beads.

The disclosure, in a further aspect, provides a single vector comprising a T cell receptor (TCR) γ chain sequence and a TCR δ chain sequence, and further comprising two promoters, a tag, and a binding partner sequence. In some embodiments, the tag is a hexa-His tag. In some embodiments, the binding partner sequence is a biotinylation sequence. In some embodiments, the two promoters comprise p10 and polyhedron.

In one embodiment, the disclosure provides a method of making the human synovial soluble TCR-γδ, the method comprising transfecting a cell with a vector described herein.

The disclosure, in another aspect, provides an anti-cancer therapeutic composition, comprising a unique TCR-γδ ligand, wherein the unique TCR-γδ ligand is a protein or a functional fragment thereof of Table 1 and a pharmaceutically acceptable carrier for administration to a subject to stimulate a γδ T cell subpopulation.

In a further aspect, the disclosure provides a method for stimulating a γδ T cell subpopulation in vivo, the method comprising: administering to a subject a unique TCR-γδ ligand, wherein the unique TCR-γδ ligand is a protein or a functional fragment thereof of Table 1 and a pharmaceutically acceptable carrier in an effective amount to stimulate a γδ T cell subpopulation.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Examples, Claims, and Figures. Other features, objects, and advantages of the invention will be apparent from the description and from the claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1F show the production of human synovial soluble TCR-γδ (sTCR-γδ) and crystals. FIG. 1A shows a pBACp10 pH vector containing the δ-chain driven by the polyhedrin promoter, and the γ-chain with hexa-His and biotinylation BRP sequences driven by the p10 promoter from γδ T cell clone Bb15 (Vγ9Vδ1). FIG. 1B shows a sample of nickel NTA column-purified sTCR-γδ analyzed by SDS-PAGE under reducing and non-reducing conditions, and stained with Coomassie Blue. FIG. 1C is an immunoblot of sTCR-γδ stained with anti-Vδ1 or anti-Cγ. FIG. 1D shows a γδ T cell clone Bb15 stained with anti-TCR-γδ antibody in the absence or presence of competing sTCR-γδ. FIG. 1E is a graph showing the fibrosarcoma cell line 2fTGH stained with the sTCR-γδ in the absence or presence of the indicated concentrations of anti-γδ antibody or control IgG. FIG. 1F shows a titration of sTCR-γδ staining of the positively staining tumor line 2fTGH or negatively staining line Daudi. Number inserts indicate percent positively staining cells. Findings are representative of three experiments.

FIGS. 2A-2B show a sTCR-γδ tetramer staining of a tumor panel. A panel of 24 diverse tumor cell types was stained with either sTCR-αβ or sTCR-γδ. Shown are examples of tumors representing either positive staining (FIG. 2A) or negative staining with sTCR-γδ (FIG. 2B), with the complete list summarized below each example. Number inserts indicate mean fluorescence intensity of entire histogram. Findings are representative of four experiments.

FIGS. 3A-3D show that a sTCR-γδ ligand(s) is sensitive to protease, blockers of ER-Golgi transport, translation, or transcription, and contain glycosaminoglycans (GAGs). The human bronchoepithelial cell line was either untreated or treated with trypsin for 15 minutes (FIG. 3A), or untreated or treated for 18 hours with cycloheximide or actinomycin D (FIG. 3B), or untreated or treated for 18 hours with Brefeldin A or Monensin (FIG. 3C). Cells were then stained with sTCR-γδ tetramer. FIG. 3D shows that the 2fTGH fibrosarcoma cell line, wild-type CHO cells, or GAG-deficient CHO cells were either untreated or treated with a combination of heparinases I-III for 30 minutes and then stained with sTCR-γδ tetramer. Number inserts indicate mean fluorescence intensity of entire histogram. Findings are representative of three experiments.

FIGS. 4A-4C show that TCR-γδ ligand is induced on human monocytes following activation. FIG. 4A shows a flow cytometric analysis. Freshly isolated monocytes were either unstimulated or activated with Borrelia burgdorferi or LPS for 18 hours and then stained with the indicated reagents. In another study, fresh monocytes or monocytes activated with Borrelia were incubated in the presence of medium alone or TNFα or blocking anti-TNFα (FIG. 4B), or IL-1β or blocking anti-IL-1β (FIG. 4C). Number inserts indicate percent positively staining cells. Error bars represent SEM. Findings are representative of four experiments.

FIGS. 5A-5C show that sTCR-γδ tetramer stains a subset of activated human T cells and Treg. PBL were stained with antibodies to CD4 and CD8 as well as with sTCR-αβ tetramer-PE or sTCR-γδ tetramer-PE either freshly isolated (FIG. 5A), or 3 days after activation with anti-CD3/CD28+IL-2 (FIG. 5B). Number inserts indicate the percentages of T cells staining negatively or positively with sTCR-γδ tetramer, as a portion of the total CD4⁺ or CD8⁺ subsets, as well as mean fluorescence intensity (MFI) in some cases. Findings are representative of six experiments. FIG. 5C depicts freshly isolated PBL stained with anti-CD4, anti-CD25 or isotype control, and streptavidin-PE (SA-PE) or sTCR-γδ-PE. Shown are cells gated on CD4 expression. Number inserts indicate mean fluorescence intensity (MFI) of sTCR-γδ-PE staining for CD25⁺ and CD25⁻ subsets. Findings are representative of two experiments.

FIGS. 6A-6C show that TCR-γδ ligand expression parallels glycolysis. FIGS. 6A and 6B show that PBL were activated with anti-CD3/CD28+IL-2 in the absence or presence of 2-deoxyglucose (2-DG, 5 mM). On day 3 cells were stained with antibodies to CD4, CD8, CD25, and sTCR-γδ tetramer-PE. FIG. 6A shows the levels of CD25 and TCR-γδ ligand without or with 2-DG. FIG. 6B shows the expression of TCR-γδ ligand in CD4⁺ or CD8⁺ subsets based on surface CD25. FIG. 6C illustrates that after 2fTGH cells were cultured for 48 hours in either regular medium or medium plus 2-DG (5 mM), cells were stained with TCR-αβ or TCR-γδ. Number inserts indicate mean fluorescence intensity (MFI) of sTCR-γδ-PE staining. Findings are representative of three experiments.

### DETAILED DESCRIPTION OF THE INVENTION

Described herein are methods of identifying candidate ligands for human γδ T cells using a soluble human TCR-γδ molecule and related products.

A detectable form of human soluble TCR-γδ (sTCR-γδ) was produced from a synovial Vδ1 γδ T cell clone of a Lyme arthritis patient. As described herein, the tetramerized sTCR-γδ was used in flow cytometry to identify various cell types that expressed candidate ligands. Initial analysis of 24 tumor cell lines identified a set of 8 ligand-positive tumors, enriched for those of epithelial and fibroblast origin, and 16 ligand-negative tumors, largely of hematopoietic origin. In addition, ligand was not expressed by primary monocytes or T cells, although each could be induced to express ligand following their activation. Ligand expression was sensitive to trypsin digestion, revealing the protein nature of the ligands, and was also reduced by inhibition of glycolysis. These findings provide a framework and strategy for the identification of individual ligands for human synovial γδ T cells.

γδ T cells reside at mucosal and epithelial barriers, and often accumulate at sites of inflammation with autoimmunity, infections, or tumors (1). Evidence suggests that γδ T cells provide protection against infections with bacteria, viruses, and protozoans, and are generally beneficial in autoimmunity (1-17). In addition, a role for γδ T cells in the immune response against tumors in humans is evident from a seminal study reporting that intratumoral γδ T cells are the most favorable prognostic immune population across 39 cancer types in humans (18). γδ T cells are often highly lytic against transformed proliferative cells, infected cells, as well as infiltrating CD4⁺ T cells in inflammatory arthritis (9, 17, 19). They can produce a variety of cytokines including IFN-γ, TNF-α, and IL-17 (20), as well as insulin-like growth factor-1 (IGF1) and keratinocyte growth factor (KGF) that promote epithelial wound repair (21). These collective studies indicate that a principal function of γδ T cells is in response to tissue injury of various causes. It is, thus, not surprising that γδ T cells are often suggested to react to host components that are upregulated or exposed during proliferation or cell injury (22). As such, γδ T cells may function in tissue homeostasis and immunoregulation as much as in protection from infection. Yet in the vast majority of cases, little if anything is known regarding the nature of these self-components, or whether they actually engage the TCR-78.

Whereas αβ T cells recognize proteins that are processed into peptides and presented on MHC molecules, the few proposed ligands for γδ T cells suggest that they recognize mostly intact proteins directly, without MHC restriction. This makes them highly attractive for immunotherapy. Despite the elaborate mechanisms that αβ T cells and B cells use to prevent autoreactivity, γδ T cells have been frequently reported to respond to autologous proteins. Furthermore, in contrast to other lymphocytes that maximize the potential diversity of their receptors, γδ T cells frequently show limitations in their diversity. Thus, human γδ T cells comprise subset of Vδ2 T cells, the predominant γδ in peripheral blood that respond to prenyl phosphates and certain alkyl amines (23-25), and Vδ1 T cells that do not respond to these compounds and often accumulate at epithelial barriers and sites of inflammation (1). A similar limited repertoire occurs in the mouse in which Vγ5Vδ1 cells colonize the epidermis, and a Vγ6Vδ1 subset colonizes the tongue, lung, and female reproductive tract (21, 26). This restricted repertoire implies that TCR-γδ ligands may also be limited. This may provide for a more rapid response, and perhaps explain why, in contrast to αβ T cells and B cells, it is difficult to generate antigen-specific γδ T cells by immunization with a defined antigen.

Various ligands for γδ T cells have been proposed, although only a few have been confirmed to bind to TCR-γδ, and these lack any obvious similarity in structure. γδ T cells for which ligands have been identified include the murine γδ T cell clone G8, which recognizes the MHC class I-like molecules T10 and T22 (27), γδ T cells from mice infected with herpes simplex virus that recognize herpes glycoprotein gL (28), a subset of murine and human γδ T cells that bind the algae protein phycoerythrin (20), a human γδ T cell clone G115 that recognizes ATP synthase complexed with ApoA-1 (28), a human γδ T cell clone (Vγ4Vδ5) from a CMV-infected transplant patient that recognizes endothelial protein C receptor (EPCR) (29), and some human Vδ1 T cells that recognize CD1d-sulfatide antigens (30). However, to date no systematic process has been reported for determining the spectrum of human TCR-γδ ligands.

Therefore, in some aspects, the disclosure provides a method of systemically identifying human TCR-γδ ligands, such as those that interact with Vδ1 γδ T cells. In some embodiments, the human TCR-γδ ligands are identified with the use of a soluble TCR-γδ tetramer linked directly or indirectly to a detectable molecule.

As used herein, a “soluble TCR-γδ” refers to a T cell receptor consisting of the chains of a full-length (e.g., membrane bound) receptor, except that, minimally, the transmembrane regions of the receptor chains are deleted or mutated so that the receptor, when expressed by a cell, will not associate with the membrane. Most typically, a soluble receptor will consist of only the extracellular domains of the chains of the wild-type receptor (i.e., lacks the transmembrane and cytoplasmic domains). TCR-γδ molecules comprise a heterodimer of a γ chain and a δ chain. Multiple different functional murine γ chains, murine δ chains, human γ chains, and human δ chains are known in the art. Various specific combinations of γ and δ chains are preferred for use in the sTCR-γδs described herein, and particularly those corresponding to γδ T cell subsets that are known to exist in vivo, but it is to be understood that sTCR-γδs having virtually any combination of γ and δ chains are also contemplated for use herein. Preferably, sTCR-γδs comprise γ and δ chains derived from the same animal species (e.g., murine, human). In some embodiments, the sTCR-γδ comprises human γ chains and human δ chains. A sTCR-γδ described herein may comprise a heterodimer comprising a γ chain and a δ chain. In some embodiments, the sTCR-γδ described herein is a multimer (e.g., tetramer) comprising four of the same γδ heterodimers. In some embodiments, the sTCR-γδ comprises 2, 3, 4, 5, 6, 7, 8, 9, 10, or more γδ heterodimers. As described above, in some embodiments, γ and δ chains from the same species of mammal (e.g., murine, human) are combined to form a γδ heterodimer.

The heterodimers may be linked or conjugated by any method known in the art, for example, by streptavidin tetramerization. In one embodiment, the heterodimers are linked via a linker radical comprising a polyalkylene glycol polymer or a peptidic sequence. In some embodiments, the linker radical should be capable of attachment to defined positions on the sTCR-γδs, so that the structural diversity of the multimers formed is minimized. In one embodiment, the polymer chain or peptidic linker sequence extends between amino acid residues of each sTCR-γδ which are not located in a variable region sequence of the sTCR-γδ thereof.

Methods of linking the heterodimer chains are known in the art, and two examples are provided below. In some embodiments, the mulitmer (e.g., tetramer) described herein is linked by a polyalkylene glycol chain. In some embodiments, the polyalkylene glycol chain comprises hydrophilic polymers. Examples of polyalkylene glycols include, but are not limited to those based on polyethylene glycol or PEG, as well as those based on other suitable, optionally substituted, polyalkylene glycols, such as polypropylene glycol, and copolymers of ethylene glycol and propylene glycol. In other embodiments, the multimer (e.g., tetramer) is multimerized using a non-PEG-based polymer, such as moieties comprising maleimide termini linked by aliphatic chains such as BMH and BMOE can be used.

In some embodiments, the multimerization is accomplished through the use of one or more peptidic linkers. These linkers are comprised of chains of amino acids, and function to produce simple linkers or multimerization domains onto which sTCR-γδs can be attached. As noted above, the biotin/streptavidin system has previously been used to produce tetramers of murine TCR-γδs (see WO 99/60119) for in vitro binding studies.

There are a number of human proteins that contain a multimerization domain that could be used in the production of sTCR-γδs. For example, the tetramerization domain of p53 which has been utilized to produce tetramers of scFv antibody fragments which exhibited increased serum persistence and significantly reduced off-rate compared to the monomeric scFV fragment may be used. (Willuda et al. (2001) J. Biol. Chem. 276 (17) 14385-14392) Likewise, hemoglobin also has a tetramerization domain that could potentially be used.

A multimer (e.g., tetramer) complex comprising at least two sTCR-γδs wherein at least one of said sTCR-γδs is a sTCR-γδ described herein provides another embodiment of the disclosure.

The sTCR-γδ produces a detectable signal in response to engagement with a γδ T cell surface ligand. A detectable signal may be produced once a ligand interacts with sTCR-γδ and induces a change that enables detection of a signal. The signal may be in the form of a detectable molecule.

The detectable molecule may be any agent known in the art, for example, an agent capable of generating a fluorescent, chemiluminescent, or absorbance signal. A suitable label may be chosen from a variety of known detectable labels. Exemplary labels include fluorescent, photoactivatable, enzymatic, epitope, magnetic and particle (e.g. gold) labels. In some embodiments, the detectable molecule comprises one or more fluorescent labels, such as FITC. For example, in tetrameric sTCR-γδ formed using biotinylated heterodimers, fluorescent streptavidin (commercially available) can be used to provide a detectable label. A fluorescently labeled tetramer will be suitable for use in FACS analysis, for example to detect one or more γδ T cell ligands.

In some embodiments, the detectable agent is directly conjugated to the sTCR-γδ. In other embodiments, the detectable agent is indirectly conjugated to the sTCR-γδ. For example, the sTCR-γδ may be labeled either directly with a fluorescent tag, or with a hapten such as biotin, followed by treatment with a fluorescently labeled second moiety such as streptavidin (or both). The latter technique may be particularly advantageous to “amplify” the fluorogenicity of the target (sTCR-γδ), thus allowing smaller amounts of target to be used and/or detected. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. In addition, suitable optical dyes are described in the 1996 Molecular Probes Handbook by Richard P. Haugland.

In one embodiment, the fluorescent label is functionalized to facilitate covalent attachment of the label to the sTCR-γδ. A wide variety of fluorescent labels are commercially available which contain functional groups, including, but not limited to, isothiocyanate groups, amino groups, haloacetyl groups, maleimides, succinimidyl esters, and sulfonyl halides, all of which may be used to covalently attach the fluorescent label to the sTCR-γδ. The choice of the functional group of the fluorescent label will depend on the site of attachment to either a linker, as described below, or directly to the sTCR-γδ.

The covalent attachment of the fluorescent label may be either direct or via a linker. In one embodiment, the linker is a relatively short coupling moiety. A coupling moiety may be synthesized directly onto a sTCR-γδ molecule, for example, and may contain at least one functional group to facilitate attachment of the fluorescent label. Alternatively, the coupling moiety may have at least two functional groups, which are used to attach a functionalized candidate agent to a functionalized fluorescent label, for example. In an additional embodiment, the linker is a polymer. In this embodiment, covalent attachment is accomplished either directly, or through the use of coupling moieties from the agent or label to the polymer. In some embodiments, the covalent attachment is direct, that is, no linker is used. In this embodiment, the candidate agent preferably contains a functional group, such as a carboxylic acid, which is used for direct attachment to the functionalized fluorescent label. Thus, for example, for direct linkage to a carboxylic acid group of a candidate agent, amino modified or hydrazine modified fluorescent labels will be used for coupling via carbodiimide chemistry, for example using 1-ethyl-3-(3-dimethylaminopropyl)-carbodiimide (EDC) as is known in the art (see Set 9 and Set 11 of the Molecular Probes Catalog, supra; see also the Pierce 1994 Catalog and Handbook, pages T-155 to T-200). In one embodiment, the carbodiimide is first attached to the fluorescent label, such as is commercially available.

In other embodiments, the labeling may be accomplished through the use of a binding pair, that is, a first binding moiety directly attached to the sTCR-γδ, and a second binding moiety comprising a detectable signal (e.g., a fluorescent molecule) and is capable of binding to the binding pair agent attached to the sTCR-γδ.

Suitable binding pairs include, but are not limited to, antigens/antibodies (e.g., anti-γδ TCR antibodies), including digoxigenin/antibody, dinitrophenyl (DNP)/anti-DNP, dansyl-X/anti-dansyl, fluorescein/anti-fluorescein, lucifer yellow/anti-lucifer yellow, rhodamine/anti-rhodamine; and biotin/avidin (or biotin/strepavidin). Preferred binding pairs (i.e., first and second labeling moieties) generally have high affinities for each other, and in some embodiments, are able to withstand the shear forces during FACS sorting.

The measurable/detectable signal may be identified using any method known in the art for the type of detectable signal used. In some embodiments, the analysis is carried out using flow cytometry (FACS). In other embodiments, signal-specific assays, such as ELISAs are used. In other embodiments, fluorescence imaging may be used.

The level of the detectable signal and/or the existence of a detectable signal may indicate the presence of one or more γδ T cell surface ligands. In further embodiments, the one or more γδ T cell surface ligands are identified using any method known in the art. For example, RNA sequencing (whole transcriptome shotgun sequencing, RNAseq), bioinformatics, and/or genetic screening (transfection-based genetic screens) may be used to identify the one or more γδ T cell surface ligands. A non-limiting exemplary list of γδ T cell surface ligands identified using any of the methods disclosed herein is provided in Table 1.

Samples may be screened for the presence of γδ T cell surface ligands. Such samples include, without limitation, plasma, serum, cell, or tissue samples. In some embodiments, a primary tissue sample is used (e.g., tissue from the gut mucosa (intestinal epithelium), synovium, skin, lungs, uterus, etc.). In one embodiment, the sample is from inflamed synovium. In another embodiment, a cell line, such as a tumor cell line is used. Tumor cell lines are known in the art and include, for example, CRF-CEM, HL-60(TB), K-562, MOLT-4, RPMI-8226, SR, A549/ATCC, EKVX, HOP-62, HOP-92, NCI-H226, NCI-H23, NCI-H322M, NCI-H460, NCI-H522, COLO 205, HCC-2998, HCT116, HCT-15, HT-29, KM12, SW-620, SF-268, SF-295, SF-539, SNB-19, SNB-75, U251, LOX IMVI, MALME-3M, M14, MDA-MB-435, SK-MEL-2, SK-MEL-28, SK-MEL-5, UACC-257, UACC-62, IGR-OV1, OVCAR-3, OVCAR-4, OVCAR-5, OVCAR-8, NCI/ADR-RES (previously, MCF-7/ADR-RES), SK-OV-3, 786-O, A498, ACHN, CAKI-1, RXF 393, SN12C, TK-10, UO-31, PC-3, DU-145, MCF7, MDA-MB-231/ATCC, MDA-MB-468, HS 578T, MDA-N, BT-549, T-47D, LXFL 529, DMS 114, SHP-77, DLD-1, KM20L2, SNB-78, XF 498, RPMI-7951, M19-MEL, RXF-631, SN12K1, P388, and P388/ADR. Other tumor lines include 2fTGH, HEK 293 T, Hep3B, HT-29, IMR-90, and TE671. Samples may be obtained by any means known in the art, for example, through commercial sources or through biopsies or blood draws.

The sample, in some embodiments, comes from a subject. A subject shall mean a human or vertebrate animal including but not limited to a dog, cat, horse, cow, pig, sheep, goat, turkey, chicken, primate, e.g., monkey, and fish (aquaculture species), e.g. salmon. In one embodiment, the subject is a human.

Without wishing to be bound by theory, it is thought that the γδ T cell surface ligands identified, for example, using any of the methods disclosed herein, will be useful in a wide variety of applications, such as cancer immunotherapy (Pauza et al., Frontiers in Immunology, 2018, 9(1305):1-11). For example, administration of synthetic γδ T cell surface ligands may activate γδ T cells in vivo, leading to enhanced antitumor effects. Activated γδ T cells, as noted above, produce a variety of chemokines and cytokines, regulate other immune and non-immune cells, and present antigen (e.g., may induce primary CD4+ and CD8+ T cell responses to antigens). The γδ T cells are also able to aid B helper cells and therefore play a regulatory role in humoral immunity. They can also activate immature dendritic cells. Taken together, activating ligands may yield significant immunotherapy benefits.

Therefore, in one embodiment, the disclosure provides an anti-cancer therapeutic composition. The anti-cancer therapeutic composition may comprise a unique TCR-γδ ligand (e.g., a protein or a functional fragment thereof of Table 1) and a pharmaceutically acceptable carrier (excipient) for administration to a subject to stimulate a γδ T cell subpopulation.

“Acceptable” means that the carrier must be compatible with the active ingredient of the composition (and preferably, capable of stabilizing the active ingredient) and not deleterious to the subject to be treated. Pharmaceutically acceptable excipients (carriers) include buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the TCR-γδ ligands as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder, such as a cancer.

A subject suspected of having any of such target disease/disorder (e.g., cancer) might show one or more symptoms of the disease/disorder. A subject at risk for the disease/disorder can be a subject having one or more of the risk factors for that disease/disorder.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1. Detection of Cell Surface Ligands for Human Synovial γδ T Cells

### Materials and Methods

### Production of a Soluble TCR-γδ(sTCR-γδ).

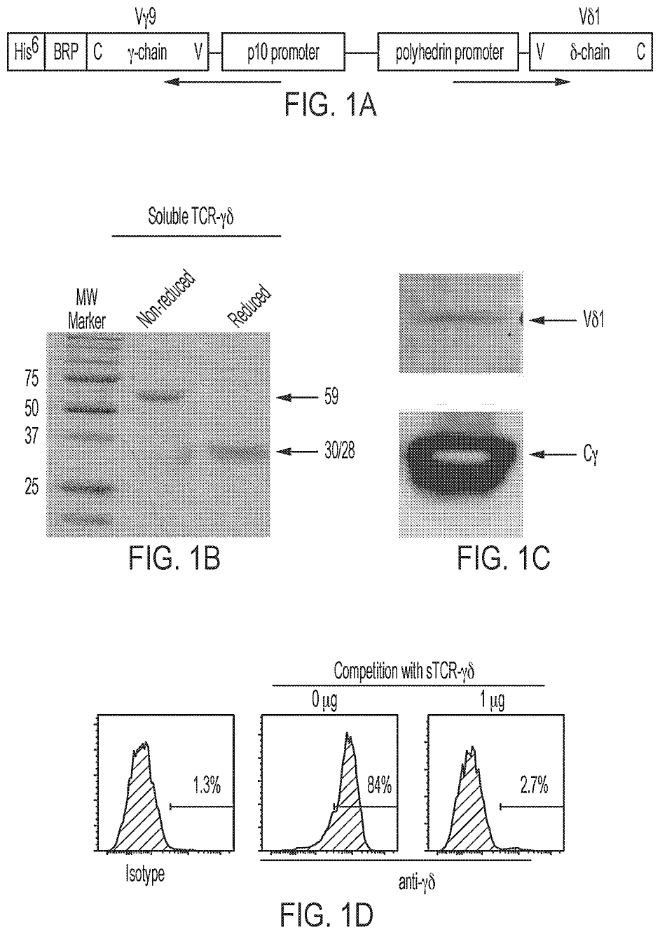

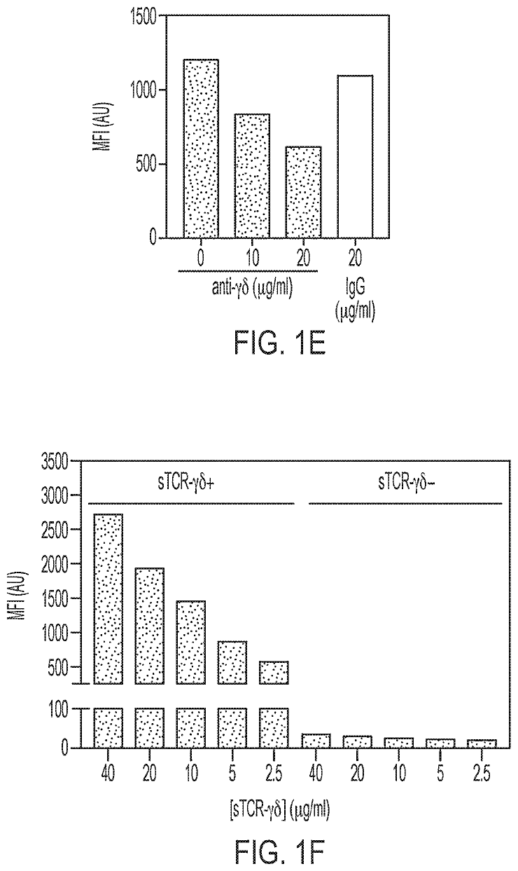

Human synovial γδ T cell clones from a Lyme arthritis patient were produced as previously described (9, 31). One of these clones, Bb15, was chosen for production of the sTCR-γδ using modification of a previously reported procedure (32, 33). Both TCR chains were produced as a single transcript in a baculovirus vector. The pBACp10 pH vector used contains two back-to-back promoters, p10 and polyhedrin (FIG. 1A). The p10 promoter is followed by multiple cloning sites for the γ-chain, and the polyhedrin promoter is followed by multiple cloning sites for the δ-chain. Downstream of the γ-chain a hexa-His tag was placed for nickel column purification, followed by a biotinylation sequence for tetramerization. The γ-chain and δ-chain were PCR amplified using high fidelity polymerase (Deep Vent Polymerase, NEB). Both TCR chain sequences were verified following the initial PCR amplification as well as after insertion into the pBACp10 pH vector. Virus encoding the sTCR-γδ was generated by co-transfection of Sf21 moth cells using the Sapphire baculovirus DNA and Transfection kit (Orbigen) with the sTCR pBACp10 pH construct. Virus was harvested 6 days later and used as primary stocks (P1 stock).

Two additional rounds of viral amplification—P2 and P3—were completed using mid-log phase Sf21 cells (˜1.6×10⁶ cells/mL) allowed to adhere for 1 hour (h) before infecting at a MOI of 0.01 or 0.1 with P1 and P2 stock, respectively. After 72 h of infection, culture media was clarified by centrifugation (1000×g for 10 minutes) and filtration (VacuCap 90PF 0.8/0.2 μm Supor membrane filter units; PALL Corporation, Westborough, Mass.) before being stored in the dark at 4° C. until use. Protein production occurred in 12 L batches of the mid-log phase (˜1.6×10⁶ cells/mL) Hi5 cells were grown in suspension (0.5 L of culture in 1 L spinner flasks) and infected with P3 stock at a 1:50 dilution. Following 72 h of infection, cells were removed by centrifugation and filtration as described above. The filtered supernatant (approximately 12 L) containing secreted sTCR-γδ was concentrated to approximately 100 mL. The supernatant was then dialyzed against 1 L of nickel column loading buffer (20 mM NaPhosphate buffer pH 7.4, 20 mM imidazole, 0.5 M NaCl) using a Pellicon diafiltration system with two 10K MWCO membranes (Millipore, Burlington, Mass.) back down to a volume of approximately 100 mL. After system flushing, the final sample volume was approximately 200 mL. It was then loaded onto loading-buffer-equilibrated His-Trap HP columns (GE Healthcare, Little Chalfont, UK) at 100 mL per 2×5 mL columns. Columns were washed with at least 10 column volumes of loading buffer until baseline absorption was achieved. Bound proteins were eluted using a gradient from 20 mM to 500 mM imidiazole over 20 column volumes. Elution was monitored by absorbance at 280 nM and 1 mL fractions were collected. Fractions containing the target protein were identified using SDS-PAGE gel analysis using Coomassie Blue. High purity (>95%) sTCR-γδ fractions were pooled, dialyzed against PBS pH 7.4, and frozen at −80° C. until used in future studies. Yields were typically approximately 1.0 to 2.5 mg/L of culture.

Purified sTCR-γδ was then biotinylated using a biotin-protein ligase system (Avidity, Inc.) and tetramerized with streptavidin-PE (BioLegend) for FACS staining. Verification of TCR-γδ protein was confirmed by SDS-PAGE gel analysis using Coomassie Blue as well as immunoblot using antibodies to Vδ1 or Cγ (Endogen).

### Purification and Activation of Human Monocytes and T Cells, and Tumor Cell Lines.

Human monocytes were purified from human peripheral blood mononuclear cells (PBMC) using CD14 labeled magnetic beads, followed by column purification (Miltenyi) and then cultured in RPMI complete with 10% FCS in the absence or presence of either a Borrelia burgdorferi sonicate (10 μg/ml) or LPS (1 μg/ml; Sigma) for 18 h. To some cultures, TNFα (10 ng/ml) (Biolegend), anti-TNFα (10 μg/ml) (Biolegend), IL-1β (10 pg/ml) (Invitrogen), or anti-IL-1β (5 μg/ml) (R&D Systems) were added. Cells were then stained with the sTCR-γδ tetramer.

T cells from PBMC were used either fresh or were activated with anti-CD3/anti-CD28 (each 10 μg/ml; BioLegend)+IL-2 (50 U/ml; Cetus) and propagated for three days. Cells were then stained with the sTCR-γδ-tetramer. Tumor cell lines were obtained from ATCC. CHO cells deficient for glycosaminoglycans were derived as previously described (34).

### Inhibition of Glycolysis, Transcription, Translation, and ER-Golgi Transport, or Trypsin or Heparinases I-III Treatment.

Inhibition of glycolysis was performed using the 2-deoxyglucose (2-DG, 5 mM; Sigma) for 48 h. Transcription and translation were inhibited using, respectively, Actinomycin D (5 ug/ml; ICN) or Cycloheximide (1 ug/ml; Millipore) for 18 h. ER-Golgi transport was blocked using Brefeldin A (1:1000) or Monensin (1:1400) (BD Bioscience) for 18 h. Cell surface protein digestion was performed using trypsin (Invitrogen) (1×; 5-10 minutes, 37° C.). Glycosaminoglycans were removed from cells by treatment with heparinases I-III (2 μU/ml) for 30 min in RPMI with no serum. The reaction was then stopped by the addition of PBS-BSA.

### Flow Cytometry

Cells were stained with either sTCR-γδ-PE (10 μg/ml) or negative controls that included Streptavidin-PE (10 ug/ml), IgG-PE (10 μg/ml) (BioLegend), or a sTCRαβ-PE (a kind gift of Dr. Mark Davis). Additional surface staining of T cells consisted of CD4, CD8, CD19, and CD25 (BioLegend). Live-Dead staining (BD Bioscience) was used to eliminate dead cells from analysis. Samples were run on an LSRII flow cytometer (Becton-Dickinson).

### Bioinformatics Analysis

Expression profiling (35) based on Illumina RNA sequencing technology (36) was used to characterize the transcriptomes of 22 of the 24 tumor cell lines examined (excluding bronchoepithelial cell line and 2fTGH). Expression data for all known genes (37) was generated, and those genes whose representation in tetramer-positive cell lines was significantly higher than in negative cell lines were considered as candidate ligands.

### Mass Spectrometry Analysis

Biotinylated sTCR-γδ was bound to avidin-magnetic beads and then incubated with cell lysates from monocytes activated with B. burgdorferi sonicate. Magnetic beads alone served as a negative control. After 4 h, beads were washed 5 times and bound proteins were then separated on polyacrylamide gels. Gel lanes for each sample type were cut into 12 identical regions and diced into 1 mm cubes. In-gel tryptic digestion was conducted on each region as previously described (38). Extracted peptides were subjected to liquid chromatography tandem mass spectrometry (38) except that the analysis was performed using a LTQ linear ion trap mass spectrometer (Thermo Fisher Scientific, Waltham, Mass.). Tandem mass spectra were searched against the forward and reverse concatenated human IPI database using SEQUEST, requiring fully tryptic peptides, allowing a mass tolerance of 2 Da and mass additions of 16 Da for the oxidation of methionine and 71 Da for the addition of acrylamide to cysteine. SEQUEST matches in the first position were then filtered by XCorr scores of 1.8, 2, and 2.7 for singly, doubly, and triply charged ions, respectively. Protein matches made with more than two unique peptides were further considered. This list had a peptide false discovery rate of less than 0.01%.

### Statistical Analysis

The following statistical tests were used: unpaired Student's t-test when comparing two conditions, and a one-way ANOVA with Sidak test for correction for multiple comparisons when comparing multiple variables across multiple conditions.

### Results

Production of a Human Synovial Soluble TCR-γδ(sTCR-γδ) A panel of synovial Vδ1 γδ T cells was previously produced from Lyme arthritis patients (9, 31). A representative clone, Bb15 (Vδ1Vγ9), was selected from which to clone its TCR-γδ. The pBACp10 pH vector has been used previously to produce murine sTCR-γδ tetramers (33). It contains two back-to-back promoters, p10 and polyhedrin, in which the p10 promoter is followed by multiple cloning sites for inserting the γ-chain, and the polyhedrin promoter is followed by multiple cloning sites for inserting the 6-chain (FIG. 1A). Downstream of the γ-chain a hexa-His tag was placed for purification followed by a biotinylation BRP sequence for tetramerization with streptavidin-PE. Protein production was undertaken in Hi5 cells followed by purification using a nickel NTA affinity column. Fractions were analyzed by SDS-PAGE, and those with protein of the correct size were pooled, with yields typically of 1-2 mg per liter of culture. A sample sTCR-γδ preparation is shown in FIG. 1B, stained with Coomassie Blue, showing bands of the expected size for the heterodimer under non-reducing (59 kD) and reducing conditions (30/28 kD for the γ- and δ-chains, respectively). The protein stained by immunoblot with antibodies to either Vδ1 or Cγ (FIG. 1C), and also blocked anti-γδ antibody staining of the synovial γδ T cell clones (FIG. 1D). The purified sTCR-γδ was then biotinylated and tetramerized with streptavidin-PE for use by flow cytometry. As an additional measure of specificity, sTCR-γδ tetramer staining of a fibrosarcoma tumor cell line (2fTGH) could be inhibited by anti-γδ antibody but not control IgG (FIG. 1E). Finally, staining of 2fTGH cells with the sTCR-γδ tetramer was dose-dependent, but did not increase with increasing dose on a negative tumor line, Daudi (FIG. 1F).
Expression of sTCR-γδ Candidate Ligand(s) Varies Among Cell Lines

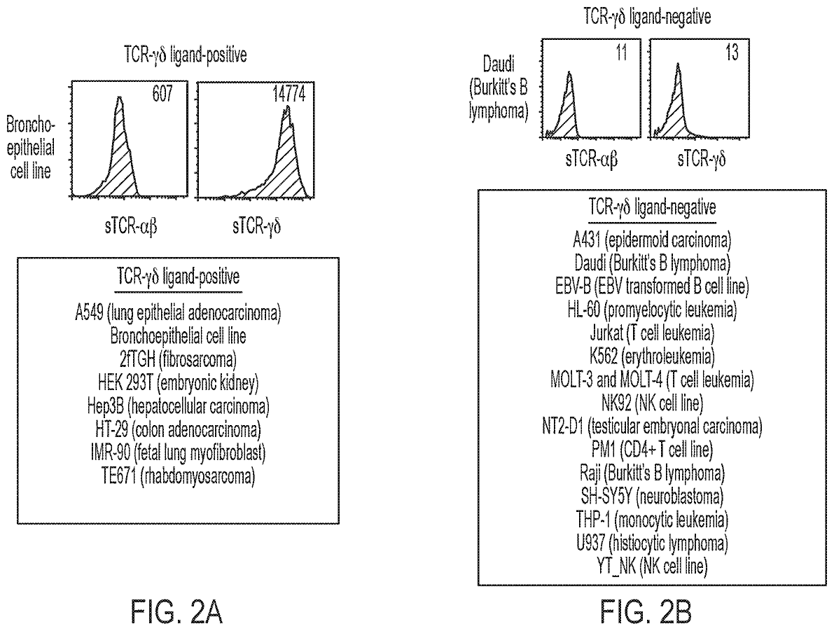

The sTCR-γδ tetramer was initially used to screen a panel of 24 cell lines from a variety of cell sources. None of the cell lines stained with the negative controls (IgG-PE, avidin-PE, or sTCR-αβ tetramer-PE), but the sTCR-γδ tetramer gave a spectrum of staining in which eight cell lines were strongly positive and the other cell lines manifested low to undetectable surface staining (FIG. 2). Of interest was that the positive group was enriched for cell lines of epithelial and fibroblast origin, cell types known to exist where γδ T cells are often found, such as skin, intestines, and synovium. With this information, expression profiling using available RNAseq was used to characterize the transcriptomes of 22 of the 24 tumor cell lines (RNAseq on the bronchoepithelial and 2fTGH were not available). Expression data for all known genes (37) was generated, and those genes whose representation in tetramer-positive cell lines was significantly higher than in negative cell lines were considered to be candidate ligands. This produced an initial list of candidate ligands for sTCR-γδ (shown in Table 2).

Candidate sTCR-γδ Ligands are Sensitive to Trypsin, and Reduced by Inhibition of Transcription, Translation, ER-Golgi Transport, or Removal of Glycosaminoglycans

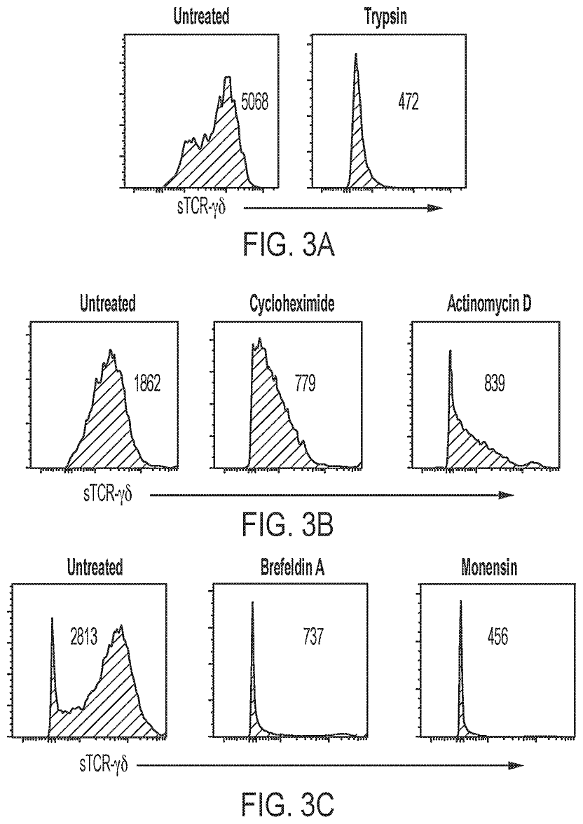

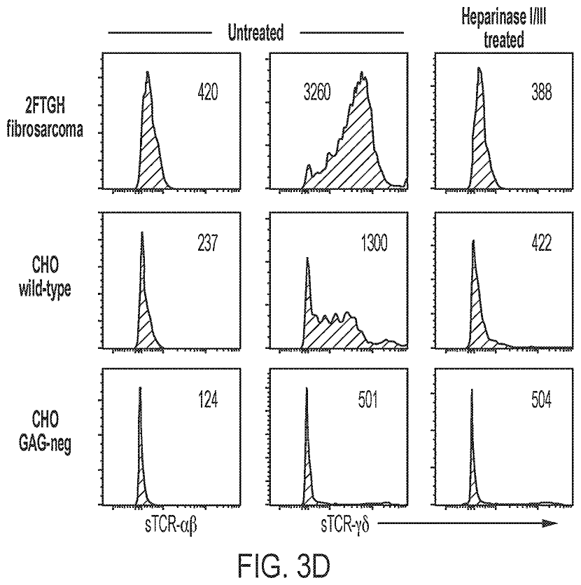

The positively staining cell lines were treated with trypsin and a complete disappearance of surface staining was noted, as exemplified for bronchoepithelial cells in FIG. 3A. Similar results were observed with two additional tumor lines. This supports the view that the TCR-γδ ligand(s) contains a protein component essential for recognition by the receptor. No increase was observed in sTCR-γδ tetramer staining of cells (C1R or HeLa) expressing CD1a, b, c, or d, nor with MICA/B (not shown). Thus, at present there is no evidence that the synovial Vδ1 TCR-γδ ligand(s) is one of these MHC class I-like molecule, at least bound to endogenous molecules from these particular cell lines.

It was further determined that surface TCR-γδ ligand expression was reduced by inhibition of protein translation or transcription with, respectively, cycloheximide or actinomycin D (FIG. 3B). Surface ligand was also considerably reduced by inhibition of transport from the ER to Golgi using either Brefeldin A or Monensin (FIG. 3C). This further demonstrated the protein nature of candidate TCR-γδ ligands. Finally, the extent to which glycosaminoglycans (GAGs) contribute to ligand binding by TCR-γδ was examined. This was tested in two ways. Initially, the ligand-positive fibrosarcoma cell line 2fTGH was either treated or not with heparinases I-III, which removes most GAGs. This considerably reduced sTCR-γδ tetramer staining (FIG. 3D). This was further supported by the observation that sTCR-γδ stained wild-type but not GAG-deficient CHO cells (FIG. 3D).

sTCR-γδ Ligands are Expressed by Activated Monocytes

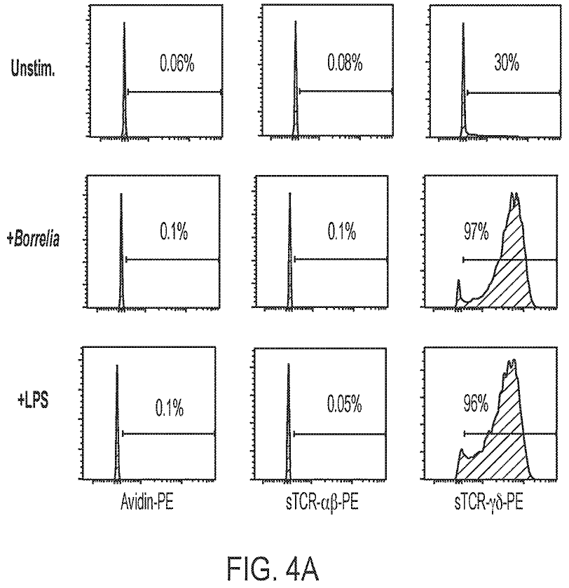

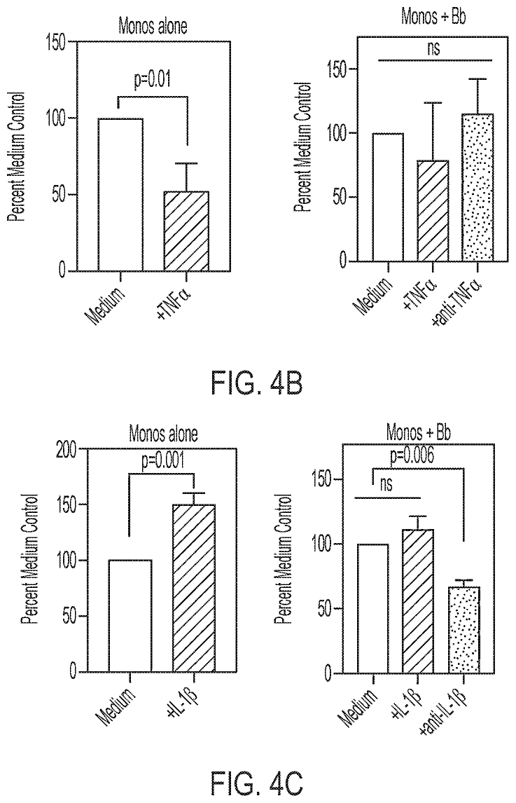

In considering what primary cells might express ligand(s) for the sTCR-γδ, fresh monocytes were first examined, as it had previously been observed that following their activation with Borrelia burgdorferi or LPS, monocytes could activate the synovial γδ T cell clones (31). Consistent with these earlier findings, it was observed that the sTCR-γδ-tetramer did not stain freshly isolated human monocytes, but following 24 hours activation with a sonicate of B. burgdorferi or LPS there was a robust upregulation of sTCR-γδ tetramer staining (FIG. 4). The same cells did not stain with negative controls that included avidin-PE, IgG-PE, or a human sTCR-αβ tetramer-PE. Since activated monocytes are known to produce certain cytokines, particularly TNFα and IL-1β, the possible influence of these cytokines on ligand expression was examined. Surprisingly, the low level of sTCR-γδ tetramer staining of fresh monocytes was reduced yet further with TNFα, whereas ligand expression by Borrelia-activated monocytes was not affected by the further addition of TNFα or blocking anti-TNFα antibody (FIG. 4B). By contrast, IL-1β increased ligand expression by fresh but not activated monocytes, and blocking anti-IL-1β antibody partially inhibited ligand expression by activated monocytes (FIG. 4C). Thus, sTCR-γδ ligand expression appears to be partly regulated by certain monocyte-derived cytokines.

Given the induction of sTCR-γδ ligand expression by activated monocytes, lysates from Borrelia-activated monocytes were prepared. The biotinylated sTCR-γδ complexed with avidin-magnetic beads was then used as a bait. Following incubation with the monocyte lysates, the sTCR-γδ was isolated by magnetic purification, washed five times, and bound proteins were separated on polyacrylamide gels. Gel slices were subjected to trypsin digestion and analyzed by mass spectrometry. Avidin-magnetic beads alone incubated with monocyte lysates served as a negative control. This analysis yielded 291 unique proteins (shown in Table 3). When compared to the list produced by the RNAseq bioinformatics approach of the tumor lines, 16 proteins were found in common. Fourteen unique protein TCR-γδ ligands identified as described herein are listed in Table 1.

TABLE 1
Novel TCR-γδ Ligands - Identified as Common to both
RNAseq Bioinformatics and Mass Spectrometry Analysis

| | EntrezGene | HGNC | |
|---|---|---|---|
| Ensembl Gene ID | ID | symbol | Protein |
| ENSG00000134575 | 53 | ACP2 | acid phosphatase 2 |
| ENSG00000182718 | 302 | ANXA2 | annexin A2 |
| ENSG00000168374 | 378 | ARF4 | ADP ribosylation factor 4 |
| ENSG00000198668 | 805 | CALM1 | calmodulin 1 |
| ENSG00000160014 | 805 | CALM3 | calmodulin 3 |
| ENSG00000127022 | 821 | CANX | calnexin |
| ENSG00000134371 | 79577 | CDC73 | cell division cycle 73 |
| ENSG00000148180 | 2934 | GSN | gelsolin |
| ENSG00000166598 | 7184 | HSP90B1 | heat shock protein 90 beta family |
| | | | member |
| ENSG00000204388 | 3303 | HSPA1B | heat shock protein family A (HSP 70) |
| | | | member 1B |
| ENSG00000133816 | 9645 | MICAL2 | microtubule associated |
| | | | monooxygenase |
| ENSG00000166794 | 5479 | PPIB | peptidylprolyl isomerase B |
| ENSG00000135048 | 23670 | TMEM2 | transmembrane protein 2 |
| ENSG00000140416 | 7168 | TPM1 | tropomyosin 1 |
| ENSG00000035403 | 7414 | VCL | vinculin |
| ENSG00000164924 | 7534 | YWHAZ | tyrosine 3-monooxygenase |

sTCR-γδ Ligands are Expressed by Activated T Cells

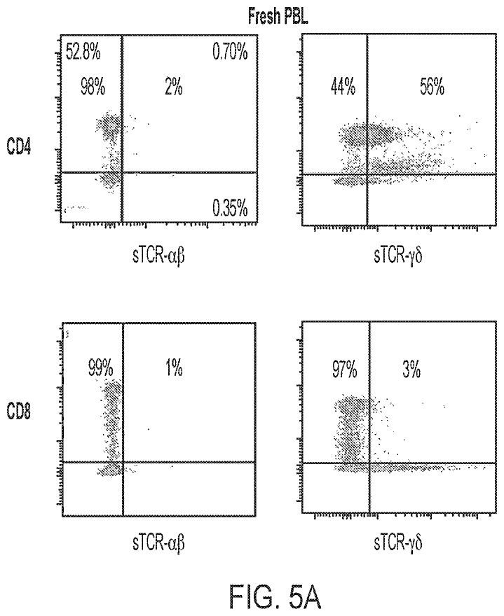

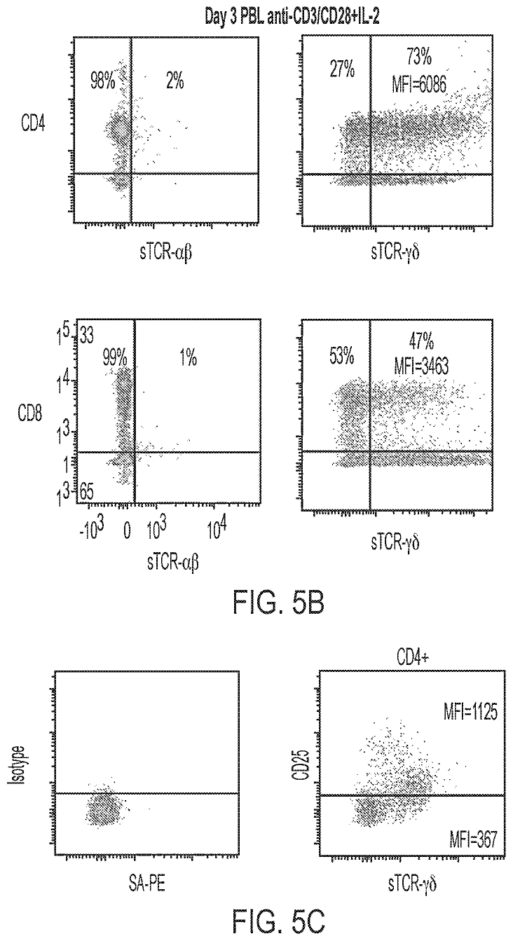

Freshly isolated PBL from three individuals of various ages were further analyzed (28-66). This consistently revealed that fresh CD8⁺ T cells exhibited negligible sTCR-γδ staining, whereas a subset of fresh CD4⁺ T cells manifested modest levels of sTCR-γδ staining (FIG. 5A). In contrast to the freshly isolated T cells, following three days activation with anti-CD3/CD28+IL-2, it was observed that a subset of both CD4⁺ and CD8⁺ T cells now displayed high levels of sTCR-γδ staining (FIG. 5B). Both the proportion of cells expressing ligand and the density was higher on activated CD4⁺ T cells compared to CD8⁺ T cells. Given that in vitro-activated proliferating T cells express sTCR-γδ ligand, it was considered that the subset of fresh CD4⁺ T cells expressing ligand might also represent a proliferative subset. One of the most rapidly proliferative T cell subsets in vivo is Treg cells (35). Treg can be identified as a subset of fresh CD4⁺ T cells expressing CD25. Indeed, when fresh human CD4⁺ T cells based on CD25 expression were subset, sTCR-γδ tetramer staining was again observed preferentially by the CD25⁺ subset (FIG. 5C).

### TCR-γδ Ligand(s) Expression is Partly Dependent Upon Glycolysis

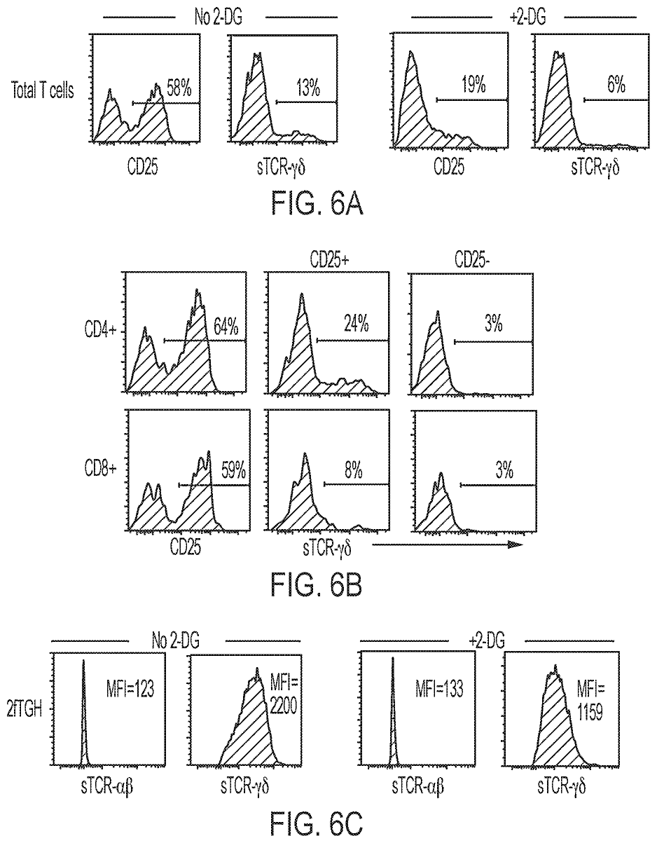

The finding that fresh monocytes and T lymphocytes expressed low to negligible levels of sTCR-γδ ligand(s), but upregulated expression following activation, raised the possibility that this might reflect the known induction of glycolysis following activation of T cells, monocytes, or dendritic cells (36, 37), and the resultant synthetic capacity promoted by glycolysis (38). This notion is supported by the fact that ligand-expressing Treg are also highly glycolytic (35). This question was thus examined in two ways. First, activated T cells were exposed to 2-deoxyglucose (2-DG), an inhibitor of glycolysis. This reduced expression of both CD25 and sTCR-γδ ligand (FIG. 6A). Second, activated T cells on day 3 were distinguished between based on their expression of CD25, as this identifies cells responsive to IL-2 and are hence most glycolytic (38). As shown in FIG. 6B, CD25⁺ T cells expressed sTCR-γδ ligand whereas the CD25-subset was devoid of ligand expression. Of further note is that within the CD25⁺ subset, CD4⁺ T cells again expressed more ligand than CD8⁺ T cells (FIG. 6B). This analysis was extended to the ligand-positive tumor 2fTGH and observed that 2-DG also resulted in reduced ligand expression in these cells (FIG. 6C).

### DISCUSSION

The current findings provide the first unbiased characterization of the spectrum of ligand expression for human synovial Vδ1 γδ T cells. The range of ligand expression may reflect the various locations and seemingly diverse functions attributed to γδ T cells. For example, ligand induction by B. burgdorferi- or LPS-activated monocytes parallels their known ability to activate synovial γδ T cell clones (9, 31). In addition, ligand expression by fresh CD4⁺ but not CD8⁺ T cells also correlates with previous observations that Lyme arthritis synovial γδ T cells suppress by cytolysis the expansion of synovial CD4⁺ but not CD8⁺ T cells in response to B. burgdorferi (9). Finally, defining the spectrum of tumor cell types that express TCR-Vδ1 ligands may help explain which tumors contain Vδ1 γδ T cells, and impact their effectiveness as immunotherapy. The collective findings are also most consistent with the view that γδ T cells respond to self-proteins as much or possibly more than foreign proteins. Although these results were obtained using a sTCR-γδ tetramer from a single synovial γδ T cell clone, the fact that it shares a common Vδ1 chain found on most synovial γδ T cells (9), as well as γδ T cells found in intestinal epithelium (1, 10, 21), several tumors (18), and cells expanded in PBL following certain infections such as HIV (39, 40) and CMV (29), suggests the possibility that Vδ1 γδ T cells from these other sources may share a common physiology of ligand expression.

Previous studies of ligands for murine and human γδ T cells have come largely from the identification of individual molecules that activate a specific γδ T cell clone (27-30). Whereas this has been successful in some instances, the current study applied a broader approach of using a soluble TCR-γδ tetramer in an unbiased fashion to identify the spectrum of ligand expression and how they are regulated. This approach also provided two independent methods by which to identify candidate ligands. One method used RNAseq transcriptome analysis from 22 tumor cell lines to match genes increased in positively staining tumors and decreased in negatively staining tumors. The second approach used the sTCR-γδ tetramer as a bait to bind ligands from lysates of activated monocytes, and then identified the bound proteins by mass spectrometry. It is of considerable interest that among these two sets of candidate ligands were 16 in common, two of which, Annexin A2 and heat shock protein 70, have been previously proposed as ligands for γδ T cells (39-41). On the other hand, surface sTCR-γδ tetramer binding was eliminated by treatment with trypsin or removal of GAGs, and also suppressed by inhibition of ER-Golgi transport, suggesting the involvement of a combination of protein and GAGs in tetramer binding.

Although the findings thus far have not determined whether this represents one or several TCR-γδ ligands, they do provide a framework for understanding the distribution and regulation of ligand expression, which is critical for better understanding of γδ T cell biology. For example, γδ T cells have been implicated in the defense against a variety of infections (2-7), which is consistent with the finding that various TLR agonists induce TCR-γδ ligand expression on monocytes. Similar studies using a murine soluble TCR-76 also found ligands induced with bacterial infection (21). In addition, γδ T cells have been found to generally ameliorate various autoimmune models (12-15), which may be consistent with the expression of ligand by a subset of activated CD4⁺ T cells.

The induction of TCR-γδ ligand expression by activation of primary monocytes or T cells, as well as ligand expression by a variety of highly proliferative tumor cell lines, suggested that the metabolic state of cells may influence their ability to express TCR-γδ ligands. Activation of monocytes and T cells is known to induce a metabolic switch to glycolysis to provide the synthetic capacity for proliferation (36, 37). In addition, Treg, which are known to be glycolytic in vivo (35), spontaneously expressed ligand. Moreover, most tumors are highly glycolytic, and the inhibition of glycolysis in these cells also reduced ligand expression. Collectively, these findings suggest that some γδ T cells may function to survey and regulate highly proliferative cells.

It is of some interest that the cell lines bearing high levels of TCR-γδ ligand expression were enriched for those of epithelial and fibroblast origin, since Vδ1 γδ T cells are typically found at epithelial barriers, such as skin, intestinal epithelium, and in inflamed synovium, which is rich in fibroblasts (41). By contrast, sTCR-γδ ligand expression was noticeably absent from most tumor lines of hematopoietic origin. The spectrum of tumor staining with the human synovial sTCR-γδ also bears considerable similarity to results using a murine sTCR-γδ, which strongly stained epithelial and fibroblast tumors, and less well tumors of hematopoietic origin (33). These same murine sTCR-γδ also stained macrophages activated by TLR2 or TLR4 stimuli, similar to the findings with monocytes activated by Borrelia or LPS (42). Furthermore, staining of macrophages by the murine sTCR-γδ was also not affected by the absence of P2-microgloublin, suggesting little or no contribution of ligand by classical or non-classical MHC class I molecules. This agrees with the findings that the human synovial sTCR-γδ tetramer staining was not affected by the presence or absence of CD1 or MICA/B molecules.

The expression of ligand(s) by transformed cell lines suggests routes to identification of the TCR-γδ ligand for synovial Vδ1 T cells. The variation in ligand expression by the various tumor cell lines from negligible to high lends itself to an RNA-seq and bioinformatics approach to match expression levels of genes with the ligand expression as detected by the sTCR-γδ tetramer. This may provide a powerful tool by which to identify candidate TCR-γδ ligands in an unbiased fashion. This could be followed by CRISPR/Cas9 deletion of candidates to identify the ligand(s) as well as their regulatory pathways of synthesis and transport (43).

The findings in this study were made using primary cells or tumor cell lines. Future studies will attempt to extend these results to analyses of sTCR-γδ tetramer histologic staining of primary tissues as well as tumors and inflamed synovium to determine the spectrum of TCR-γδ ligand expression at these sites. Screening primary tumors for binding of TCR-γδ tetramer may also help identify tumors that may benefit from immunotherapy with Vδ1 γδ T cells. In addition, identifying the ligands in inflamed synovium or intestinal epithelium will provide therapeutic strategies for manipulating the function of infiltrating γδ T cells.

### REFERENCES

1. Born, W., C. Cady, J. Jones-Carson, A. Mukasa, M. Lahn, and R. O'Brien. 1999. Immunoregulatory functions of gamma delta T cells. Advances in Immunology 71: 77-144.
2. Shi, C., B. Sahay, J. Q. Russell, K. A. Fortner, N. Hardin, T. J. Sellati, and R. C. Budd. 2011. Reduced immune response to Borrelia burgdorferi in the absence of γδ T cells. Infect. Immun. 79: 3940-3946.
3. Hiromatsu, K., Y. Yoshikai, G. Matsuzaki, S. Ohga, K. Muramori, K. Matsumoto, J. A. Bluestone, and K. Nomoto. 1992. A protective role of gamma/delta T cells in primary infection with Listeria monocytogenes in mice. J Exp Med 175: 49-56.
4. Rosat, J. P., H. R. MacDonald, and J. A. Louis. 1993. A role for gamma delta+T cells during experimental infection of mice with Leishmania major. J Immunol 150: 550-555.
5. Kaufmann, S. H., and C. H. Ladel. 1994. Role of T cell subsets in immunity against intracellular bacteria: experimental infections of knock-out mice with Listeria monocytogenes and Mycobacterium bovis BCG. Immunobiology 191: 509-519.
6. Tsuji, M., P. Mombaerts, L. Lefrancois, R. S. Nussenzweig, F. Zavala, and S. Tonegawa. 1994. Gamma delta T cells contribute to immunity against the liver stages of malaria in alpha beta T-cell-deficient mice. Proc Nat Acad Sci USA 91: 345-349.
7. Mixter, P. F., V. Camerini, B. J. Stone, V. L. Miller, and M. Kronenberg. 1994. Mouse T lymphocytes that express a gamma delta T-cell antigen receptor contribute to resistance to Salmonella infection in vivo. Infect Immun 62: 4618-4621.
8. Brennan, F. M., M. Londei, A. M. Jackson, T. Hercend, M. B. Brenner, R. N. Maini, and M. Feldmann. 1988. T cells expressing gamma delta chain receptors in rheumatoid arthritis. J Autoimmun 1: 319-326.
9. Vincent, M., K. Roessner, D. Lynch, S. M. Cooper, L. H. Sigal, and R. C. Budd. 1996. Apoptosis of Fas high CD4+ Synovial T cells by Borrelia reactive Fas Ligand high gamma delta T cells in Lyme Arthritis. J. Exp. Med. 184: 2109-2117.
10. Rust, C., Y. Kooy, S. Pena, M. L. Mearin, P. Kluin, and F. Koning. 1992. Phenotypical and functional characterization of small intestinal TcR gamma delta+T cells in coeliac disease. Scand J Immunol 35: 459-468.
11. Balbi, B., D. R. Moller, M. Kirby, K. J. Holroyd, and R. G. Crystal. 1990. Increased numbers of T lymphocytes with gamma delta-positive antigen receptors in a subgroup of individuals with pulmonary sarcoidosis. J Clin Invest 85: 1353-1361.
12. Peterman, G. M., C. Spencer, A. I. Sperling, and J. A. Bluestone. 1993. Role of gamma delta T cells in murine collagen-induced arthritis. J Immunol 151: 6546-6558.
13. Pelegri, C., P. Kuhnlein, E. Buchner, C. B. Schmidt, A. Franch, M. Castell, T. Hunig, F. Emmrich, and R. W. Kinne. 1996. Depletion of gamma/delta T cells does not prevent or ameliorate, but rather aggravates, rat adjuvant arthritis. Arthritis Rheum 39: 204-215.
14. Peng, S. L., M. P. Madaio, A. C. Hayday, and J. Craft. 1996. Propagation and regulation of systemic autoimmunity by gamma delta T cells. J Immunol 157: 5689-5698.
15. Mukasa, A., K. Hiromatsu, G. Matsuzaki, R. O'Brien, W. Born, and K. Nomoto. 1995. Bacterial infection of the testis leading to autoaggressive immunity triggers apparently opposed responses of alpha beta and gamma delta T cells. J Immunol 155: 2047-2056.
16. Girardi, M., D. E. Oppenheim, C. R. Steele, J. M. Lewis, E. Glusac, R. Filler, P. Hobby, B. Sutton, R. E. Tigelaar, and A. C. Hayday. 2001. Regulation of cutaneous malignancy by gammadelta T cells. Science 294: 605-609.
17. Costa, G., S. Loizon, M. Guenot, I. Mocan, F. Halary, G. de Saint-Basile, V. Pitard, J. Dechanet-Merville, J. F. Moreau, M. Troye-Blomberg, O. Mercereau-Puijalon, and C. Behr. 2011. Control of Plasmodium falciparum erythrocytic cycle: gammadelta T cells target the red blood cell-invasive merozoites. Blood 118: 6952-6962.
18. Gentles, A. J., A. M. Newman, C. L. Liu, S. V. Bratman, W. Feng, D. Kim, V. S. Nair, Y. Xu, A. Khuong, C. D. Hoang, M. Diehn, R. B. West, S. K. Plevritis, and A. A. Alizadeh. 2015. The prognostic landscape of genes and infiltrating immune cells across human cancers. Nat Med 21: 938-945.
19. Wilhelm, M., V. Kunzmann, S. Eckstein, P. Reimer, F. Weissinger, T. Ruediger, and H. P. Tony. 2003. Gammadelta T cells for immune therapy of patients with lymphoid malignancies. Blood 102: 200-206.
20. Zeng, X., Wei, Y-L., Huang, J., Newell, E. W., Yu, H., Kidd, B. A., Kuhns, M. S., Waters, R. W., Davis, M. M., Weaver, C. T., and Chien, Y-h. 2012. Gamma delta T cells recognize a microbial encoded B cell antigen to initiate a rapid antigen-specific interleukin-17 response. Immunity 37: 524-534.
21. Nielsen, M. M., D. A. Witherden, and W. L. Havran. 2017. gammadelta T cells in homeostasis and host defence of epithelial barrier tissues. Nat Rev Immunol 17: 733-745.
22. Hirsh, M. I., and W. G. Junger. 2008. Roles of heat shock proteins and gamma delta T cells in inflammation. Am J Respir Cell Mol Biol 39: 509-513.
23. Morita, C. T., E. M. Beckman, J. F. Bukowski, Y. Tanaka, H. Band, B. R. Bloom, D. E. Golan, and M. B. Brenner. 1995. Direct presentation of nonpeptide prenyl pyrophosphate antigens to human gamma delta T cells. Immunity 3: 495-507.
24. Tanaka, Y., C. T. Morita, E. Nieves, M. B. Brenner, and B. R. Bloom. 1995. Natural and synthetic non-peptide antigens recognized by human gamma delta T cells. Nature 375: 155-158.
25. Bukowski, J. F., C. T. Morita, and M. B. Brenner. 1999. Human gamma delta T cells recognize alkylamines derived from microbes, edible plants, and tea: implications for innate immunity. Immunity 11: 57-65.
26. Witherden, D. A., K. Ramirez, and W. L. Havran. 2014. Multiple Receptor-Ligand Interactions Direct Tissue-Resident gammadelta T Cell Activation. Front Immunol 5: 602.
27. Adams, E. J., Y. H. Chien, and K. C. Garcia. 2005. Structure of a gammadelta T cell receptor in complex with the nonclassical MHC T22. Science 308: 227-231.
28. Chien, Y. H., and Y. Konigshofer. 2007. Antigen recognition by gammadelta T cells. Immunological Reviews 215: 46-58.
29. Willcox, C. R., Pitard, V., Netzer, S., Couzi, L., Salim, M., Silberzahn, T., Moreau, J-F., Hayday, A. C., Willcox, B. E., Dechanet-Merville, J. 2012. Cytomegalovirus and tumor stress surveillance by binding of a human gamma delta T cell antigen receptor to endothelial protein C receptor. Nat. Immunol. 13: 872-880.
30. Luoma, A. M., C. D. Castro, T. Mayassi, L. A. Bembinster, L. Bai, D. Picard, B. Anderson, L. Scharf, J. E. Kung, L. V. Sibener, P. B. Savage, B. Jabri, A. Bendelac, and E. J. Adams. 2013. Crystal structure of V1 T cell receptor in complex with CD1d-sulfatide shows MHC-like recognition of a self-lipid by human T cells. Immunity 39: 1032-1042.
31. Vincent, M. S., K. Roessner, T. Sellati, C. D. Huston, L. H. Sigal, S. M. Behar, J. D. Radolf, and R. C. Budd. 1998. Lyme arthritis synovial gamma delta T cells respond to Borrelia burgdorferi lipoproteins and lipidated hexapeptides. J Immunol 161: 5762-5771.
32. Kappler, J., J. White, H. Kozono, J. Clements, and P. Marrack. 1994. Binding of a soluble alpha beta T-cell receptor to superantigen/major histocompatibility complex ligands. Proc Natl Acad Sci USA 91: 8462-8466.
33. Aydintug, M. K., C. L. Roark, X. Yin, J. M. Wands, W. K. Born, and R. L. O'Brien. 2004. Detection of cell surface ligands for the gamma delta TCR using soluble TCRs. J Immunol 172: 4167-4175.
34. Esko, J. D., T. E. Stewart, and W. H. Taylor. 1985. Animal cell mutants defective in glycosaminoglycan biosynthesis. Proc Natl Acad Sci USA 82: 3197-3201.
35. 't Hoen, P. A., Y. Ariyurek, H. H. Thygesen, E. Vreugdenhil, R. H. Vossen, R. X. de Menezes, J. M. Boer, G. J. van Ommen, and J. T. den Dunnen. 2008. Deep sequencing-based expression analysis shows major advances in robustness, resolution and inter-lab portability over five microarray platforms. Nucleic Acids Res 36: e141.
36. Bentley et al., 2008. Accurate whole human genome sequencing using reversible terminator chemistry. Nature 456: 53-59.
37. Hubbard et al., 2009. Ensembl 2009. Nucleic Acids Res 37: D690-697.
38. Ballif, B. A., Z. Cao, D. Schwartz, K. L. Carraway, 3rd, and S. P. Gygi. 2006. Identification of 14-3-3epsilon substrates from embryonic murine brain. J Proteome Res 5: 2372-2379
39. Marlin, R., A. Pappalardo, H. Kaminski, C. R. Willcox, V. Pitard, S. Netzer, C. Khairallah, A. M. Lomenech, C. Harly, M. Bonneville, J. F. Moreau, E. Scotet, B. E. Willcox, B. Faustin, and J. Dechanet-Merville. 2017. Sensing of cell stress by human gammadelta TCR-dependent recognition of annexin A2. Proc Natl Acad Sci USA 114: 3163-3168.
40. Born, W., L. Hall, A. Dallas, J. Boymel, T. Shinnick, D. Young, P. Brennan, and R. O'Brien. 1990. Recognition of a peptide antigen by heat shock-reactive gamma delta T lymphocytes. Science 249: 67-69.
41. Chen, H., X. He, Z. Wang, D. Wu, H. Zhang, C. Xu, H. He, L. Cui, D. Ba, and W. He. 2008. Identification of human T cell receptor gammadelta-recognized epitopes/proteins via CDR3delta peptide-based immunobiochemical strategy. Journal of Biological Chemistry 283: 12528-12537.
42. Galgani, M., V. De Rosa, A. La Cava, and G. Matarese. 2016. Role of Metabolism in the Immunobiology of Regulatory T Cells. J Immunol 197: 2567-2575.
43. Kaminski, M. M., S. W. Sauer, M. Kaminski, S. Opp, T. Ruppert, P. Grigaravicius, P. Grudnik, H. J. Grone, P. H. Krammer, and K. Gulow. 2012. T cell activation is driven by an ADP-dependent glucokinase linking enhanced glycolysis with mitochondrial reactive oxygen species generation. Cell Rep 2: 1300-1315.
44. Everts, B., E. Amiel, S. C. Huang, A. M. Smith, C. H. Chang, W. Y. Lam, V. Redmann, T. C. Freitas, J. Blagih, G. J. van der Windt, M. N. Artyomov, R. G. Jones, E. L. Pearce, and E. J. Pearce. 2014. TLR-driven early glycolytic reprogramming via the kinases TBK1-IKKvarepsilon supports the anabolic demands of dendritic cell activation. Nat Immunol 15: 323-332.
45. Vander Heiden, M. G., L. C. Cantley, and C. B. Thompson. 2009. Understanding the Warburg effect: the metabolic requirements of cell proliferation. Science 324: 1029-1033.
46. Li, Z., Y. Jiao, Y. Hu, L. Cui, D. Chen, H. Wu, J. Zhang, and W. He. 2015. Distortion of memory Vdelta2 gammadelta T cells contributes to immune dysfunction in chronic HIV infection. Cell Mol Immunol 12: 604-614.
47. Li, Z., W. Li, N. Li, Y. Jiao, D. Chen, L. Cui, Y. Hu, H. Wu, and W. He. 2014. gammadelta T cells are involved in acute HIV infection and associated with AIDS progression. PLoS One 9: e106064.
48. Firestein, G. S. 2005. Immunologic mechanisms in the pathogenesis of rheumatoid arthritis. J Clin Rheumatol 11: S39-44.
49. Aydintug, M. K., C. L. Roark, J. L. Chain, W. K. Born, and R. L. O'Brien. 2008. Macrophages express multiple ligands for gammadelta TCRs. Mol Immunol 45: 3253-3263.

TABLE 2

| | | UniProt | Gene | |
|---|---|---|---|---|
| Adj P Value | Ensembl ID | Accession | Symbol | Protein Description |
| 8.727E−06 | ENSG00000135048 | Q9UHN6 | TMEM2 | transmembrane protein 2 |
| 1.12469E−05 | ENSG00000099377 | Q9H2F3 | HSD3B7 | hydroxy-delta-5-steroid |
| | | | | dehydrogenase, 3 beta- and |
| | | | | steroid delta-isomerase 7 |
| 4.24595E−05 | ENSG00000070961 | P20020 | ATP2B1 | ATPase plasma membrane Ca2+ |
| | | | | transporting 1 |
| 4.24595E−05 | ENSG00000177707 | Q9NQS3 | NECTIN3 | nectin cell adhesion molecule 3 |
| 6.17456E−05 | ENSG00000162627 | Q9UNH6 | SNX7 | sorting nexin 7 |
| 7.36045E−05 | ENSG00000150938 | Q9NZV1 | CRIM1 | cysteine rich transmembrane |
| | | | | BMP regulator 1 |
| 8.38219E−05 | ENSG00000104067 | Q07157 | TJP1 | tight junction protein 1 |
| 0.000108283 | ENSG00000100934 | Q15436 | SEC23A | Sec23 homolog A, coat complex |
| | | | | II component |
| 0.000108283 | ENSG00000105438 | P24390 | KDELR1 | KDEL endoplasmic reticulum |
| | | | | protein retention receptor 1 |
| 0.000108283 | ENSG00000177700 | P62875 | POLR2L | RNA polymerase II subunit L |
| 0.000116746 | ENSG00000082781 | P18084 | ITGB5 | integrin subunit beta 5 |
| 0.000116746 | ENSG00000105518 | Q6UW68 | TMEM205 | transmembrane protein 205 |
| 0.000132707 | ENSG00000120129 | P28562 | DUSP1 | dual specificity phosphatase 1 |
| 0.000134172 | ENSG00000111897 | Q9NRX5 | SERINC1 | serine incorporator 1 |
| 0.000150726 | ENSG00000136153 | Q8WWI1 | LMO7 | LIM domain 7 |
| 0.000184924 | ENSG00000126524 | Q9Y3A5 | SBDS | SBDS ribosome assembly |
| | | | | guanine nucleotide exchange |
| | | | | factor |
| 0.000205879 | ENSG00000156642 | Q9Y639 | NPTN | neuroplastin |
| 0.000238303 | ENSG00000161011 | Q13501 | SQSTM1 | sequestosome 1 |
| 0.000238546 | ENSG00000109814 | O60701 | UGDH | UDP-glucose 6-dehydrogenase |
| 0.000254753 | ENSG00000121039 | Q8IZV5 | RDH10 | retinol dehydrogenase 10 (all- |
| | | | | trans) |
| 0.000267084 | ENSG00000144426 | Q6ZS30 | NBEAL1 | neurobeachin like 1 |
| 0.000274088 | ENSG00000115561 | Q9Y3E7 | CHMP3 | charged multivesicular body |
| | | | | protein 3 |
| 0.000294047 | ENSG00000184349 | P52803 | EFNA5 | ephrin A5 |
| 0.000298626 | ENSG00000141736 | P04626 | ERBB2 | erb-b2 receptor tyrosine kinase |
| | | | | 2 |
| 0.000343652 | ENSG00000169814 | P43251 | BTD | biotinidase |
| 0.000364181 | ENSG00000105997 | O43365 | HOXA3 | homeobox A3 |
| 0.000379182 | ENSG00000143878 | P62745 | RHOB | ras homolog family member B |
| 0.000408468 | ENSG00000102158 | Q9H0U3 | MAGT1 | magnesium transporter 1 |
| 0.000408468 | ENSG00000127022 | P27824 | CANX | calnexin |
| 0.000408468 | ENSG00000157110 | Q93062 | RBPMS | RNA binding protein with |
| | | | | multiple splicing |
| 0.000408468 | ENSG00000177628 | P04062 | GBA | glucosylceramidase beta |
| 0.000408468 | ENSG00000213949 | P56199 | ITGA1 | integrin subunit alpha 1 |
| 0.000427348 | ENSG00000180488 | Q8NAN2 | MIGA1 | mitoguardin 1 |
| 0.000429719 | ENSG00000067167 | Q15629 | TRAM1 | translocation associated |
| | | | | membrane protein 1 |
| 0.000429719 | ENSG00000113583 | Q8NC54 | C5orf15 | chromosome 5 open reading |
| | | | | frame 15 |
| 0.00043047 | ENSG00000071859 | Q14320 | FAM50A | family with sequence similarity |
| | | | | 50 member A |
| 0.00043047 | ENSG00000117500 | Q9Y3A6 | TMED5 | transmembrane p24 trafficking |
| | | | | protein 5 |
| 0.000438015 | ENSG00000152291 | O43493 | TGOLN2 | trans-golgi network protein 2 |
| 0.000465338 | ENSG00000110435 | O00330 | PDHX | pyruvate dehydrogenase |
| | | | | complex component X |
| 0.000486375 | ENSG00000120885 | P10909 | CLU | clusterin |
| 0.000497883 | ENSG00000153234 | P43354 | NR4A2 | nuclear receptor subfamily 4 |
| | | | | group A member 2 |
| 0.000500057 | ENSG00000097033 | Q9Y371 | SH3GLB1 | SH3 domain containing GRB2 |
| | | | | like endophilin B1 |
| 0.000503234 | ENSG00000189171 | Q99584 | S100A13 | S100 calcium binding protein |
| | | | | A13 |
| 0.000546155 | ENSG00000144959 | Q6PIU2 | NCEH1 | neutral cholesterol ester |
| | | | | hydrolase 1 |
| 0.000547849 | ENSG00000176485 | P53816 | PLA2G16 | phospholipase A2 group XVI |
| 0.000580975 | ENSG00000186854 | Q86V40 | TRABD2A | TraB domain containing 2A |
| 0.000597084 | ENSG00000131871 | Q9BQE4 | VIMP | VCP interacting membrane |
| | | | | selenoprotein |
| 0.000599639 | ENSG00000142279 | A6NIX2 | WTIP | Wilms tumor 1 interacting |
| | | | | protein |
| 0.000601922 | ENSG00000135083 | Q8IV13 | CCNJL | cyclin J like |
| 0.000616836 | ENSG00000173402 | Q14118 | DAG1 | dystroglycan 1 |
| 0.000620152 | ENSG00000105854 | Q15165 | PON2 | paraoxonase 2 |
| 0.000627419 | ENSG00000178307 | P17152 | TMEM11 | transmembrane protein 11 |
| 0.000632399 | ENSG00000132254 | P53365 | ARFIP2 | ADP ribosylation factor |
| | | | | interacting protein 2 |
| 0.000632399 | ENSG00000139289 | Q8WV24 | PHLDA1 | pleckstrin homology like domain |
| | | | | family A member 1 |
| 0.000639594 | ENSG00000168615 | Q13443 | ADAM9 | ADAM metallopeptidase domain |
| | | | | 9 |
| 0.00064241 | ENSG00000168056 | Q9NS15 | LTBP3 | latent transforming growth |
| | | | | factor beta binding protein 3 |
| 0.000661187 | ENSG00000103852 | Q5W5X9 | TTC23 | tetratricopeptide repeat domain |
| | | | | 23 |
| 0.000668266 | ENSG00000064651 | P55011 | SLC12A2 | solute carrier family 12 member |
| | | | | 2 |
| 0.000668266 | ENSG00000134970 | Q9Y3B3 | TMED7 | transmembrane p24 trafficking |
| | | | | protein 7 |
| 0.000668266 | ENSG00000161013 | Q9UQ53 | MGAT4B | mannosyl (alpha-1,3-)- |
| | | | | glycoprotein beta-1,4-N- |
| | | | | acetylglucosaminyltransferase, |
| | | | | isozyme B |
| 0.000670886 | ENSG00000115486 | P38435 | GGCX | gamma-glutamyl carboxylase |
| 0.000693066 | ENSG00000134318 | O75116 | ROCK2 | Rho associated coiled-coil |
| | | | | containing protein kinase 2 |
| 0.000702505 | ENSG00000136240 | P33947 | KDELR2 | KDEL endoplasmic reticulum |
| | | | | protein retention receptor 2 |
| 0.000739742 | ENSG00000130402 | O43707 | ACTN4 | actinin alpha 4 |
| 0.000777099 | ENSG00000129925 | Q9HCN3 | TMEM8A | transmembrane protein 8A |
| 0.000780067 | ENSG00000183978 | Q9Y2R0 | COA3 | cytochrome c oxidase assembly |
| | | | | factor 3 |
| 0.000839921 | ENSG00000168036 | P35222 | CTNNB1 | catenin beta 1 |
| 0.000892828 | ENSG00000196141 | Q9NUQ6 | SPATS2L | spermatogenesis associated |
| | | | | serine rich 2 like |
| 0.000906307 | ENSG00000151327 | Q8N128 | FAM177A1 | family with sequence similarity |
| | | | | 177 member A1 |
| 0.000951691 | ENSG00000129515 | Q9UNH7 | SNX6 | sorting nexin 6 |
| 0.000954051 | ENSG00000074319 | Q99816 | TSG101 | tumor susceptibility 101 |
| 0.000969894 | ENSG00000198925 | Q7Z3C6 | ATG9A | autophagy related 9A |
| 0.000975843 | ENSG00000172830 | Q8TE77 | SSH3 | slingshot protein phosphatase 3 |
| 0.000994688 | ENSG00000129422 | Q9ULD2 | MTUS1 | microtubule associated tumor |
| | | | | suppressor 1 |
| 0.001012224 | ENSG00000130669 | O96013 | PAK4 | p21 (RAC1) activated kinase 4 |
| 0.001019111 | ENSG00000075420 | Q53EP0 | FNDC3B | fibronectin type III domain |
| | | | | containing 3B |
| 0.001073946 | ENSG00000137831 | Q9BZF9 | UACA | uveal autoantigen with coiled- |
| | | | | coil domains and ankyrin |
| | | | | repeats |
| 0.001143087 | ENSG00000153317 | Q9ULH1 | ASAP1 | ArfGAP with SH3 domain, |
| | | | | ankyrin repeat and PH domain 1 |
| 0.001143087 | ENSG00000179134 | Q5PRF9 | SAMD4B | sterile alpha motif domain |
| | | | | containing 4B |
| 0.001162149 | ENSG00000013563 | P49184 | DNASE1L1 | deoxyribonuclease 1 like 1 |
| 0.001173519 | ENSG00000134575 | P11117 | ACP2 | acid phosphatase 2, lysosomal |
| 0.001176938 | ENSG00000112473 | Q92504 | SLC39A7 | solute carrier family 39 member |
| | | | | 7 |
| 0.001181892 | ENSG00000011405 | O00443 | PIK3C2A | phosphatidylinositol-4- |
| | | | | phosphate 3-kinase catalytic |
| | | | | subunit type 2 alpha |
| 0.001181892 | ENSG00000100292 | P09601 | HMOX1 | heme oxygenase 1 |
| 0.00121557 | ENSG00000077147 | Q9HD45 | TM9SF3 | transmembrane 9 superfamily |
| | | | | member 3 |
| 0.001227828 | ENSG00000136603 | P12757 | SKIL | SKI-like proto-oncogene |
| 0.001237235 | ENSG00000140836 | Q15911 | ZFHX3 | zinc finger homeobox 3 |
| 0.001237235 | ENSG00000174437 | P16615 | ATP2A2 | ATPase |
| | | | | sarcoplasmic/endoplasmic |
| | | | | reticulum Ca2+ transporting 2 |
| 0.001293062 | ENSG00000109501 | O76024 | WFS1 | wolframin ER transmembrane |
| | | | | glycoprotein |
| 0.001324663 | ENSG00000123983 | O95573 | ACSL3 | acyl-CoA synthetase long-chain |
| | | | | family member 3 |
| 0.001324663 | ENSG00000160211 | P11413 | G6PD | glucose-6-phosphate |
| | | | | dehydrogenase |
| 0.001353678 | ENSG00000164403 | Q2M3G4 | SHROOM1 | shroom family member 1 |
| 0.001355632 | ENSG00000074696 | Q9P035 | HACD3 | 3-hydroxyacyl-CoA dehydratase |
| | | | | 3 |
| 0.001355632 | ENSG00000177606 | P05412 | JUN | Jun proto-oncogene, AP-1 |
| | | | | transcription factor subunit |
| 0.001364941 | ENSG00000114346 | Q9H8V3 | ECT2 | epithelial cell transforming 2 |
| 0.001388012 | ENSG00000164144 | P53367 | ARFIP1 | ADP ribosylation factor |
| | | | | interacting protein 1 |
| 0.001388236 | ENSG00000101928 | Q9UJG1 | MOSPD1 | motile sperm domain containing |
| | | | | 1 |
| 0.001428426 | ENSG00000134352 | P40189 | IL6ST | interleukin 6 signal transducer |
| 0.001433873 | ENSG00000148248 | O15260 | SURF4 | surfeit 4 |
| 0.001458624 | ENSG00000137203 | P05549 | TFAP2A | transcription factor AP-2 alpha |
| 0.001458624 | ENSG00000144366 | Q9UBP9 | GULP1 | GULP, engulfment adaptor PTB |
| | | | | domain containing 1 |
| 0.001547194 | ENSG00000123575 | Q6PEV8 | FAM199X | family with sequence similarity |
| | | | | 199, X-linked |
| 0.001547194 | ENSG00000181045 | Q86WA9 | SLC26A11 | solute carrier family 26 member |
| | | | | 11 |
| 0.00155724 | ENSG00000136052 | Q96JW4 | SLC41A2 | solute carrier family 41 member |
| | | | | 2 |
| 0.00159729 | ENSG00000197982 | Q6ZSJ8 | C1orf122 | chromosome 1 open reading |
| | | | | frame 122 |
| 0.001623621 | ENSG00000075234 | Q5R3I4 | TTC38 | tetratricopeptide repeat domain |
| | | | | 38 |
| 0.001661172 | ENSG00000085721 | Q9NYV6 | RRN3 | RRN3 homolog, RNA |
| | | | | polymerase 1 transcription |
| | | | | factor |
| 0.001673179 | ENSG00000203950 | Q9BWD3 | FAM127B | family with sequence similarity |
| | | | | 127 member B |
| 0.001739791 | ENSG00000102531 | Q9Y2H6 | FNDC3A | fibronectin type III domain |
| | | | | containing 3A |
| 0.001743115 | ENSG00000173517 | Q9H792 | PEAK1 | pseudopodium enriched |
| | | | | atypical kinase 1 |
| 0.001789096 | ENSG00000042286 | Q9BRQ8 | AIFM2 | apoptosis inducing factor, |
| | | | | mitochondria associated 2 |
| 0.001809917 | ENSG00000113070 | Q99075 | HBEGF | heparin binding EGF like growth |
| | | | | factor |
| 0.001838822 | ENSG00000146476 | Q9H993 | ARMT1 | acidic residue methyltransferase |
| | | | | 1 |
| 0.001846394 | ENSG00000122203 | Q96A73 | KIAA1191 | KIAA1191 |
| 0.001859923 | ENSG00000139211 | Q86SJ2 | AMIGO2 | adhesion molecule with Ig like |
| | | | | domain 2 |
| 0.001887225 | ENSG00000140391 | O60637 | TSPAN3 | tetraspanin 3 |
| 0.001959442 | ENSG00000117298 | P42892 | ECE1 | endothelin converting enzyme 1 |
| 0.001964189 | ENSG00000175582 | P20340 | RAB6A | RAB6A, member RAS oncogene |
| | | | | family |
| 0.001965812 | ENSG00000165410 | Q9Y281 | CFL2 | cofilin 2 |
| 0.001972901 | ENSG00000112245 | Q93096 | PTP4A1 | protein tyrosine phosphatase |
| | | | | type IVA, member 1 |
| 0.002008104 | ENSG00000151239 | Q12792 | TWF1 | twinfilin actin binding protein 1 |
| 0.002035684 | ENSG00000114988 | Q9H0V9 | LMAN2L | lectin, mannose binding 2 like |
| 0.002035684 | ENSG00000146425 | P63172 | DYNLT1 | dynein light chain Tctex-type 1 |
| 0.002073082 | ENSG00000140526 | P08910 | ABHD2 | abhydrolase domain containing |
| | | | | 2 |
| 0.002111466 | ENSG00000075426 | P15408 | FOSL2 | FOS like 2, AP-1 transcription |
| | | | | factor subunit |
| 0.002111466 | ENSG00000115993 | O60296 | TRAK2 | trafficking kinesin protein 2 |
| 0.002111466 | ENSG00000177697 | P48509 | CD151 | CD151 molecule (Raph blood |
| | | | | group) |
| 0.002111466 | ENSG00000198792 | Q9Y519 | TMEM184B | transmembrane protein 184B |
| 0.002116685 | ENSG00000101294 | Q8TCT9 | HM13 | histocompatibility minor 13 |
| 0.002164961 | ENSG00000181027 | Q9H9S5 | FKRP | fukutin related protein |
| 0.002173662 | ENSG00000170892 | Q9BSV6 | TSEN34 | tRNA splicing endonuclease |
| | | | | subunit 34 |
| 0.00219986 | ENSG00000124145 | P31431 | SDC4 | syndecan 4 |
| 0.00221411 | ENSG00000067082 | Q99612 | KLF6 | Kruppel like factor 6 |
| 0.002214845 | ENSG00000115677 | Q00341 | HDLBP | high density lipoprotein binding |
| | | | | protein |
| 0.002242036 | ENSG00000177666 | Q96AD5 | PNPLA2 | patatin like phospholipase |
| | | | | domain containing 2 |
| 0.002243268 | ENSG00000168591 | Q71RG4 | TMUB2 | transmembrane and ubiquitin |
| | | | | like domain containing 2 |
| 0.002262953 | ENSG00000105568 | P30153 | PPP2R1A | protein phosphatase 2 scaffold |
| | | | | subunit Aalpha |
| 0.002270113 | ENSG00000127528 | Q9Y5W3 | KLF2 | Kruppel like factor 2 |
| 0.00228866 | ENSG00000138604 | O94923 | GLCE | glucuronic acid epimerase |
| 0.002290357 | ENSG00000141580 | Q5MNZ6 | WDR45B | WD repeat domain 45B |
| 0.002315811 | ENSG00000129083 | P53618 | COPB1 | coatomer protein complex |
| | | | | subunit beta 1 |
| 0.002315811 | ENSG00000171928 | Q9NYZ1 | TVP23B | trans-golgi network vesicle |
| | | | | protein 23 homolog B (S. |
| | | | | cerevisiae) |
| 0.002315811 | ENSG00000185551 | P24468 | NR2F2 | nuclear receptor subfamily 2 |
| | | | | group F member 2 |
| 0.00233337 | ENSG00000172164 | Q13884 | SNTB1 | syntrophin beta 1 |
| 0.002379885 | ENSG00000134070 | O43187 | IRAK2 | interleukin 1 receptor |
| | | | | associated kinase 2 |
| 0.002462451 | ENSG00000161714 | Q8N3E9 | PLCD3 | phospholipase C delta 3 |
| 0.002466042 | ENSG00000143570 | Q9NY26 | SLC39A1 | solute carrier family 39 member |
| | | | | 1 |
| 0.002484157 | ENSG00000139324 | Q6ZXV5 | TMTC3 | transmembrane and |
| | | | | tetratricopeptide repeat |
| | | | | containing 3 |
| 0.002659675 | ENSG00000074964 | Q9HCE6 | ARHGEF10L | Rho guanine nucleotide |
| | | | | exchange factor 10 like |
| 0.002817328 | ENSG00000110321 | P78344 | EIF4G2 | eukaryotic translation initiation |
| | | | | factor 4 gamma 2 |
| 0.002817328 | ENSG00000121989 | P27037 | ACVR2A | activin A receptor type 2A |
| 0.002817328 | ENSG00000163513 | P37173 | TGFBR2 | transforming growth factor beta |
| | | | | receptor 2 |
| 0.002833212 | ENSG00000148943 | Q9NUP9 | LIN7C | lin-7 homolog C, crumbs cell |
| | | | | polarity complex component |
| 0.002916911 | ENSG00000146376 | Q8N392 | ARHGAP18 | Rho GTPase activating protein |
| | | | | 18 |
| 0.002951981 | ENSG00000006327 | Q9NP84 | TNFRSF12A | TNF receptor superfamily |
| | | | | member 12A |
| 0.002951981 | ENSG00000160209 | O00764 | PDXK | pyridoxal (pyridoxine, vitamin |
| | | | | B6) kinase |
| 0.002992497 | ENSG00000089159 | P49023 | PXN | paxillin |
| 0.00299616 | ENSG00000183020 | O94973 | AP2A2 | adaptor related protein complex |
| | | | | 2 alpha 2 subunit |
| 0.003064027 | ENSG00000078018 | P11137 | MAP2 | microtubule associated protein |
| | | | | 2 |
| 0.003074943 | ENSG00000166333 | Q13418 | ILK | integrin linked kinase |
| 0.003170909 | ENSG00000182197 | Q16394 | EXT1 | exostosin glycosyltransferase 1 |
| 0.003170909 | ENSG00000196233 | Q96JN0 | LCOR | ligand dependent nuclear |
| | | | | receptor corepressor |
| 0.003192116 | ENSG00000058262 | P61619 | SEC61A1 | Sec61 translocon alpha 1 |
| | | | | subunit |
| 0.003192116 | ENSG00000104388 | P61019 | RAB2A | RAB2A, member RAS oncogene |
| | | | | family |
| 0.003192116 | ENSG00000112096 | P04179 | SOD2 | superoxide dismutase 2, |
| | | | | mitochondrial |
| 0.003192116 | ENSG00000168385 | Q15019 | SEPT2 | septin 2 |
| 0.00320669 | ENSG00000113194 | Q96CS3 | FAF2 | Fas associated factor family |
| | | | | member 2 |
| 0.003313704 | ENSG00000181019 | P15559 | NQO1 | NAD(P)H quinone |
| | | | | dehydrogenase 1 |
| 0.003337064 | ENSG00000159348 | Q9UHQ9 | CYB5R1 | cytochrome b5 reductase 1 |
| 0.003337064 | ENSG00000188706 | Q9Y397 | ZDHHC9 | zinc finger DHHC-type |
| | | | | containing 9 |
| 0.003458477 | ENSG00000141985 | Q99961 | SH3GL1 | SH3 domain containing GRB2 |
| | | | | like 1, endophilin A2 |
| 0.003458477 | ENSG00000182718 | P07355 | ANXA2 | annexin A2 |
| 0.003530807 | ENSG00000166275 | Q96B45 | BORCS7 | BLOC-1 related complex subunit |
| | | | | 7 |
| 0.003557286 | ENSG00000177311 | Q8NAP3 | ZBTB38 | zinc finger and BTB domain |
| | | | | containing 38 |
| 0.003619435 | ENSG00000006007 | Q9NZC3 | GDE1 | glycerophosphodiester |
| | | | | phosphodiesterase 1 |
| 0.0036748 | ENSG00000101363 | Q9NQG1 | MANBAL | mannosidase beta like |
| 0.0036748 | ENSG00000157107 | Q0JRZ9 | FCHO2 | FCH domain only 2 |
| 0.0036748 | ENSG00000170348 | P49755 | TMED10 | transmembrane p24 trafficking |
| | | | | protein 10 |
| 0.003789492 | ENSG00000151116 | Q8IX04 | UEVLD | UEV and lactate/malate |
| | | | | dehyrogenase domains |
| 0.003797788 | ENSG00000148120 | Q8N6M6 | C9orf3 | chromosome 9 open reading |
| | | | | frame 3 |
| 0.003797788 | ENSG00000185803 | Q9HAB3 | SLC52A2 | solute carrier family 52 member |
| | | | | 2 |
| 0.00385757 | ENSG00000179010 | Q9Y605 | MRFAP1 | Morf4 family associated protein |
| | | | | 1 |
| 0.003872979 | ENSG00000126458 | P10301 | RRAS | related RAS viral (r-ras) |
| | | | | oncogene homolog |
| 0.003887159 | ENSG00000106004 | P20719 | HOXA5 | homeobox A5 |
| 0.003906586 | ENSG00000039319 | Q7Z3T8 | ZFYVE16 | zinc finger FYVE-type containing |
| | | | | 16 |
| 0.003921192 | ENSG00000136810 | P10599 | TXN | thioredoxin |
| 0.003924634 | ENSG00000183963 | P53814 | SMTN | smoothelin |
| 0.003974946 | ENSG00000108774 | P51148 | RAB5C | RAB5C, member RAS oncogene |
| | | | | family |
| 0.003989269 | ENSG00000169895 | Q96A49 | SYAP1 | synapse associated protein 1 |
| 0.004025864 | ENSG00000175283 | Q9UPQ8 | DOLK | dolichol kinase |
| 0.004035904 | ENSG00000171302 | Q8WVQ1 | CANT1 | calcium activated nucleotidase 1 |
| 0.004095154 | ENSG00000120697 | Q9Y673 | ALG5 | ALG5, dolichyl-phosphate beta- |
| | | | | glucosyltransferase |
| 0.004140176 | ENSG00000185475 | Q7Z7N9 | TMEM179B | transmembrane protein 179B |
| 0.004140925 | ENSG00000107331 | Q9BZC7 | ABCA2 | ATP binding cassette subfamily |
| | | | | A member 2 |
| 0.004211967 | ENSG00000119673 | P49753 | ACOT2 | acyl-CoA thioesterase 2 |
| 0.004234126 | ENSG00000034510 | P63313 | TMSB10 | thymosin beta 10 |
| 0.004247695 | ENSG00000133466 | Q9BXI9 | C1QTNF6 | C1q and tumor necrosis factor |
| | | | | related protein 6 |
| 0.004247695 | ENSG00000164056 | O43609 | SPRY1 | sprouty RTK signaling antagonist |
| | | | | 1 |
| 0.004258967 | ENSG00000198668 | P62158 | CALM1 | calmodulin 1 |
| 0.004325126 | ENSG00000186174 | Q86UU0 | BCL9L | B-cell CLL/lymphoma 9-like |
| 0.004340802 | ENSG00000133816 | O94851 | MICAL2 | microtubule associated |
| | | | | monooxygenase, calponin and |
| | | | | LIM domain containing 2 |
| 0.004442095 | ENSG00000115504 | Q8NDI1 | EHBP1 | EH domain binding protein 1 |
| 0.004453185 | ENSG00000013375 | O95394 | PGM3 | phosphoglucomutase 3 |
| 0.004453185 | ENSG00000141367 | Q00610 | CLTC | clathrin heavy chain |
| 0.004453185 | ENSG00000151689 | P49441 | INPP1 | inositol polyphosphate-1- |
| | | | | phosphatase |
| 0.004459548 | ENSG00000083312 | Q92973 | TNPO1 | transportin 1 |
| 0.004462125 | ENSG00000196586 | Q9UM54 | MYO6 | myosin VI |
| 0.004483048 | ENSG00000164976 | Q6NSJ0 | KIAA1161 | KIAA1161 |
| 0.004551507 | ENSG00000150093 | P05556 | ITGB1 | integrin subunit beta 1 |
| 0.004552203 | ENSG00000204070 | Q8N2H4 | SYS1 | Sys1 golgi trafficking protein |
| 0.004665073 | ENSG00000136717 | O00499 | BIN1 | bridging integrator 1 |
| 0.004679369 | ENSG00000171174 | Q9H477 | RBKS | ribokinase |
| 0.004725308 | ENSG00000135404 | P08962 | CD63 | CD63 molecule |
| 0.004791373 | ENSG00000134910 | P46977 | STT3A | STT3A, catalytic subunit of the |
| | | | | oligosaccharyltransferase |
| | | | | complex |
| 0.004846953 | ENSG00000104635 | Q15043 | SLC39A14 | solute carrier family 39 member |
| | | | | 14 |
| 0.004846953 | ENSG00000164494 | Q86YH6 | PDSS2 | prenyl (decaprenyl) diphosphate |
| | | | | synthase, subunit 2 |
| 0.005158254 | ENSG00000143553 | O95295 | SNAPIN | SNAP associated protein |
| 0.005293793 | ENSG00000198898 | P47755 | CAPZA2 | capping actin protein of muscle |
| | | | | Z-line alpha subunit 2 |
| 0.005318882 | ENSG00000086598 | Q15363 | TMED2 | transmembrane p24 trafficking |
| | | | | protein 2 |
| 0.005440162 | ENSG00000072803 | Q9UKB1 | FBXW11 | F-box and WD repeat domain |
| | | | | containing 11 |
| 0.005447597 | ENSG00000001036 | Q9BTY2 | FUCA2 | fucosidase, alpha-L- 2, plasma |
| 0.005461355 | ENSG00000165169 | P51808 | DYNLT3 | dynein light chain Tctex-type 3 |
| 0.005472678 | ENSG00000108946 | P10644 | PRKAR1A | protein kinase cAMP-dependent |
| | | | | type I regulatory subunit alpha |
| 0.005472678 | ENSG00000144040 | Q8TD22 | SFXN5 | sideroflexin 5 |
| 0.005512283 | ENSG00000138760 | Q14108 | SCARB2 | scavenger receptor class B |
| | | | | member 2 |
| 0.005538044 | ENSG00000070540 | Q5MNZ9 | WIPI1 | WD repeat domain, |
| | | | | phosphoinositide interacting 1 |
| 0.005613215 | ENSG00000135720 | O43237 | DYNC1LI2 | dynein cytoplasmic 1 light |
| | | | | intermediate chain 2 |
| 0.005635895 | ENSG00000127948 | P16435 | POR | cytochrome p450 |
| | | | | oxidoreductase |
| 0.005701934 | ENSG00000151348 | Q93063 | EXT2 | exostosin glycosyltransferase 2 |
| 0.005730165 | ENSG00000134371 | Q6P1J9 | CDC73 | cell division cycle 73 |
| 0.005765756 | ENSG00000164111 | P08758 | ANXA5 | annexin A5 |
| 0.005772183 | ENSG00000099282 | O95858 | TSPAN15 | tetraspanin 15 |
| 0.005778921 | ENSG00000108679 | Q08380 | LGALS3BP | galectin 3 binding protein |
| 0.005800162 | ENSG00000171155 | Q96EU7 | C1GALT1C1 | C1GALT1 specific chaperone 1 |
| 0.005979858 | ENSG00000068903 | Q8IXJ6 | SIRT2 | sirtuin 2 |
| 0.00607531 | ENSG00000189077 | Q9BXJ8 | TMEM120A | transmembrane protein 120A |
| 0.006131227 | ENSG00000107651 | Q9Y6Y8 | SEC23IP | SEC23 interacting protein |
| 0.006144096 | ENSG00000144802 | Q9BYH8 | NFKBIZ | NFKB inhibitor zeta |
| 0.006151122 | ENSG00000129235 | Q9BRA2 | TXNDC17 | thioredoxin domain containing |
| | | | | 17 |
| 0.006160011 | ENSG00000155876 | Q7L523 | RRAGA | Ras related GTP binding A |
| 0.006170101 | ENSG00000204673 | Q96B36 | AKT1S1 | AKT1 substrate 1 |
| 0.006207312 | ENSG00000127947 | Q05209 | PTPN12 | protein tyrosine phosphatase, |
| | | | | non-receptor type 12 |
| 0.006207312 | ENSG00000140416 | P09493 | TPM1 | tropomyosin 1 (alpha) |
| 0.006383356 | ENSG00000197102 | Q14204 | DYNC1H1 | dynein cytoplasmic 1 heavy |
| | | | | chain 1 |
| 0.006438067 | ENSG00000161677 | Q8TAC2 | JOSD2 | Josephin domain containing 2 |
| 0.006438157 | ENSG00000166794 | P23284 | PPIB | peptidylprolyl isomerase B |
| 0.006484601 | ENSG00000120306 | Q9H1C7 | CYSTM1 | cysteine rich transmembrane |
| | | | | module containing 1 |
| 0.006512936 | ENSG00000145555 | Q9HD67 | MYO10 | myosin X |
| 0.006592676 | ENSG00000035403 | P18206 | VCL | vinculin |
| 0.006618222 | ENSG00000169599 | Q9UMS0 | NFU1 | NFU1 iron-sulfur cluster scaffold |
| 0.006647021 | ENSG00000221869 | P49716 | CEBPD | CCAAT/enhancer binding |
| | | | | protein delta |
| 0.00677926 | ENSG00000163110 | Q96HC4 | PDLIM5 | PDZ and LIM domain 5 |
| 0.006846275 | ENSG00000117528 | P28288 | ABCD3 | ATP binding cassette subfamily |
| | | | | D member 3 |
| 0.00686206 | ENSG00000145730 | P19021 | PAM | peptidylglycine alpha-amidating |
| | | | | monooxygenase |
| 0.006895127 | ENSG00000172239 | Q9H074 | PAIP1 | poly(A) binding protein |
| | | | | interacting protein 1 |
| 0.006938032 | ENSG00000141551 | P48730 | CSNK1D | casein kinase 1 delta |
| 0.007003547 | ENSG00000182621 | Q9NQ66 | PLCB1 | phospholipase C beta 1 |
| 0.00706737 | ENSG00000107175 | O43889 | CREB3 | cAMP responsive element |
| | | | | binding protein 3 |
| 0.00706737 | ENSG00000157224 | P56749 | CLDN12 | claudin 12 |
| 0.00706737 | ENSG00000187240 | Q8NCM8 | DYNC2H1 | dynein cytoplasmic 2 heavy |
| | | | | chain 1 |
| 0.00707127 | ENSG00000152818 | P46939 | UTRN | utrophin |
| 0.007101997 | ENSG00000096070 | Q9ULD4 | BRPF3 | bromodomain and PHD finger |
| | | | | containing 3 |
| 0.007224767 | ENSG00000118705 | P04844 | RPN2 | ribophorin II |
| 0.007246731 | ENSG00000110442 | Q9P000 | COMMD9 | COMM domain containing 9 |
| 0.007247876 | ENSG00000162616 | Q9UDY4 | DNAJB4 | DnaJ heat shock protein family |
| | | | | (Hsp40) member B4 |
| 0.007247876 | ENSG00000162909 | P17655 | CAPN2 | calpain 2 |
| 0.007267976 | ENSG00000181830 | Q96A29 | SLC35C1 | solute carrier family 35 member |
| | | | | C1 |
| 0.007282991 | ENSG00000173692 | Q99460 | PSMD1 | proteasome 26S subunit, non- |
| | | | | ATPase 1 |
| 0.007293315 | ENSG00000063322 | Q9NX70 | MED29 | mediator complex subunit 29 |
| 0.007400154 | ENSG00000196812 | Q9H4T2 | ZSCAN16 | zinc finger and SCAN domain |
| | | | | containing 16 |
| 0.007425277 | ENSG00000143554 | Q5K4L6 | SLC27A3 | solute carrier family 27 member |
| | | | | 3 |
| 0.007446483 | ENSG00000072849 | Q9GZP9 | DERL2 | derlin 2 |
| 0.007446483 | ENSG00000132002 | P25685 | DNAJB1 | DnaJ heat shock protein family |
| | | | | (Hsp40) member B1 |
| 0.007447292 | ENSG00000131446 | P26572 | MGAT1 | mannosyl (alpha-1,3-)- |
| | | | | glycoprotein beta-1,2-N- |
| | | | | acetylglucosaminyltransferase |
| 0.007482852 | ENSG00000184840 | Q9BVK6 | TMED9 | transmembrane p24 trafficking |
| | | | | protein 9 |
| 0.007486567 | ENSG00000138814 | Q08209 | PPP3CA | protein phosphatase 3 catalytic |
| | | | | subunit alpha |
| 0.00752626 | ENSG00000013275 | P43686 | PSMC4 | proteasome 26S subunit, |
| | | | | ATPase 4 |
| 0.00752626 | ENSG00000129255 | O75352 | MPDU1 | mannose-P-dolichol utilization |
| | | | | defect 1 |
| 0.007573945 | ENSG00000085978 | Q676U5 | ATG16L1 | autophagy related 16 like 1 |
| 0.007600793 | ENSG00000143924 | Q9HC35 | EML4 | echinoderm microtubule |
| | | | | associated protein like 4 |
| 0.00760532 | ENSG00000062716 | Q96GC9 | VMP1 | vacuole membrane protein 1 |
| 0.007643752 | ENSG00000198431 | Q16881 | TXNRD1 | thioredoxin reductase 1 |
| 0.007659913 | ENSG00000108854 | Q9HAU4 | SMURF2 | SMAD specific E3 ubiquitin |
| | | | | protein ligase 2 |
| 0.007755433 | ENSG00000144591 | Q96IJ6 | GMPPA | GDP-mannose |
| | | | | pyrophosphorylase A |
| 0.00779976 | ENSG00000135930 | O60573 | EIF4E2 | eukaryotic translation initiation |
| | | | | factor 4E family member 2 |
| 0.007825725 | ENSG00000124788 | P54253 | ATXN1 | ataxin 1 |
| 0.007979329 | ENSG00000132842 | O00203 | AP3B1 | adaptor related protein complex |
| | | | | 3 beta 1 subunit |
| 0.008029342 | ENSG00000196526 | Q8N556 | AFAP1 | actin filament associated |
| | | | | protein 1 |
| 0.008056183 | ENSG00000181789 | Q9Y678 | COPG1 | coatomer protein complex |
| | | | | subunit gamma 1 |
| 0.008141941 | ENSG00000136159 | Q9NV35 | NUDT15 | nudix hydrolase 15 |
| 0.008141941 | ENSG00000139132 | Q96M96 | FGD4 | FYVE, RhoGEF and PH domain |
| | | | | containing 4 |
| 0.00816036 | ENSG00000066322 | Q9BW60 | ELOVL1 | ELOVL fatty acid elongase 1 |
| 0.008261872 | ENSG00000137414 | Q9UBU6 | FAM8A1 | family with sequence similarity |
| | | | | 8 member A1 |
| 0.00831478 | ENSG00000129625 | Q00765 | REEP5 | receptor accessory protein 5 |
| 0.008476528 | ENSG00000121940 | Q96S66 | CLCC1 | chloride channel CLIC like 1 |
| 0.008629403 | ENSG00000117899 | Q14696 | MESDC2 | mesoderm development |
| | | | | candidate 2 |
| 0.008629403 | ENSG00000213859 | Q693B1 | KCTD11 | potassium channel |
| | | | | tetramerization domain |
| | | | | containing 11 |
| 0.008675965 | ENSG00000122958 | O75436 | VPS26A | VPS26, retromer complex |
| | | | | component A |
| 0.008751917 | ENSG00000134686 | Q8IXK0 | PHC2 | polyhomeotic homolog 2 |
| 0.00876693 | ENSG00000107185 | Q92546 | RGP1 | RGP1 homolog, RAB6A GEF |
| | | | | complex partner 1 |
| 0.008933988 | ENSG00000109016 | Q6IAN0 | DHRS7B | dehydrogenase/reductase 7B |
| 0.00900759 | ENSG00000129219 | O14939 | PLD2 | phospholipase D2 |
| 0.00904335 | ENSG00000108588 | Q96A33 | CCDC47 | coiled-coil domain containing 47 |
| 0.009054464 | ENSG00000112977 | P51397 | DAP | death associated protein |
| 0.009068109 | ENSG00000127952 | Q9Y6J8 | STYXL1 | serine/threonine/tyrosine |
| | | | | interacting like 1 |
| 0.009068109 | ENSG00000178996 | Q96RF0 | SNX18 | sorting nexin 18 |
| 0.00908098 | ENSG00000180185 | Q6P587 | FAHD1 | fumarylacetoacetate hydrolase |
| | | | | domain containing 1 |
| 0.009174223 | ENSG00000164924 | P63104 | YWHAZ | tyrosine 3- |
| | | | | monooxygenase/tryptophan 5- |
| | | | | monooxygenase activation |
| | | | | protein zeta |
| 0.009287626 | ENSG00000129474 | Q96IF1 | AJUBA | ajuba LIM protein |
| 0.009341506 | ENSG00000169047 | P35568 | IRS1 | insulin receptor substrate 1 |
| 0.009621537 | ENSG00000213977 | O14907 | TAX1BP3 | Tax1 binding protein 3 |
| 0.009677337 | ENSG00000078902 | Q9H0E2 | TOLLIP | toll interacting protein |
| 0.009724424 | ENSG00000198162 | O60476 | MAN1A2 | mannosidase alpha class 1A |
| | | | | member 2 |
| 0.009760083 | ENSG00000168374 | P18085 | ARF4 | ADP ribosylation factor 4 |
| 0.009782913 | ENSG00000120509 | Q5EBL8 | PDZD11 | PDZ domain containing 11 |
| 0.009812181 | ENSG00000264522 | Q6GQQ9 | OTUD7B | OTU deubiquitinase 7B |
| 0.009822409 | ENSG00000138459 | Q9BS91 | SLC35A5 | solute carrier family 35 member |
| | | | | A5 |
| 0.009896619 | ENSG00000102595 | Q9NYU1 | UGGT2 | UDP-glucose glycoprotein |
| | | | | glucosyltransferase 2 |
| 0.009949541 | ENSG00000148180 | P06396 | GSN | gelsolin |
| 0.009950261 | ENSG00000100522 | Q96EK6 | GNPNAT1 | glucosamine-phosphate N- |
| | | | | acetyltransferase 1 |
| 0.009954521 | ENSG00000112210 | Q9ULC3 | RAB23 | RAB23, member RAS oncogene |
| | | | | family |
| 0.009966104 | ENSG00000006468 | P50549 | ETV1 | ETS variant 1 |
| 0.009966104 | ENSG00000049245 | Q15836 | VAMP3 | vesicle associated membrane |
| | | | | protein 3 |
| 0.009966104 | ENSG00000100994 | P11216 | PYGB | phosphorylase, glycogen; brain |
| 0.009966104 | ENSG00000106546 | P35869 | AHR | aryl hydrocarbon receptor |
| 0.009966104 | ENSG00000127884 | P30084 | ECHS1 | enoyl-CoA hydratase, short |
| | | | | chain 1 |
| 0.009966104 | ENSG00000137501 | Q9HCH5 | SYTL2 | synaptotagmin like 2 |
| 0.009966104 | ENSG00000166471 | Q5BJD5 | TMEM41B | transmembrane protein 41B |
| 0.010019458 | ENSG00000177888 | Q5SVQ8 | ZBTB41 | zinc finger and BTB domain |
| | | | | containing 41 |
| 0.01017607 | ENSG00000177542 | Q9H936 | SLC25A22 | solute carrier family 25 member |
| | | | | 22 |
| 0.010234301 | ENSG00000123728 | Q9Y3L5 | RAP2C | RAP2C, member of RAS |
| | | | | oncogene family |
| 0.010234301 | ENSG00000149257 | P50454 | SERPINH1 | serpin family H member 1 |
| 0.010234301 | ENSG00000169032 | Q02750 | MAP2K1 | mitogen-activated protein |
| | | | | kinase kinase 1 |
| 0.010234301 | ENSG00000169490 | Q9BX73 | TM2D2 | TM2 domain containing 2 |
| 0.010305004 | ENSG00000129473 | Q92843 | BCL2L2 | BCL2 like 2 |
| 0.010401442 | ENSG00000168916 | Q9ULD9 | ZNF608 | zinc finger protein 608 |
| 0.010490477 | ENSG00000119471 | Q6YN16 | HSDL2 | hydroxysteroid dehydrogenase |
| | | | | like 2 |
| 0.010645838 | ENSG00000157181 | Q5SWX8 | C1orf27 | chromosome 1 open reading |
| | | | | frame 27 |
| 0.010731782 | ENSG00000101150 | O43399 | TPD52L2 | tumor protein D52 like 2 |
| 0.010731782 | ENSG00000139178 | Q9NZP8 | C1RL | complement C1r subcomponent |
| | | | | like |
| 0.010747345 | ENSG00000112419 | O75167 | PHACTR2 | phosphatase and actin regulator |
| | | | | 2 |
| 0.010804111 | ENSG00000106868 | Q6UWL2 | SUSD1 | sushi domain containing 1 |
| 0.010941202 | ENSG00000171150 | O75159 | SOCS5 | suppressor of cytokine signaling |
| | | | | 5 |
| 0.010953221 | ENSG00000115425 | Q9BY49 | PECR | peroxisomal trans-2-enoyl-CoA |
| | | | | reductase |
| 0.01095589 | ENSG00000079308 | Q9HBL0 | TNS1 | tensin 1 |
| 0.01100201 | ENSG00000155366 | P08134 | RHOC | ras homolog family member C |
| 0.011004725 | ENSG00000128311 | Q16762 | TST | thiosulfate sulfurtransferase |
| 0.011055498 | ENSG00000143183 | Q9UM00 | TMCO1 | transmembrane and coiled-coil |
| | | | | domains 1 |
| 0.011064708 | ENSG00000047230 | Q9NRF8 | CTPS2 | CTP synthase 2 |
| 0.01109893 | ENSG00000103266 | Q9UNE7 | STUB1 | STIP1 homology and U-box |
| | | | | containing protein 1 |
| 0.01109893 | ENSG00000204147 | P0C7U1 | ASAH2B | N-acylsphingosine |
| | | | | amidohydrolase 2B |
| 0.011175054 | ENSG00000042445 | Q6NUM9 | RETSAT | retinol saturase |
| 0.011175054 | ENSG00000126062 | Q12893 | TMEM115 | transmembrane protein 115 |
| 0.011434462 | ENSG00000128512 | Q8N1I0 | DOCK4 | dedicator of cytokinesis 4 |
| 0.011500509 | ENSG00000179604 | Q9H3Q1 | CDC42EP4 | CDC42 effector protein 4 |
| 0.011525848 | ENSG00000025434 | Q13133 | NR1H3 | nuclear receptor subfamily 1 |
| | | | | group H member 3 |
| 0.011530393 | ENSG00000008086 | O76039 | CDKL5 | cyclin dependent kinase like 5 |
| 0.011550319 | ENSG00000167004 | P30101 | PDIA3 | protein disulfide isomerase |
| | | | | family A member 3 |
| 0.011559918 | ENSG00000166224 | O95470 | SGPL1 | sphingosine-1-phosphate lyase |
| | | | | 1 |
| 0.011559918 | ENSG00000184277 | Q9BRN9 | TM2D3 | TM2 domain containing 3 |
| 0.01157058 | ENSG00000134243 | Q99523 | SORT1 | sortilin 1 |
| 0.011593698 | ENSG00000103042 | Q9NVC3 | SLC38A7 | solute carrier family 38 member |
| | | | | 7 |
| 0.011640158 | ENSG00000118515 | O00141 | SGK1 | serum/glucocorticoid regulated |
| | | | | kinase 1 |
| 0.011640158 | ENSG00000124570 | P35237 | SERPINB6 | serpin family B member 6 |
| 0.011659753 | ENSG00000168575 | Q08357 | SLC20A2 | solute carrier family 20 member |
| | | | | 2 |
| 0.011674712 | ENSG00000174851 | O95070 | YIF1A | Yip1 interacting factor homolog |
| | | | | A, membrane trafficking protein |
| 0.011684275 | ENSG00000151292 | Q9Y6M4 | CSNK1G3 | casein kinase 1 gamma 3 |
| 0.011684275 | ENSG00000167996 | P02794 | FTH1 | ferritin heavy chain 1 |
| 0.011684275 | ENSG00000181904 | Q7Z6I8 | C5orf24 | chromosome 5 open reading |
| | | | | frame 24 |
| 0.011690405 | ENSG00000129562 | P61803 | DAD1 | defender against cell death 1 |
| 0.011811614 | ENSG00000185716 | Q8NHV5 | C16orf52 | chromosome 16 open reading |
| | | | | frame 52 |
| 0.01189872 | ENSG00000205339 | O95373 | IPO7 | importin 7 |
| 0.012030824 | ENSG00000166908 | Q8TBX8 | PIP4K2C | phosphatidylinositol-5- |
| | | | | phosphate 4-kinase type 2 |
| | | | | gamma |
| 0.012038479 | ENSG00000101474 | Q9HDC9 | APMAP | adipocyte plasma membrane |
| | | | | associated protein |
| 0.012047644 | ENSG00000197747 | P60903 | S100A10 | S100 calcium binding protein |
| | | | | A10 |
| 0.012055823 | ENSG00000049449 | Q15293 | RCN1 | reticulocalbin 1 |
| 0.012055823 | ENSG00000136856 | Q9NY64 | SLC2A8 | solute carrier family 2 member |
| | | | | 8 |
| 0.012355985 | ENSG00000150403 | Q6UWJ1 | TMCO3 | transmembrane and coiled-coil |
| | | | | domains 3 |
| 0.01241916 | ENSG00000137936 | O75815 | BCAR3 | breast cancer anti-estrogen |
| | | | | resistance 3 |
| 0.012423864 | ENSG00000138069 | P62820 | RAB1A | RAB1A, member RAS oncogene |
| | | | | family |
| 0.012429142 | ENSG00000179933 | Q9NWQ9 | C14orf119 | chromosome 14 open reading |
| | | | | frame 119 |
| 0.012471578 | ENSG00000080815 | P49768 | PSEN1 | presenilin 1 |
| 0.012471578 | ENSG00000103502 | O14735 | CDIPT | CDP-diacylglycerol--inositol 3- |
| | | | | phosphatidyltransferase |
| 0.012471578 | ENSG00000180611 | Q8IYB1 | MB21D2 | Mab-21 domain containing 2 |
| 0.012529082 | ENSG00000116539 | Q9NR48 | ASH1L | ASH1 like histone lysine |
| | | | | methyltransferase |
| 0.012558707 | ENSG00000170525 | Q16875 | PFKFB3 | 6-phosphofructo-2- |
| | | | | kinase/fructose-2,6- |
| | | | | biphosphatase 3 |
| 0.012593766 | ENSG00000073803 | O43283 | MAP3K13 | mitogen-activated protein |
| | | | | kinase kinase kinase 13 |
| 0.012711645 | ENSG00000166311 | P17405 | SMPD1 | sphingomyelin |
| | | | | phosphodiesterase 1 |
| 0.012830627 | ENSG00000185728 | Q7Z739 | YTHDF3 | YTH N6-methyladenosine RNA |
| | | | | binding protein 3 |
| 0.012883685 | ENSG00000129493 | Q86XA9 | HEATR5A | HEAT repeat containing 5A |
| 0.012999993 | ENSG00000135766 | Q9GZT9 | EGLN1 | egl-9 family hypoxia inducible |
| | | | | factor 1 |
| 0.013363951 | ENSG00000116741 | P41220 | RGS2 | regulator of G-protein signaling |
| | | | | 2 |
| 0.013363951 | ENSG00000126247 | P04632 | CAPNS1 | calpain small subunit 1 |
| 0.013467673 | ENSG00000115170 | Q04771 | ACVR1 | activin A receptor type 1 |
| 0.013490962 | ENSG00000105429 | Q7Z7M0 | MEGF8 | multiple EGF like domains 8 |
| 0.013561047 | ENSG00000197713 | Q96AT9 | RPE | ribulose-5-phosphate-3- |
| | | | | epimerase |
| 0.013565455 | ENSG00000127774 | Q9BV81 | EMC6 | ER membrane protein complex |
| | | | | subunit 6 |
| 0.013648502 | ENSG00000100997 | Q8N2K0 | ABHD12 | abhydrolase domain containing |
| | | | | 12 |
| 0.013648502 | ENSG00000147400 | P41208 | CETN2 | centrin 2 |
| 0.013682834 | ENSG00000157483 | Q12965 | MYO1E | myosin IE |
| 0.013771607 | ENSG00000162695 | Q8NEW0 | SLC30A7 | solute carrier family 30 member |
| | | | | 7 |
| 0.013817089 | ENSG00000181523 | P51688 | SGSH | N-sulfoglucosamine |
| | | | | sulfohydrolase |
| 0.013913115 | ENSG00000171067 | Q96F05 | C11orf24 | chromosome 11 open reading |
| | | | | frame 24 |
| 0.014043739 | ENSG00000068697 | Q15012 | LAPTM4A | lysosomal protein |
| | | | | transmembrane 4 alpha |
| 0.014097114 | ENSG00000171867 | P04156 | PRNP | prion protein |
| 0.014108578 | ENSG00000136238 | P63000 | RAC1 | ras-related C3 botulinum toxin |
| | | | | substrate 1 (rho family, small |
| | | | | GTP binding protein Rac1) |
| 0.014118402 | ENSG00000157933 | P12755 | SKI | SKI proto-oncogene |
| 0.014118402 | ENSG00000174903 | Q9H0U4 | RAB1B | RAB1B, member RAS oncogene |
| | | | | family |
| 0.014192376 | ENSG00000214063 | O14817 | TSPAN4 | tetraspanin 4 |
| 0.014196977 | ENSG00000182154 | Q8IXM3 | MRPL41 | mitochondrial ribosomal protein |
| | | | | L41 |
| 0.014237851 | ENSG00000102471 | Q9NV92 | NDFIP2 | Nedd4 family interacting protein |
| | | | | 2 |
| 0.014366602 | ENSG00000110628 | Q96BI1 | SLC22A18 | solute carrier family 22 member |
| | | | | 18 |
| 0.014545782 | ENSG00000090054 | O15269 | SPTLC1 | serine palmitoyltransferase long |
| | | | | chain base subunit 1 |
| 0.014545782 | ENSG00000137185 | O15535 | ZSCAN9 | zinc finger and SCAN domain |
| | | | | containing 9 |
| 0.014581231 | ENSG00000172725 | Q9BR76 | CORO1B | coronin 1B |
| 0.014725173 | ENSG00000099341 | P48556 | PSMD8 | proteasome 26S subunit, non- |
| | | | | ATPase 8 |
| 0.014728832 | ENSG00000126903 | P09131 | SLC10A3 | solute carrier family 10 member |
| | | | | 3 |
| 0.01483616 | ENSG00000011566 | Q8IVH8 | MAP4K3 | mitogen-activated protein |
| | | | | kinase kinase kinase kinase 3 |
| 0.014908883 | ENSG00000091409 | P23229 | ITGA6 | integrin subunit alpha 6 |
| 0.015251681 | ENSG00000172428 | Q8WXC6 | COPS9 | COP9 signalosome subunit 9 |
| 0.015384001 | ENSG00000166016 | Q8N961 | ABTB2 | ankyrin repeat and BTB domain |
| | | | | containing 2 |
| 0.015630439 | ENSG00000121073 | P78383 | SLC35B1 | solute carrier family 35 member |
| | | | | B1 |
| 0.015747153 | ENSG00000072210 | P51648 | ALDH3A2 | aldehyde dehydrogenase 3 |
| | | | | family member A2 |
| 0.015835353 | ENSG00000114744 | Q86X83 | COMMD2 | COMM domain containing 2 |
| 0.015878882 | ENSG00000106636 | O15498 | YKT6 | YKT6 v-SNARE homolog (S. |
| | | | | cerevisiae) |
| 0.016175759 | ENSG00000119912 | P14735 | IDE | insulin degrading enzyme |
| 0.016175759 | ENSG00000180398 | Q8NI22 | MCFD2 | multiple coagulation factor |
| | | | | deficiency 2 |
| 0.016183189 | ENSG00000183864 | Q14106 | TOB2 | transducer of ERBB2, 2 |
| 0.016286058 | ENSG00000118200 | Q08AD1 | CAMSAP2 | calmodulin regulated spectrin |
| | | | | associated protein family |
| | | | | member 2 |
| 0.016430544 | ENSG00000170145 | Q9H0K1 | SIK2 | salt inducible kinase 2 |
| 0.016549491 | ENSG00000132356 | Q13131 | PRKAA1 | protein kinase AMP-activated |
| | | | | catalytic subunit alpha 1 |
| 0.01660753 | ENSG00000142507 | P28072 | PSMB6 | proteasome subunit beta 6 |
| 0.016709025 | ENSG00000144043 | Q6UWH6 | TEX261 | testis expressed 261 |
| 0.016785271 | ENSG00000170385 | Q9Y6M5 | SLC30A1 | solute carrier family 30 member |
| | | | | 1 |
| 0.016829124 | ENSG00000117592 | P30041 | PRDX6 | peroxiredoxin 6 |
| 0.016833404 | ENSG00000104853 | O96005 | CLPTM1 | CLPTM1, transmembrane |
| | | | | protein |
| 0.016889785 | ENSG00000146433 | Q9P2C4 | TMEM181 | transmembrane protein 181 |
| 0.01689626 | ENSG00000153113 | P20810 | CAST | calpastatin |
| 0.016911197 | ENSG00000100804 | P28074 | PSMB5 | proteasome subunit beta 5 |
| 0.017014531 | ENSG00000068971 | Q15173 | PPP2R5B | protein phosphatase 2 |
| | | | | regulatory subunit B′beta |
| 0.017033892 | ENSG00000124214 | O95793 | STAU1 | staufen double-stranded RNA |
| | | | | binding protein 1 |
| 0.017088783 | ENSG00000185825 | P51572 | BCAP31 | B-cell receptor-associated |
| | | | | protein 31 |
| 0.017094556 | ENSG00000044115 | P35221 | CTNNA1 | catenin alpha 1 |
| 0.017141919 | ENSG00000196975 | P09525 | ANXA4 | annexin A4 |
| 0.017181962 | ENSG00000115350 | Q9NR33 | POLE4 | DNA polymerase epsilon 4, |
| | | | | accessory subunit |
| 0.017213327 | ENSG00000242372 | P56537 | EIF6 | eukaryotic translation initiation |
| | | | | factor 6 |
| 0.01731812 | ENSG00000177239 | Q9UKM7 | MAN1B1 | mannosidase alpha class 1B |
| | | | | member 1 |
| 0.01735504 | ENSG00000183255 | P53801 | PTTG1IP | pituitary tumor-transforming 1 |
| | | | | interacting protein |
| 0.01739368 | ENSG00000109066 | Q8NE00 | TMEM104 | transmembrane protein 104 |
| 0.01739368 | ENSG00000168216 | Q9NUN5 | LMBRD1 | LMBR1 domain containing 1 |
| 0.01741619 | ENSG00000136143 | Q9P2R7 | SUCLA2 | succinate-CoA ligase ADP- |
| | | | | forming beta subunit |
| 0.017491394 | ENSG00000175348 | Q9NQ34 | TMEM9B | TMEM9 domain family member |
| | | | | B |
| 0.017526569 | ENSG00000122592 | P31268 | HOXA7 | homeobox A7 |
| 0.01758774 | ENSG00000130827 | P51805 | PLXNA3 | plexin A3 |
| 0.01758774 | ENSG00000145354 | Q8N5K1 | CISD2 | CDGSH iron sulfur domain 2 |
| 0.017659666 | ENSG00000174775 | P01112 | HRAS | HRas proto-oncogene, GTPase |
| 0.01775569 | ENSG00000108582 | O75976 | CPD | carboxypeptidase D |
| 0.017780594 | ENSG00000070404 | O95633 | FSTL3 | follistatin like 3 |
| 0.017850847 | ENSG00000164713 | O95415 | BRI3 | brain protein I3 |
| 0.017888231 | ENSG00000122042 | O95164 | UBL3 | ubiquitin like 3 |
| 0.017932434 | ENSG00000148358 | Q5VW38 | GPR107 | G protein-coupled receptor 107 |
| 0.018105806 | ENSG00000145919 | Q96IK1 | BOD1 | biorientation of chromosomes |
| | | | | in cell division 1 |
| 0.018192352 | ENSG00000213463 | P57105 | SYNJ2BP | synaptojanin 2 binding protein |
| 0.01823704 | ENSG00000102804 | Q15714 | TSC22D1 | TSC22 domain family member 1 |
| 0.018345629 | ENSG00000116209 | Q9BXS4 | TMEM59 | transmembrane protein 59 |
| 0.018457068 | ENSG00000140396 | Q15596 | NCOA2 | nuclear receptor coactivator 2 |
| 0.018460479 | ENSG00000113790 | Q08426 | EHHADH | enoyl-CoA, hydratase/3- |
| | | | | hydroxyacyl CoA dehydrogenase |
| 0.018679845 | ENSG00000123562 | Q15014 | MORF4L2 | mortality factor 4 like 2 |
| 0.018754878 | ENSG00000206527 | Q6Y1H2 | HACD2 | 3-hydroxyacyl-CoA dehydratase |
| | | | | 2 |
| 0.018755866 | ENSG00000124920 | Q9Y2G1 | MYRF | myelin regulatory factor |
| 0.018844328 | ENSG00000094975 | Q9UBS9 | SUCO | SUN domain containing |
| | | | | ossification factor |
| 0.018844328 | ENSG00000260027 | P09629 | HOXB7 | homeobox B7 |
| 0.018887254 | ENSG00000198561 | O60716 | CTNND1 | catenin delta 1 |
| 0.018926259 | ENSG00000147592 | Q53H82 | LACTB2 | lactamase beta 2 |
| 0.018956482 | ENSG00000152684 | Q9BRX2 | PELO | pelota homolog (Drosophila) |
| 0.019001861 | ENSG00000243147 | O75394 | MRPL33 | mitochondrial ribosomal protein |
| | | | | L33 |
| 0.019055523 | ENSG00000134982 | P25054 | APC | APC, WNT signaling pathway |
| | | | | regulator |
| 0.019135552 | ENSG00000135845 | Q92535 | PIGC | phosphatidylinositol glycan |
| | | | | anchor biosynthesis class C |
| 0.019147004 | ENSG00000163902 | P04843 | RPN1 | ribophorin I |
| 0.019186263 | ENSG00000116044 | Q16236 | NFE2L2 | nuclear factor, erythroid 2 like 2 |
| 0.01927509 | ENSG00000241685 | Q92747 | ARPC1A | actin related protein 2/3 |
| | | | | complex subunit 1A |
| 0.019447367 | ENSG00000221886 | Q8IZ13 | ZBED8 | zinc finger BED-type containing |
| | | | | 8 |
| 0.019471605 | ENSG00000115159 | P43304 | GPD2 | glycerol-3-phosphate |
| | | | | dehydrogenase 2 |
| 0.019471605 | ENSG00000119318 | P54727 | RAD23B | RAD23 homolog B, nucleotide |
| | | | | excision repair protein |
| 0.019528295 | ENSG00000145545 | P18405 | SRD5A1 | steroid 5 alpha-reductase 1 |
| 0.019846725 | ENSG00000179630 | Q8IV20 | LACC1 | laccase domain containing 1 |
| 0.019875884 | ENSG00000110330 | Q13490 | BIRC2 | baculoviral IAP repeat |
| | | | | containing 2 |
| 0.019905895 | ENSG00000074842 | Q969H8 | MYDGF | myeloid derived growth factor |
| 0.019968474 | ENSG00000136295 | Q9C0H2 | TTYH3 | tweety family member 3 |
| 0.020024848 | ENSG00000127837 | Q13685 | AAMP | angio associated migratory cell |
| | | | | protein |
| 0.020024848 | ENSG00000159063 | Q9BVK2 | ALG8 | ALG8, alpha-1,3- |
| | | | | glucosyltransferase |
| 0.020068747 | ENSG00000066455 | Q8TBA6 | GOLGA5 | golgin A5 |
| 0.020243294 | ENSG00000149292 | Q9H892 | TTC12 | tetratricopeptide repeat domain |
| | | | | 12 |
| 0.020267367 | ENSG00000105223 | Q8IV08 | PLD3 | phospholipase D family member |
| | | | | 3 |
| 0.020267367 | ENSG00000197019 | Q9UHV2 | SERTAD1 | SERTA domain containing 1 |
| 0.020618906 | ENSG00000168092 | P68402 | PAFAH1B2 | platelet activating factor |
| | | | | acetylhydrolase 1b catalytic |
| | | | | subunit 2 |
| 0.020658257 | ENSG00000147044 | O14936 | CASK | calcium/calmodulin dependent |
| | | | | serine protein kinase |
| 0.020733242 | ENSG00000079332 | Q9NR31 | SAR1A | secretion associated Ras related |
| | | | | GTPase 1A |
| 0.020779351 | ENSG00000139644 | P55061 | TMBIM6 | transmembrane BAX inhibitor |
| | | | | motif containing 6 |
| 0.02088332 | ENSG00000101856 | O00264 | PGRMC1 | progesterone receptor |
| | | | | membrane component 1 |
| 0.020889692 | ENSG00000177426 | Q15583 | TGIF1 | TGFB induced factor homeobox |
| | | | | 1 |
| 0.020915028 | ENSG00000103051 | Q9H9E3 | COG4 | component of oligomeric golgi |
| | | | | complex 4 |
| 0.021014086 | ENSG00000065559 | P45985 | MAP2K4 | mitogen-activated protein |
| | | | | kinase kinase 4 |
| 0.021014086 | ENSG00000149547 | O14681 | EI24 | EI24, autophagy associated |
| | | | | transmembrane protein |
| 0.021066102 | ENSG00000125995 | P60602 | ROMO1 | reactive oxygen species |
| | | | | modulator 1 |
| 0.021066102 | ENSG00000144357 | Q6ZT12 | UBR3 | ubiquitin protein ligase E3 |
| | | | | component n-recognin 3 |
| | | | | (putative) |
| 0.021141477 | ENSG00000103226 | P69849 | NOMO3 | NODAL modulator 3 |
| 0.021317476 | ENSG00000108671 | O00231 | PSMD11 | proteasome 26S subunit, non- |
| | | | | ATPase 11 |
| 0.021411311 | ENSG00000104936 | Q09013 | DMPK | dystrophia myotonica protein |
| | | | | kinase |
| 0.02154543 | ENSG00000160007 | Q9NRY4 | ARHGAP35 | Rho GTPase activating protein |
| | | | | 35 |
| 0.021620689 | ENSG00000142892 | Q92643 | PIGK | phosphatidylinositol glycan |
| | | | | anchor biosynthesis class K |
| 0.021684647 | ENSG00000084073 | O75844 | ZMPSTE24 | zinc metallopeptidase STE24 |
| 0.021705038 | ENSG00000160691 | P29353 | SHC1 | SHC adaptor protein 1 |
| 0.021705038 | ENSG00000177963 | Q9NPQ8 | RIC8A | RIC8 guanine nucleotide |
| | | | | exchange factor A |
| 0.021939401 | ENSG00000196792 | Q13033 | STRN3 | striatin 3 |
| 0.021982145 | ENSG00000160014 | P62158 | CALM3 | calmodulin 3 |
| 0.022011224 | ENSG00000143418 | Q96G23 | CERS2 | ceramide synthase 2 |
| 0.022146407 | ENSG00000012174 | O43462 | MBTPS2 | membrane bound transcription |
| | | | | factor peptidase, site 2 |
| 0.022163442 | ENSG00000181704 | Q96EC8 | YIPF6 | Yip1 domain family member 6 |
| 0.022276529 | ENSG00000181061 | Q9Y241 | HIGD1A | HIG1 hypoxia inducible domain |
| | | | | family member 1A |
| 0.022301164 | ENSG00000169359 | O00400 | SLC33A1 | solute carrier family 33 member |
| | | | | 1 |
| 0.022328325 | ENSG00000006125 | P63010 | AP2B1 | adaptor related protein complex |
| | | | | 2 beta 1 subunit |
| 0.022328325 | ENSG00000134852 | O15516 | CLOCK | clock circadian regulator |
| 0.022440957 | ENSG00000174915 | Q9BVG9 | PTDSS2 | phosphatidylserine synthase 2 |
| 0.022501117 | ENSG00000173744 | P52594 | AGFG1 | ArfGAP with FG repeats 1 |
| 0.022501117 | ENSG00000188419 | P24386 | CHM | CHM, Rab escort protein 1 |
| 0.022501117 | ENSG00000278053 | Q9Y2R4 | DDX52 | DEAD-box helicase 52 |
| 0.022504634 | ENSG00000104419 | Q92597 | NDRG1 | N-myc downstream regulated 1 |
| 0.022704105 | ENSG00000254685 | O14772 | FPGT | fucose-1-phosphate |
| | | | | guanylyltransferase |
| 0.02272195 | ENSG00000141503 | Q8N4C8 | MINK1 | misshapen like kinase 1 |
| 0.02278321 | ENSG00000197879 | O00159 | MYO1C | myosin IC |
| 0.022805083 | ENSG00000102580 | Q13217 | DNAJC3 | DnaJ heat shock protein family |
| | | | | (Hsp40) member C3 |
| 0.022843039 | ENSG00000027847 | Q9UBV7 | B4GALT7 | beta-1,4-galactosyltransferase 7 |
| 0.022939475 | ENSG00000142089 | Q01628 | IFITM3 | interferon induced |
| | | | | transmembrane protein 3 |
| 0.022971826 | ENSG00000048140 | Q96FV3 | TSPAN17 | tetraspanin 17 |
| 0.022975798 | ENSG00000105254 | Q99426 | TBCB | tubulin folding cofactor B |
| 0.023023778 | ENSG00000002834 | Q14847 | LASP1 | LIM and SH3 protein 1 |
| 0.023035822 | ENSG00000108010 | O76003 | GLRX3 | glutaredoxin 3 |
| 0.023273254 | ENSG00000107897 | Q5T8D3 | ACBD5 | acyl-CoA binding domain |
| | | | | containing 5 |
| 0.023374403 | ENSG00000132256 | Q9C035 | TRIM5 | tripartite motif containing 5 |
| 0.023374403 | ENSG00000137710 | P35241 | RDX | radixin |
| 0.023374403 | ENSG00000167397 | Q9BQB6 | VKORC1 | vitamin K epoxide reductase |
| | | | | complex subunit 1 |
| 0.023374403 | ENSG00000173548 | Q8WV41 | SNX33 | sorting nexin 33 |
| 0.023475887 | ENSG00000068001 | Q12891 | HYAL2 | hyaluronoglucosaminidase 2 |
| 0.02348807 | ENSG00000138190 | Q8TAG9 | EXOC6 | exocyst complex component 6 |
| 0.023681009 | ENSG00000120694 | Q92598 | HSPH1 | heat shock protein family H |
| | | | | (Hsp110) member 1 |
| 0.023819647 | ENSG00000108784 | P54802 | NAGLU | N-acetyl-alpha-glucosaminidase |
| 0.023984723 | ENSG00000196235 | O00267 | SUPT5H | SPT5 homolog, DSIF elongation |
| | | | | factor subunit |
| 0.023995219 | ENSG00000158019 | Q9NXR7 | BRE | brain and reproductive organ- |
| | | | | expressed (TNFRSF1A |
| | | | | modulator) |
| 0.023995219 | ENSG00000182831 | Q14CZ0 | C16orf72 | chromosome 16 open reading |
| | | | | frame 72 |
| 0.024271888 | ENSG00000081087 | Q86WC4 | OSTM1 | osteopetrosis associated |
| | | | | transmembrane protein 1 |
| 0.024334256 | ENSG00000140564 | P09958 | FURIN | furin, paired basic amino acid |
| | | | | cleaving enzyme |
| 0.024561899 | ENSG00000145817 | Q969M3 | YIPF5 | Yip1 domain family member 5 |
| 0.024736195 | ENSG00000140612 | P67812 | SEC11A | SEC11 homolog A, signal |
| | | | | peptidase complex subunit |
| 0.024736195 | ENSG00000144455 | Q8NBK3 | SUMF1 | sulfatase modifying factor 1 |
| 0.024740308 | ENSG00000067225 | P14618 | PKM | pyruvate kinase, muscle |
| 0.024740308 | ENSG00000082996 | O43567 | RNF13 | ring finger protein 13 |
| 0.024740308 | ENSG00000100926 | O15321 | TM9SF1 | transmembrane 9 superfamily |
| | | | | member 1 |
| 0.024740308 | ENSG00000113068 | O60925 | PFDN1 | prefoldin subunit 1 |
| 0.024740308 | ENSG00000136205 | Q68CZ2 | TNS3 | tensin 3 |
| 0.024740308 | ENSG00000138835 | P49796 | RGS3 | regulator of G-protein signaling |
| | | | | 3 |
| 0.024740308 | ENSG00000214530 | Q9Y365 | STARD10 | StAR related lipid transfer |
| | | | | domain containing 10 |
| 0.02474102 | ENSG00000189241 | Q9H0U9 | TSPYL1 | TSPY like 1 |
| 0.02483538 | ENSG00000146409 | Q6NT16 | SLC18B1 | solute carrier family 18 member |
| | | | | B1 |
| 0.025132095 | ENSG00000068323 | P19532 | TFE3 | transcription factor binding to |
| | | | | IGHM enhancer 3 |
| 0.025321955 | ENSG00000196914 | Q9NZN5 | ARHGEF12 | Rho guanine nucleotide |
| | | | | exchange factor 12 |
| 0.025373219 | ENSG00000158604 | Q7Z7H5 | TMED4 | transmembrane p24 trafficking |
| | | | | protein 4 |
| 0.02541766 | ENSG00000136044 | Q8NEU8 | APPL2 | adaptor protein, |
| | | | | phosphotyrosine interacting |
| | | | | with PH domain and leucine |
| | | | | zipper 2 |
| 0.025423865 | ENSG00000197226 | Q66K14 | TBC1D9B | TBC1 domain family member 9B |
| 0.025550872 | ENSG00000100442 | Q00688 | FKBP3 | FK506 binding protein 3 |
| 0.02562673 | ENSG00000086062 | P15291 | B4GALT1 | beta-1,4-galactosyltransferase 1 |
| 0.02562673 | ENSG00000120549 | Q5T5P2 | KIAA1217 | KIAA1217 |
| 0.02562673 | ENSG00000188997 | Q4G0X4 | KCTD21 | potassium channel |
| | | | | tetramerization domain |
| | | | | containing 21 |
| 0.025890043 | ENSG00000130589 | Q9BYK8 | HELZ2 | helicase with zinc finger 2 |
| 0.026157751 | ENSG00000109079 | Q13829 | TNFAIP1 | TNF alpha induced protein 1 |
| 0.026194339 | ENSG00000205155 | Q9NZ42 | PSENEN | presenilin enhancer gamma- |
| | | | | secretase subunit |
| 0.026319329 | ENSG00000167770 | Q96FW1 | OTUB1 | OTU deubiquitinase, ubiquitin |
| | | | | aldehyde binding 1 |
| 0.026355621 | ENSG00000198585 | Q96DE0 | NUDT16 | nudix hydrolase 16 |
| 0.026796801 | ENSG00000143013 | P61968 | LMO4 | LIM domain only 4 |
| 0.026796801 | ENSG00000160055 | Q8WY98 | TMEM234 | transmembrane protein 234 |
| 0.026820474 | ENSG00000169857 | Q9NQS1 | AVEN | apoptosis and caspase |
| | | | | activation inhibitor |
| 0.027204367 | ENSG00000142192 | P05067 | APP | amyloid beta precursor protein |
| 0.027275338 | ENSG00000063245 | Q9Y6I3 | EPN1 | epsin 1 |
| 0.027275338 | ENSG00000204386 | Q99519 | NEU1 | neuraminidase 1 |
| 0.027292509 | ENSG00000121289 | Q96ST8 | CEP89 | centrosomal protein 89 |
| 0.027339092 | ENSG00000115540 | Q9Y3A3 | MOB4 | MOB family member 4, phocein |
| 0.027339183 | ENSG00000180573 | Q93077 | HIST1H2AC | histone cluster 1, H2ac |
| 0.027535195 | ENSG00000163482 | Q9NRP7 | STK36 | serine/threonine kinase 36 |
| 0.027594473 | ENSG00000044574 | P11021 | HSPA5 | heat shock protein family A |
| | | | | (Hsp70) member 5 |
| 0.027654179 | ENSG00000154813 | Q96FX2 | DPH3 | diphthamide biosynthesis 3 |
| 0.02787703 | ENSG00000095139 | P48444 | ARCN1 | archain 1 |
| 0.027887945 | ENSG00000196878 | Q13751 | LAMB3 | laminin subunit beta 3 |
| 0.027974749 | ENSG00000135677 | P15586 | GNS | glucosamine (N-acetyl)-6- |
| | | | | sulfatase |
| 0.027974749 | ENSG00000182544 | Q6N075 | MFSD5 | major facilitator superfamily |
| | | | | domain containing 5 |
| 0.02804485 | ENSG00000027697 | P15260 | IFNGR1 | interferon gamma receptor 1 |
| 0.028360308 | ENSG00000151773 | Q5T0U0 | CCDC122 | coiled-coil domain containing |
| | | | | 122 |
| 0.028461118 | ENSG00000169905 | Q8NFQ8 | TOR1AIP2 | torsin 1A interacting protein 2 |
| 0.028465391 | ENSG00000277258 | P35227 | PCGF2 | polycomb group ring finger 2 |
| 0.028583416 | ENSG00000128590 | Q9UBS3 | DNAJB9 | DnaJ heat shock protein family |
| | | | | (Hsp40) member B9 |
| 0.028645389 | ENSG00000197959 | Q9UQ16 | DNM3 | dynamin 3 |
| 0.028712644 | ENSG00000115946 | Q9NRX1 | PNO1 | partner of NOB1 homolog |
| 0.028712644 | ENSG00000155660 | P13667 | PDIA4 | protein disulfide isomerase |
| | | | | family A member 4 |
| 0.028838079 | ENSG00000117308 | Q14376 | GALE | UDP-galactose-4-epimerase |
| 0.028910401 | ENSG00000006712 | Q8N7H5 | PAF1 | PAF1 homolog, Paf1/RNA |
| | | | | polymerase II complex |
| | | | | component |
| 0.029062905 | ENSG00000140365 | Q9H0A8 | COMMD4 | COMM domain containing 4 |
| 0.029176471 | ENSG00000054523 | O60333 | KIF1B | kinesin family member 1B |
| 0.029343242 | ENSG00000172216 | P17676 | CEBPB | CCAAT/enhancer binding |
| | | | | protein beta |
| 0.029446921 | ENSG00000164040 | O15173 | PGRMC2 | progesterone receptor |
| | | | | membrane component 2 |
| 0.029452197 | ENSG00000162736 | Q92542 | NCSTN | nicastrin |
| 0.029460822 | ENSG00000169446 | Q8N4V1 | MMGT1 | membrane magnesium |
| | | | | transporter 1 |
| 0.029526539 | ENSG00000145743 | Q9UF56 | FBXL17 | F-box and leucine rich repeat |
| | | | | protein 17 |
| 0.029526539 | ENSG00000168710 | O43865 | AHCYL1 | adenosylhomocysteinase like 1 |
| 0.029802673 | ENSG00000123178 | Q5W111 | SPRYD7 | SPRY domain containing 7 |
| 0.029917122 | ENSG00000197774 | A4GXA9 | EME2 | essential meiotic structure- |
| | | | | specific endonuclease subunit 2 |
| 0.030113663 | ENSG00000116005 | Q9UHG3 | PCYOX1 | prenylcysteine oxidase 1 |
| 0.03036926 | ENSG00000241399 | Q8IX05 | CD302 | CD302 molecule |
| 0.03056315 | ENSG00000087074 | O75807 | PPP1R15A | protein phosphatase 1 |
| | | | | regulatory subunit 15A |
| 0.030644082 | ENSG00000141232 | P50616 | TOB1 | transducer of ERBB2, 1 |
| 0.03070953 | ENSG00000042753 | P53680 | AP2S1 | adaptor related protein complex |
| | | | | 2 sigma 1 subunit |
| 0.03070953 | ENSG00000166228 | P61457 | PCBD1 | pterin-4 alpha-carbinolamine |
| | | | | dehydratase 1 |
| 0.030732601 | ENSG00000125257 | O15439 | ABCC4 | ATP binding cassette subfamily |
| | | | | C member 4 |
| 0.030735674 | ENSG00000164172 | O96007 | MOCS2 | molybdenum cofactor synthesis |
| | | | | 2 |
| 0.030735674 | ENSG00000164172 | O96033 | MOCS2 | molybdenum cofactor synthesis |
| | | | | 2 |
| 0.030754139 | ENSG00000135956 | O75204 | TMEM127 | transmembrane protein 127 |
| 0.030870846 | ENSG00000115084 | Q8WV83 | SLC35F5 | solute carrier family 35 member |
| | | | | F5 |
| 0.030904681 | ENSG00000109919 | Q9Y6C9 | MTCH2 | mitochondrial carrier 2 |
| 0.031106894 | ENSG00000162542 | Q5TGY1 | TMCO4 | transmembrane and coiled-coil |
| | | | | domains 4 |
| 0.031219427 | ENSG00000180304 | O95190 | OAZ2 | ornithine decarboxylase |
| | | | | antizyme 2 |
| 0.031431772 | ENSG00000132640 | Q9Y2F9 | BTBD3 | BTB domain containing 3 |
| 0.031431772 | ENSG00000164715 | Q8IWU2 | LMTK2 | lemur tyrosine kinase 2 |
| 0.031761418 | ENSG00000006282 | Q8TB22 | SPATA20 | spermatogenesis associated 20 |
| 0.031796472 | ENSG00000051009 | Q8N612 | FAM160A2 | family with sequence similarity |
| | | | | 160 member A2 |
| 0.031796472 | ENSG00000186073 | Q9Y2V0 | C15orf41 | chromosome 15 open reading |
| | | | | frame 41 |
| 0.031899451 | ENSG00000121578 | O60513 | B4GALT4 | beta-1,4-galactosyltransferase 4 |
| 0.031978416 | ENSG00000040531 | O60931 | CTNS | cystinosin, lysosomal cystine |
| | | | | transporter |
| 0.032035566 | ENSG00000103024 | Q13232 | NME3 | NME/NM23 nucleoside |
| | | | | diphosphate kinase 3 |
| 0.032068174 | ENSG00000077713 | Q8WUT9 | SLC25A43 | solute carrier family 25 member |
| | | | | 43 |
| 0.03210143 | ENSG00000176853 | Q658Y4 | FAM91A1 | family with sequence similarity |
| | | | | 91 member A1 |
| 0.032277485 | ENSG00000101346 | Q9H488 | POFUT1 | protein O-fucosyltransferase 1 |
| 0.03233454 | ENSG00000154309 | Q96F81 | DISP1 | dispatched RND transporter |
| | | | | family member 1 |
| 0.032420605 | ENSG00000117906 | Q14257 | RCN2 | reticulocalbin 2 |
| 0.032457594 | ENSG00000170315 | P0CG47 | UBB | ubiquitin B |
| 0.032514165 | ENSG00000126461 | Q9H7N4 | SCAF1 | SR-related CTD associated factor |
| | | | | 1 |
| 0.032526952 | ENSG00000126562 | Q96J92 | WNK4 | WNK lysine deficient protein |
| | | | | kinase 4 |
| 0.032630245 | ENSG00000126790 | Q96EM0 | L3HYPDH | trans-L-3-hydroxyproline |
| | | | | dehydratase |
| 0.032680082 | ENSG00000109586 | Q86SF2 | GALNT7 | polypeptide N- |
| | | | | acetylgalactosaminyltransferase |
| | | | | 7 |
| 0.032721317 | ENSG00000113658 | Q99717 | SMAD5 | SMAD family member 5 |
| 0.032721317 | ENSG00000130725 | P61081 | UBE2M | ubiquitin conjugating enzyme E2 |
| | | | | M |
| 0.032820621 | ENSG00000183696 | Q16831 | UPP1 | uridine phosphorylase 1 |
| 0.032872158 | ENSG00000167900 | P04183 | TK1 | thymidine kinase 1 |
| 0.032885008 | ENSG00000147164 | Q9UMY4 | SNX12 | sorting nexin 12 |
| 0.032948084 | ENSG00000198642 | Q9P2J3 | KLHL9 | kelch like family member 9 |
| 0.032951754 | ENSG00000071553 | Q15904 | ATP6AP1 | ATPase H+ transporting |
| | | | | accessory protein 1 |
| 0.032956944 | ENSG00000143624 | Q68E01 | INTS3 | integrator complex subunit 3 |
| 0.033050509 | ENSG00000101079 | Q9UGV2 | NDRG3 | NDRG family member 3 |
| 0.033386231 | ENSG00000107862 | Q92538 | GBF1 | golgi brefeldin A resistant |
| | | | | guanine nucleotide exchange |
| | | | | factor 1 |
| 0.033427725 | ENSG00000125733 | Q15642 | TRIP10 | thyroid hormone receptor |
| | | | | interactor 10 |
| 0.03345748 | ENSG00000144061 | O15259 | NPHP1 | nephrocystin 1 |
| 0.033619281 | ENSG00000276023 | O95147 | DUSP14 | dual specificity phosphatase 14 |
| 0.033848755 | ENSG00000151208 | Q8TDM6 | DLG5 | discs large MAGUK scaffold |
| | | | | protein 5 |
| 0.033881213 | ENSG00000204619 | O60927 | PPP1R11 | protein phosphatase 1 |
| | | | | regulatory inhibitor subunit 11 |
| 0.034032818 | ENSG00000110429 | Q9UK99 | FBXO3 | F-box protein 3 |
| 0.034444378 | ENSG00000170522 | Q9H5J4 | ELOVL6 | ELOVL fatty acid elongase 6 |
| 0.034569212 | ENSG00000181104 | P25116 | F2R | coagulation factor II thrombin |
| | | | | receptor |
| 0.034877798 | ENSG00000188313 | O15162 | PLSCR1 | phospholipid scramblase 1 |
| 0.034990429 | ENSG00000137996 | O00442 | RTCA | RNA 3′-terminal phosphate |
| | | | | cyclase |
| 0.03499554 | ENSG00000181991 | P82912 | MRPS11 | mitochondrial ribosomal protein |
| | | | | S11 |
| 0.03501878 | ENSG00000065243 | Q16513 | PKN2 | protein kinase N2 |
| 0.035124321 | ENSG00000100644 | Q16665 | HIF1A | hypoxia inducible factor 1 alpha |
| | | | | subunit |
| 0.035315498 | ENSG00000068137 | Q7Z736 | PLEKHH3 | pleckstrin homology, MyTH4 |
| | | | | and FERM domain containing H3 |
| 0.035422037 | ENSG00000157379 | Q96LJ7 | DHRS1 | dehydrogenase/reductase 1 |
| 0.035422037 | ENSG00000179119 | Q68D10 | SPTY2D1 | SPT2 chromatin protein domain |
| | | | | containing 1 |
| 0.035486703 | ENSG00000239305 | O00237 | RNF103 | ring finger protein 103 |
| 0.035590638 | ENSG00000125037 | Q9P0I2 | EMC3 | ER membrane protein complex |
| | | | | subunit 3 |
| 0.036023076 | ENSG00000086065 | Q9NZZ3 | CHMP5 | charged multivesicular body |
| | | | | protein 5 |
| 0.036332153 | ENSG00000185515 | P46736 | BRCC3 | BRCA1/BRCA2-containing |
| | | | | complex subunit 3 |
| 0.036355137 | ENSG00000197780 | Q15543 | TAF13 | TATA-box binding protein |
| | | | | associated factor 13 |
| 0.036856298 | ENSG00000101160 | Q9UBR2 | CTSZ | cathepsin Z |
| 0.037231687 | ENSG00000174695 | Q8TBQ9 | TMEM167A | transmembrane protein 167A |
| 0.03728095 | ENSG00000102265 | P01033 | TIMP1 | TIMP metallopeptidase inhibitor |
| | | | | 1 |
| 0.037526353 | ENSG00000134107 | O14503 | BHLHE40 | basic helix-loop-helix family |
| | | | | member e40 |
| 0.037531926 | ENSG00000123989 | Q8IZ52 | CHPF | chondroitin polymerizing factor |
| 0.037531926 | ENSG00000135535 | Q04900 | CD164 | CD164 molecule |
| 0.037531926 | ENSG00000147459 | Q9H7D0 | DOCK5 | dedicator of cytokinesis 5 |
| 0.037531926 | ENSG00000153936 | Q7LGA3 | HS2ST1 | heparan sulfate 2-O- |
| | | | | sulfotransferase 1 |
| 0.037549303 | ENSG00000143952 | Q9P1Q0 | VPS54 | VPS54, GARP complex subunit |
| 0.037584723 | ENSG00000164574 | Q86SR1 | GALNT10 | polypeptide N- |
| | | | | acetylgalactosaminyltransferase |
| | | | | 10 |
| 0.037624845 | ENSG00000136152 | Q96JB2 | COG3 | component of oligomeric golgi |
| | | | | complex 3 |
| 0.037693794 | ENSG00000159720 | P61421 | ATP6V0D1 | ATPase H+ transporting V0 |
| | | | | subunit d1 |
| 0.037704811 | ENSG00000040633 | Q9BUL5 | PHF23 | PHD finger protein 23 |
| 0.037821408 | ENSG00000184076 | Q9UDW1 | UQCR10 | ubiquinol-cytochrome c |
| | | | | reductase, complex III subunit X |
| 0.03787287 | ENSG00000163468 | P49368 | CCT3 | chaperonin containing TCP1 |
| | | | | subunit 3 |
| 0.037921726 | ENSG00000147955 | Q99720 | SIGMAR1 | sigma non-opioid intracellular |
| | | | | receptor 1 |
| 0.037924967 | ENSG00000103429 | Q9NZS9 | BFAR | bifunctional apoptosis regulator |
| 0.038180711 | ENSG00000103591 | Q6PD74 | AAGAB | alpha- and gamma-adaptin |
| | | | | binding protein |
| 0.038219614 | ENSG00000138600 | Q8TCT8 | SPPL2A | signal peptide peptidase like 2A |
| 0.038661472 | ENSG00000145022 | P57738 | TCTA | T-cell leukemia translocation |
| | | | | altered |
| 0.038752512 | ENSG00000105404 | Q9UI14 | RABAC1 | Rab acceptor 1 |
| 0.038788791 | ENSG00000067248 | Q7Z478 | DHX29 | DEAH-box helicase 29 |
| 0.03879065 | ENSG00000092931 | O43934 | MFSD11 | major facilitator superfamily |
| | | | | domain containing 11 |
| 0.038790695 | ENSG00000181038 | Q86XA0 | METTL23 | methyltransferase like 23 |
| 0.038831218 | ENSG00000135269 | Q9UGI8 | TES | testin LIM domain protein |
| 0.03888443 | ENSG00000177042 | Q96HE8 | TMEM80 | transmembrane protein 80 |
| 0.038964227 | ENSG00000102100 | P78381 | SLC35A2 | solute carrier family 35 member |
| | | | | A2 |
| 0.039119537 | ENSG00000100554 | Q9Y5K8 | ATP6V1D | ATPase H+ transporting V1 |
| | | | | subunit D |
| 0.039231471 | ENSG00000144566 | P20339 | RAB5A | RAB5A, member RAS oncogene |
| | | | | family |
| 0.039257576 | ENSG00000188229 | P68371 | TUBB4B | tubulin beta 4B class IVb |
| 0.039330825 | ENSG00000134153 | Q9NPA0 | EMC7 | ER membrane protein complex |
| | | | | subunit 7 |
| 0.03935228 | ENSG00000113384 | Q9H4A6 | GOLPH3 | golgi phosphoprotein 3 |
| 0.03967354 | ENSG00000072134 | O95208 | EPN2 | epsin 2 |
| 0.03967354 | ENSG00000169991 | Q5TF58 | IFFO2 | intermediate filament family |
| | | | | orphan 2 |
| 0.039833669 | ENSG00000165424 | Q8N2G6 | ZCCHC24 | zinc finger CCHC-type containing |
| | | | | 24 |
| 0.040061872 | ENSG00000170899 | O15217 | GSTA4 | glutathione S-transferase alpha |
| | | | | 4 |
| 0.040213406 | ENSG00000005020 | O75563 | SKAP2 | src kinase associated |
| | | | | phosphoprotein 2 |
| 0.040213406 | ENSG00000170876 | Q9BTV4 | TMEM43 | transmembrane protein 43 |
| 0.040213406 | ENSG00000185650 | Q07352 | ZFP36L1 | ZFP36 ring finger protein like 1 |
| 0.040297288 | ENSG00000113719 | Q969X5 | ERGIC1 | endoplasmic reticulum-golgi |
| | | | | intermediate compartment 1 |
| 0.040517425 | ENSG00000064601 | P10619 | CTSA | cathepsin A |
| 0.040906257 | ENSG00000173230 | Q14789 | GOLGB1 | golgin B1 |
| 0.041111735 | ENSG00000123353 | Q53FV1 | ORMDL2 | ORMDL sphingolipid |
| | | | | biosynthesis regulator 2 |
| 0.041453962 | ENSG00000138463 | Q96SL1 | DIRC2 | disrupted in renal carcinoma 2 |
| 0.041462984 | ENSG00000188010 | Q502X0 | MORN2 | MORN repeat containing 2 |
| 0.041498042 | ENSG00000137364 | P51580 | TPMT | thiopurine S-methyltransferase |
| 0.041632148 | ENSG00000033011 | Q9BT22 | ALG1 | ALG1, |
| | | | | chitobiosyldiphosphodolichol |
| | | | | beta-mannosyltransferase |
| 0.041703935 | ENSG00000173486 | P26885 | FKBP2 | FK506 binding protein 2 |
| 0.041806889 | ENSG00000204843 | Q14203 | DCTN1 | dynactin subunit 1 |
| 0.04200599 | ENSG00000184281 | Q9Y5U2 | TSSC4 | tumor suppressing |
| | | | | subtransferable candidate 4 |
| 0.042064287 | ENSG00000004864 | Q9UJS0 | SLC25A13 | solute carrier family 25 member |
| | | | | 13 |
| 0.042179458 | ENSG00000005889 | P17010 | ZFX | zinc finger protein, X-linked |
| 0.042179458 | ENSG00000159335 | P20962 | PTMS | parathymosin |
| 0.04220684 | ENSG00000203879 | P31150 | GDI1 | GDP dissociation inhibitor 1 |
| 0.042389783 | ENSG00000100211 | Q9Y3M2 | CBY1 | chibby family member 1, beta |
| | | | | catenin antagonist |
| 0.042451011 | ENSG00000089063 | Q96A57 | TMEM230 | transmembrane protein 230 |
| 0.042451011 | ENSG00000164902 | Q9H814 | PHAX | phosphorylated adaptor for RNA |
| | | | | export |
| 0.042452867 | ENSG00000132825 | O95685 | PPP1R3D | protein phosphatase 1 |
| | | | | regulatory subunit 3D |
| 0.042463923 | ENSG00000118363 | Q15005 | SPCS2 | signal peptidase complex |
| | | | | subunit 2 |
| 0.042590387 | ENSG00000102309 | Q9Y237 | PIN4 | peptidylprolyl cis/trans |
| | | | | isomerase, NIMA-interacting 4 |
| 0.042675505 | ENSG00000166971 | Q9H8T0 | AKTIP | AKT interacting protein |
| 0.042778265 | ENSG00000119927 | Q9HCL2 | GPAM | glycerol-3-phosphate |
| | | | | acyltransferase, mitochondrial |
| 0.042778265 | ENSG00000145012 | Q93052 | LPP | LIM domain containing |
| | | | | preferred translocation partner |
| | | | | in lipoma |
| 0.043076002 | ENSG00000164828 | O94901 | SUN1 | Sad1 and UNC84 domain |
| | | | | containing 1 |
| 0.043076002 | ENSG00000165813 | Q7Z3E2 | CCDC186 | coiled-coil domain containing |
| | | | | 186 |
| 0.043212454 | ENSG00000182768 | Q9NPE2 | NGRN | neugrin, neurite outgrowth |
| | | | | associated |
| 0.043329129 | ENSG00000174684 | O43505 | B4GAT1 | beta-1,4-glucuronyltransferase |
| | | | | 1 |
| 0.043472838 | ENSG00000116791 | Q08257 | CRYZ | crystallin zeta |
| 0.043901312 | ENSG00000080845 | Q9Y2H0 | DLGAP4 | DLG associated protein 4 |
| 0.044146407 | ENSG00000166579 | Q9GZM8 | NDEL1 | nudE neurodevelopment |
| | | | | protein 1 like 1 |
| 0.044206677 | ENSG00000253797 | Q5TAP6 | UTP14C | UTP14, small subunit |
| | | | | processome component |
| | | | | homolog C (S. cerevisiae) |
| 0.04420706 | ENSG00000117475 | Q9H2G9 | BLZF1 | basic leucine zipper nuclear |
| | | | | factor 1 |
| 0.044304303 | ENSG00000115514 | O14530 | TXNDC9 | thioredoxin domain containing 9 |
| 0.044304303 | ENSG00000165487 | Q8IYU8 | MICU2 | mitochondrial calcium uptake 2 |
| 0.044304303 | ENSG00000178761 | Q5XKK7 | FAM219B | family with sequence similarity |
| | | | | 219 member B |
| 0.04431922 | ENSG00000186432 | O00629 | KPNA4 | karyopherin subunit alpha 4 |
| 0.044462839 | ENSG00000138107 | P61163 | ACTR1A | ARP1 actin-related protein 1 |
| | | | | homolog A, centractin alpha |
| 0.044467397 | ENSG00000204388 | P0DMV9 | HSPA1B | heat shock protein family A |
| | | | | (Hsp70) member 1B |
| 0.044653938 | ENSG00000124155 | Q969N2 | PIGT | phosphatidylinositol glycan |
| | | | | anchor biosynthesis class T |
| 0.044760219 | ENSG00000112697 | Q9NV96 | TMEM30A | transmembrane protein 30A |
| 0.044760219 | ENSG00000123131 | Q13162 | PRDX4 | peroxiredoxin 4 |
| 0.044910264 | ENSG00000082153 | Q7L1Q6 | BZW1 | basic leucine zipper and W2 |
| | | | | domains 1 |
| 0.044925489 | ENSG00000090674 | Q9GZU1 | MCOLN1 | mucolipin 1 |
| 0.045041716 | ENSG00000084234 | Q06481 | APLP2 | amyloid beta precursor like |
| | | | | protein 2 |
| 0.045241496 | ENSG00000218891 | Q8NAF0 | ZNF579 | zinc finger protein 579 |
| 0.045243639 | ENSG00000118985 | O00472 | ELL2 | elongation factor for RNA |
| | | | | polymerase II 2 |
| 0.045256372 | ENSG00000147872 | Q99541 | PLIN2 | perilipin 2 |
| 0.045359327 | ENSG00000171475 | Q8TF74 | WIPF2 | WAS/WASL interacting protein |
| | | | | family member 2 |
| 0.045387449 | ENSG00000157637 | Q9HBR0 | SLC38A10 | solute carrier family 38 member |
| | | | | 10 |
| 0.045425423 | ENSG00000277161 | Q7Z7B1 | PIGW | phosphatidylinositol glycan |
| | | | | anchor biosynthesis class W |
| 0.045499395 | ENSG00000166598 | P14625 | HSP90B1 | heat shock protein 90 beta |
| | | | | family member 1 |
| 0.045535428 | ENSG00000064999 | Q92625 | ANKS1A | ankyrin repeat and sterile alpha |
| | | | | motif domain containing 1A |
| 0.045535428 | ENSG00000110696 | O00193 | C11orf58 | chromosome 11 open reading |
| | | | | frame 58 |
| 0.045626452 | ENSG00000157800 | Q8NCC5 | SLC37A3 | solute carrier family 37 member |
| | | | | 3 |
| 0.045997387 | ENSG00000011426 | Q9NQW6 | ANLN | anillin actin binding protein |
| 0.046015412 | ENSG00000169410 | P43378 | PTPN9 | protein tyrosine phosphatase, |
| | | | | non-receptor type 9 |
| 0.046262113 | ENSG00000182551 | Q9BV57 | ADI1 | acireductone dioxygenase 1 |
| 0.046281879 | ENSG00000102401 | Q9UH62 | ARMCX3 | armadillo repeat containing, X- |
| | | | | linked 3 |
| 0.046519762 | ENSG00000152492 | Q8IVM0 | CCDC50 | coiled-coil domain containing 50 |
| 0.046566127 | ENSG00000173914 | Q9BQ04 | RBM4B | RNA binding motif protein 4B |
| 0.046679259 | ENSG00000184014 | Q6IQ26 | DENND5A | DENN domain containing 5A |
| 0.047141814 | ENSG00000007341 | Q8TDW4 | ST7L | suppression of tumorigenicity 7 |
| | | | | like |
| 0.047279717 | ENSG00000144746 | O75915 | ARL6IP5 | ADP ribosylation factor like |
| | | | | GTPase 6 interacting protein 5 |
| 0.047429441 | ENSG00000038382 | O75962 | TRIO | trio Rho guanine nucleotide |
| | | | | exchange factor |
| 0.047456477 | ENSG00000128595 | O43852 | CALU | calumenin |
| 0.047526749 | ENSG00000138381 | Q9NWL6 | ASNSD1 | asparagine synthetase domain |
| | | | | containing 1 |
| 0.047920833 | ENSG00000132694 | O15085 | ARHGEF11 | Rho guanine nucleotide |
| | | | | exchange factor 11 |
| 0.048094775 | ENSG00000143252 | Q99643 | SDHC | succinate dehydrogenase |
| | | | | complex subunit C |
| 0.048126695 | ENSG00000075399 | Q9Y2B5 | VPS9D1 | VPS9 domain containing 1 |
| 0.048126695 | ENSG00000115694 | O00506 | STK25 | serine/threonine kinase 25 |
| 0.04815672 | ENSG00000088986 | P63167 | DYNLL1 | dynein light chain LC8-type 1 |
| 0.048187536 | ENSG00000168894 | Q9P0P0 | RNF181 | ring finger protein 181 |
| 0.048357242 | ENSG00000165389 | Q969W0 | SPTSSA | serine palmitoyltransferase |
| | | | | small subunit A |
| 0.048376923 | ENSG00000011114 | Q9P203 | BTBD7 | BTB domain containing 7 |
| 0.04838653 | ENSG00000113448 | Q08499 | PDE4D | phosphodiesterase 4D |
| 0.048419944 | ENSG00000157916 | O15258 | RER1 | retention in endoplasmic |
| | | | | reticulum sorting receptor 1 |
| 0.04848521 | ENSG00000099849 | Q02833 | RASSF7 | Ras association domain family |
| | | | | member 7 |
| 0.04848521 | ENSG00000144655 | Q96S65 | CSRNP1 | cysteine and serine rich nuclear |
| | | | | protein 1 |
| 0.048523421 | ENSG00000173905 | O00461 | GOLIM4 | golgi integral membrane protein |
| | | | | 4 |
| 0.04855027 | ENSG00000153551 | Q96FZ5 | CMTM7 | CKLF like MARVEL |
| | | | | transmembrane domain |
| | | | | containing 7 |
| 0.048746809 | ENSG00000117758 | Q86Y82 | STX12 | syntaxin 12 |
| 0.049509579 | ENSG00000176903 | Q8ND90 | PNMA1 | paraneoplastic Ma antigen 1 |
| 0.049600676 | ENSG00000132294 | Q14156 | EFR3A | EFR3 homolog A |
| 0.049647478 | ENSG00000096092 | Q9Y6G1 | TMEM14A | transmembrane protein 14A |
| 0.049675139 | ENSG00000107821 | Q96I82 | KAZALD1 | Kazal type serine peptidase |
| | | | | inhibitor domain 1 |
| 0.049682949 | ENSG00000197045 | P60983 | GMFB | glia maturation factor beta |
| 0.049792447 | ENSG00000105281 | Q15758 | SLC1A5 | solute carrier family 1 member |
| | | | | 5 |
| 0.049852801 | ENSG00000167460 | P67936 | TPM4 | tropomyosin 4 |
| 0.049985173 | ENSG00000119686 | Q9UPI3 | FLVCR2 | feline leukemia virus subgroup C |
| | | | | cellular receptor family member |
| | | | | 2 |

TABLE 3

| # | IPI | | |
|---|---|---|---|
| Peptides | Reference | Gene Symbol | Protein Description |
| 16 | IPI00302592 | FLNA | filamin A, alpha (actin binding |
| | | | protein 280) |
| 14 | IPI00018873 | NAMPT | pre-B-cell colony enhancing factor 1 |
| 13 | IPI00299402 | PC | pyruvate carboxylase |
| 12 | IPI00003865 | HSPA8 | heat shock 70 kDa protein 8 |
| 10 | IPI00219077 | LTA4H | leukotriene A4 hydrolase |
| 10 | IPI00328587 | EDARADD | EDAR-associated death domain |
| 8 | IPI00003935 | HIST2H2BE | histone 2, H2be |
| 8 | IPI00027230 | HSP90B1 | heat shock protein 90 kDa beta |
| | | | (Grp94), member 1 |
| 7 | IPI00010796 | P4HB | procollagen-proline, 2-oxoglutarate |
| | | | 4-dioxygenase (proline 4- |
| | | | hydroxylase), beta polypeptide |
| 7 | IPI00030929 | MYL9 | myosin, light polypeptide 9, |
| | | | regulatory |
| 7 | IPI00215914 | ARF1 | ADP-ribosylation factor 1 |
| 7 | IPI00218319 | TPM3 | tropomyosin 3 |
| 7 | IPI00218570 | PGAM2 | phosphoglycerate mutase 2 |
| | | | (muscle) |
| 7 | IPI00646304 | PPIB | peptidylprolyl isomerase B |
| | | | (cyclophilin B) |
| 6 | IPI00003817 | ARHGDIB | Rho GDP dissociation inhibitor (GDI) |
| | | | beta |
| 6 | IPI00006663 | ALDH2 | aldehyde dehydrogenase 2 family |
| | | | (mitochondrial) |
| 6 | IPI00011134 | HSPA7 | heat shock 70 kDa protein 7 |
| | | | (HSP70B) |
| 6 | IPI00017704 | COTL1 | coactosin-like 1 |
| 6 | IPI00025257 | SEMA7A | semaphorin 7A, GPI membrane |
| | | | anchor (John Milton Hagen blood |
| | | | group) |
| 6 | IPI00292858 | TYMP | endothelial cell growth factor 1 |
| | | | (platelet-derived) |
| 6 | IPI00418471 | VIM | vimentin |
| 6 | IPI00784154 | HSPD1 | 60 kDa heat shock protein, |
| | | | mitochondrial |
| 6 | IPI00909568 | — | |
| 5 | IPI00008274 | CAP1 | CAP, adenylate cyclase-associated |
| | | | protein 1 (yeast) |
| 5 | IPI00027444 | SERPINB1 | serpin peptidase inhibitor, clade B |
| | | | (ovalbumin), member 1 |
| 5 | IPI00027497 | GPI | glucose phosphate isomerase |
| 5 | IPI00028064 | CTSG | cathepsin G |
| 5 | IPI00064201 | FRMPD3 | FERM And PDZ Domain Containing |
| | | | 3) |
| 5 | IPI00182427 | C17orf47 | chromosome 17 open reading |
| | | | frame 47 |
| 5 | IPI00218638 | MYO1F | myosin IF |
| 5 | IPI00221088 | RPS9 | ribosomal protein S9 |
| 5 | IPI00289344 | NCOR1 | nuclear receptor co-repressor 1 |
| 5 | IPI00412579 | RPL10A | ribosomal protein L10a |
| 5 | IPI00418169 | ANXA2 | annexin A2 |
| 4 | IPI00006510 | TUBB1 | tubulin, beta 1 |
| 4 | IPI00009802 | VCAN | chondroitin sulfate proteoglycan 2 |
| | | | (versican) |
| 4 | IPI00010270 | RAC2 | ras-related C3 botulinum toxin |
| | | | substrate 2 (rho family, small GTP |
| | | | binding protein Rac2) |
| 4 | IPI00011253 | RPS3 | ribosomal protein S3 |
| 4 | IPI00012011 | CFL1 | cofilin 1 (non-muscle) |
| 4 | IPI00019755 | GSTO1 | glutathione S-transferase omega 1 |
| 4 | IPI00020436 | RAB11B | RAB11B, member RAS oncogene |
| | | | family |
| 4 | IPI00021263 | YWHAZ | tyrosine 3- |
| | | | monooxygenase/tryptophan 5- |
| | | | monooxygenase activation protein, |
| | | | zeta polypeptide |
| 4 | IPI00025447 | EEF1A1 | chemokine (C-C motif) receptor 5 |
| 4 | IPI00031523 | HSP90AA2 | cytosolic HSP90 protein |
| 4 | IPI00166612 | CMYA5 | cardiomyopathy associated 5 |
| 4 | IPI00171611 | HIST2H3D; | histone H3/o |
| | | HIST2H3C; | |
| | | HIST2H3A | |
| 4 | IPI00186290 | EEF2 | eukaryotic translation elongation |
| | | | factor 2 |
| 4 | IPI00216691 | PFN1 | profilin 1 |
| 4 | IPI00291175 | VCL | vinculin |
| 4 | IPI00294739 | SAMHD1 | SAM domain and HD domain 1 |
| 4 | IPI00299150 | CTSS | cathepsin S |
| 4 | IPI00303476 | ATP5B | ATP synthase, H+ transporting, |
| | | | mitochondrial F1 complex, beta |
| | | | polypeptide |
| 4 | IPI00304922 | LSMD1 | LSM domain containing 1 |
| 4 | IPI00383071 | TPI1; | Triosephosphate Isomerase 1 |
| | | RCTPI1 | Pseudogene 1 |
| 4 | IPI00411291 | PEX1 | peroxisome biogenesis factor 1 |
| 4 | IPI00411633 | HSP90AB1 | heat shock protein 90 kDa alpha |
| | | | (cytosolic), class B member 1 |
| 4 | IPI00414676 | HSP90AB1 | heat shock protein 90 kDa alpha |
| | | | (cytosolic), class B member 1 |
| 4 | IPI00553241 | PCCA | propionyl Coenzyme A carboxylase, |
| | | | alpha polypeptide |
| 4 | IPI00740961 | INT1 | integrator complex subunit 1 |
| 4 | IPI00741317 | CSNK2A1 | Casein Kinase 2 Alpha 1 |
| 4 | IPI00742682 | TPR | Nucleoprotein TPR |
| 3 | IPI00000105 | MVP | major vault protein |
| 3 | IPI00000230 | TPM1 | tropomyosin 1 (alpha) |
| 3 | IPI00000816 | YWHAE | tyrosine 3- |
| | | | monooxygenase/tryptophan 5- |
| | | | monooxygenase activation protein, |
| | | | epsilon polypeptide |
| 3 | IPI00006690 | EPX | eosinophil peroxidase |
| 3 | IPI00007750 | TUBA4A | tubulin, alpha 1 (testis specific) |
| 3 | IPI00010896 | CLIC1 | chloride intracellular channel 1 |
| 3 | IPI00012283 | SEMA3B | sema domain, immunoglobulin |
| | | | domain (Ig), short basic domain, |
| | | | secreted, (semaphorin) 3B |
| 3 | IPI00013495 | ABCF1 | ATP-binding cassette, sub-family F |
| | | | (GCN20), member 1 |
| 3 | IPI00015713 | CDKAL1 | CDK5 regulatory subunit associated |
| | | | protein 1-like 1 |
| 3 | IPI00018534 | HIST1H2BL | histone 1, H2bl |
| 3 | IPI00018744 | TRADD | TNFRSF1A-associated via death |
| | | | domain |
| 3 | IPI00020984 | CANX | calnexin |
| 3 | IPI00026185 | CAPZB | capping protein (actin filament) |
| | | | muscle Z-line, beta |
| 3 | IPI00027481 | ABCB1 | ATP-binding cassette, sub-family B |
| | | | (MDR/TAP), member 1 |
| 3 | IPI00029744 | SSBP1 | single-stranded DNA binding protein |
| | | | 1 |
| 3 | IPI00032134 | SERPINB8 | serpin peptidase inhibitor, clade B |
| | | | (ovalbumin), member 8 |
| 3 | IPI00102864 | HK2 | hexokinase 2 |
| 3 | IPI00107113 | UTP14A | UTP14, U3 small nucleolar |
| | | | ribonucleoprotein, homolog A |
| | | | (yeast) |
| 3 | IPI00165045 | CACNA1E | calcium channel, voltage- |
| | | | dependent, alpha 1E subunit |
| 3 | IPI00171044 | SLFN11 | likely ortholog of mouse schlafen |
| | | | 8/9 |
| 3 | IPI00177498 | LIMCH1 | hypothetical protein |
| 3 | IPI00179298 | HUWE1 | HECT, UBA and WWE domain |
| | | | containing 1 |
| 3 | IPI00216028 | ASB2 | ankyrin repeat and SOCS box- |
| | | | containing 2 |
| 3 | IPI00218081 | DSEL | chromosome 18 open reading |
| | | | frame 4 |
| 3 | IPI00219153 | RPL22 | ribosomal protein L22 |
| 3 | IPI00220486 | TADA3L | transcriptional adaptor 3 (NGG1 |
| | | | homolog, yeast)-like |
| 3 | IPI00221127 | MYLK2 | myosin light chain kinase 2, skeletal |
| | | | muscle |
| 3 | IPI00247110 | ANKRD62 | Ankyrin Repeat Domain 62 |
| 3 | IPI00291939 | SMC1A | SMC1 structural maintenance of |
| | | | chromosomes 1-like 1 (yeast) |
| 3 | IPI00298994 | TLN1 | talin 1 |
| 3 | IPI00300659 | CDC73 | cell division cycle 73, Paf1/RNA |
| | | | polymerase II complex component, |
| | | | homolog (S. cerevisiae) |
| 3 | IPI00335168 | MYL6; MYL6B | myosin, light polypeptide 6, alkali, |
| | | | smooth muscle and non-muscle |
| 3 | IPI00386354 | TTLL3 | Tubulin Tyrosine Ligase Like 3 |
| 3 | IPI00397526 | MYH10 | myosin, heavy polypeptide 10, non- |
| | | | muscle |
| 3 | IPI00414320 | ANXA11 | annexin A11 |
| 3 | IPI00418735 | C17orf97 | hypothetical gene supported by |
| | | | AK128660 |
| 3 | IPI00472200 | COL4A6 | collagen, type IV, alpha 6 |
| 3 | IPI00479743 | POTEE | POTE Ankyrin Domain Family |
| | | | Member E |
| 3 | IPI00550021 | RPL3 | ribosomal protein L3 |
| 3 | IPI00739082 | LOC100131539 | hypothetical protein LOC100131539 |
| 3 | IPI00782992 | SRRM2 | Serine/arginine repetitive matrix |
| | | | protein 2 |
| 3 | IPI00879202 | — | |
| 3 | IPI00887537 | LOC100131480 | Hypothetical protein |
| | | | LOC1001314840 |
| 2 | IPI00000163 | CTNNAL1 | catenin (cadherin-associated |
| | | | protein), alpha-like 1 |
| 2 | IPI00001654 | PCM1 | pericentriolar material 1 |
| 2 | IPI00002649 | PNN | pinin, desmosome associated |
| | | | protein |
| 2 | IPI00002966 | HSPA4 | heat shock 70 kDa protein 4 |
| 2 | IPI00003807 | ACP2 | acid phosphatase 2, lysosomal |
| 2 | IPI00003965 | USP7 | ubiquitin specific peptidase 7 |
| | | | (herpes virus-associated) |
| 2 | IPI00004237 | PHKA2 | phosphorylase kinase, alpha 2 (liver) |
| 2 | IPI00004299 | NUP210L | nucleoporin 210 kDa-like |
| 2 | IPI00004902 | ETFB | electron-transfer-flavoprotein, beta |
| | | | polypeptide |
| 2 | IPI00005118 | HK3 | hexokinase 3 (white cell) |
| 2 | IPI00005160 | ARPC1B | actin related protein 2/3 complex, |
| | | | subunit 1B, 41 kDa |
| 2 | IPI00005161 | ARPC2 | actin related protein 2/3 complex, |
| | | | subunit 2, 34 kDa |
| 2 | IPI00005162 | ARPC3 | actin related protein 2/3 complex, |
| | | | subunit 3, 21 kDa |
| 2 | IPI00006254 | BZRAP1 | benzodiazapine receptor |
| | | | (peripheral) associated protein 1 |
| 2 | IPI00007765 | HSPA9 | heat shock 70 kDa protein 9B |
| | | | (mortalin-2) |
| 2 | IPI00007834 | ANK2 | ankyrin 2, neuronal |
| 2 | IPI00007840 | ABCG8 | ATP-binding cassette, sub-family G |
| | | | (WHITE), member 8 (sterolin 2) |
| 2 | IPI00007913 | ARHGAP26 | Rho GTPase activating protein 26 |
| 2 | IPI00007921 | NRXN2 | neurexin 2 |
| 2 | IPI00008530 | RPLP0 | ribosomal protein, large, P0 |
| 2 | IPI00008832 | GAS1 | growth arrest-specific 1 |
| 2 | IPI00009505 | SNTB2 | syntrophin, beta 2 (dystrophin- |
| | | | associated protein A1, 59 kDa, basic |
| | | | component 2) |
| 2 | IPI00010180 | CES1 | carboxylesterase 1 |
| | | | (monocyte/macrophage serine |
| | | | esterase 1) |
| 2 | IPI00011290 | SULT1C2 | sulfotransferase family, cytosolic, |
| | | | 1C, member 1 |
| 2 | IPI00011416 | ECH1 | enoyl Coenzyme A hydratase 1, |
| | | | peroxisomal |
| 2 | IPI00011454 | GANAB | glucosidase, alpha; neutral AB |
| 2 | IPI00012510 | EMILIN2 | elastin microfibril interfacer 2 |
| 2 | IPI00012728 | ACSL1 | acyl-CoA synthetase long-chain |
| | | | family member 1 |
| 2 | IPI00012858 | KCNQ2 | potassium voltage-gated channel, |
| | | | KQT-like subfamily, member 2 |
| 2 | IPI00013216 | ORC2L | origin recognition complex, subunit |
| | | | 2-like (yeast) |
| 2 | IPI00013306 | GDPD3 | glycerophosphodiester |
| | | | phosphodiesterase domain |
| | | | containing 3 |
| 2 | IPI00013698 | ASAH1 | N-acylsphingosine amidohydrolase |
| | | | (acid ceramidase) 1 |
| 2 | IPI00014843 | LRRC16A | leucine rich repeat containing 16 |
| 2 | IPI00015525 | MMRN2 | multimerin 2 |
| 2 | IPI00016780 | PRDM2 | PR domain containing 2, with ZNF |
| | | | domain |
| 2 | IPI00016801 | GLUD1 | glutamate dehydrogenase 1 |
| 2 | IPI00016832 | PSMA1 | proteasome (prosome, macropain) |
| | | | subunit, alpha type, 1 |
| 2 | IPI00016862 | GSR | glutathione reductase |
| 2 | IPI00017617 | DDX5 | DEAD (Asp-Glu-Ala-Asp) box |
| | | | polypeptide 5 |
| 2 | IPI00017696 | C1S | complement component 1, s |
| | | | subcomponent |
| 2 | IPI00018931 | VPS35 | vacuolar protein sorting 35 (yeast) |
| 2 | IPI00019090 | COL19A1 | collagen, type XIX, alpha 1 |
| 2 | IPI00020265 | ANKRD20A1 | ankyrin repeat domain 20 family, |
| | | | member A1 |
| 2 | IPI00020985 | EP300 | E1A binding protein p300 |
| 2 | IPI00021327 | GRB2 | growth factor receptor-bound |
| | | | protein 2 |
| 2 | IPI00022881 | CLTCL1 | clathrin, heavy polypeptide-like 1 |
| 2 | IPI00023164 | HCN4 | hyperpolarization activated cyclic |
| | | | nucleotide-gated potassium channel |
| | | | 4 |
| 2 | IPI00023532 | LOC26010 | DNA polymerase-transactivated |
| | | | protein 6 |
| 2 | IPI00023724 | MAB21L1 | mab-21-like 1 (C. elegans) |
| 2 | IPI00026119 | UBA1 | ubiquitin-activating enzyme E1 |
| | | | (A1S9T and BN75 temperature |
| | | | sensitivity complementing) |
| 2 | IPI00026314 | GSN | gelsolin (amyloidosis, Finnish type) |
| 2 | IPI00026466 | NIPBL | Nipped-B homolog (Drosophila) |
| 2 | IPI00027255 | MYL6B | myosin light chain 1 slow a |
| 2 | IPI00028031 | ACADVL | acyl-Coenzyme A dehydrogenase, |
| | | | very long chain |
| 2 | IPI00029012 | EIF3A | eukaryotic translation initiation |
| | | | factor 3, subunit 10 theta, |
| | | | 150/170 kDa |
| 2 | IPI00029107 | WRN | Werner syndrome |
| 2 | IPI00029168 | LPA | lipoprotein, Lp(a) |
| 2 | IPI00029822 | SMARCA4 | SWI/SNF related, matrix associated, |
| | | | actin dependent regulator of |
| | | | chromatin, subfamily a, member 4 |
| 2 | IPI00031282 | KIAA1683 | KIAA1683 |
| 2 | IPI00031421 | TPST2 | tyrosylprotein sulfotransferase 2 |
| 2 | IPI00031522 | HADHA | hydroxyacyl-Coenzyme A |
| | | | dehydrogenase/3-ketoacyl- |
| | | | Coenzyme A thiolase/enoyl- |
| | | | Coenzyme A hydratase (trifunctional |
| | | | protein), alpha subunit |
| 2 | IPI00031666 | RP3-402G11.5 | selenoprotein O |
| 2 | IPI00032162 | FOXM1 | forkhead box M1 |
| 2 | IPI00032325 | CSTA | cystatin A (stefin A) |
| 2 | IPI00032875 | ETFDH | electron-transferring-flavoprotein |
| | | | dehydrogenase |
| 2 | IPI00032901 | SAGE1 | sarcoma antigen 1 |
| 2 | IPI00032988 | TSHZ2 | zinc finger protein 218 |
| 2 | IPI00044353 | MED12L | mediator of RNA polymerase II |
| | | | transcription, subunit 12 homolog |
| | | | (yeast)-like |
| 2 | IPI00044583 | TRERF1 | transcriptional regulating factor 1 |
| 2 | IPI00045914 | SPEN | spen homolog, transcriptional |
| | | | regulator (Drosophila) |
| 2 | IPI00064667 | CNDP1 | carnosine dipeptidase 1 |
| | | | (metallopeptidase M20 family) |
| 2 | IPI00065078 | LOC100134025; | KPL2 protein |
| | | SPEF2 | |
| 2 | IPI00075248 | CALM1; CALM3; | calmodulin 2 (phosphorylase kinase, |
| | | CALM2 | delta) |
| 2 | IPI00102677 | TESK2 | testis-specific kinase 2 |
| 2 | IPI00142539 | BCL7B | B-cell CLL/lymphoma 7B |
| 2 | IPI00148768 | TRIOBP | TRIO and F-actin binding protein |
| 2 | IPI00152007 | ARHGEF17 | Rho guanine nucleotide exchange |
| | | | factor (GEF) 17 |
| 2 | IPI00152849 | CCNB3 | cyclin B3 |
| 2 | IPI00163403 | SHKBP1 | SH3KBP1 binding protein 1 |
| 2 | IPI00165261 | SCFD1 | sec1 family domain containing 1 |
| 2 | IPI00165459 | SETD1B | SET Domain Containing 1B, Histone |
| | | | Lysine Methyltransferase |
| 2 | IPI00165579 | CNDP2 | CNDP dipeptidase 2 |
| | | | (metallopeptidase M20 family) |
| 2 | IPI00167201 | CCDC65 | coiled-coil domain containing 65 |
| 2 | IPI00168529 | C1orf125 | chromosome 1 open reading frame |
| | | | 125 |
| 2 | IPI00169426 | NT5C1B | retinol dehydrogenase 14 (all-trans |
| | | | and 9-cis) |
| 2 | IPI00170706 | TMEM2 | transmembrane protein 2 |
| 2 | IPI00171183 | FBXL13 | F-box and leucine-rich repeat |
| | | | protein 13 |
| 2 | IPI00171230 | ERC1 | RAB6 interacting protein 2 |
| 2 | IPI00173347 | HECW1 | HECT, C2 and WW domain |
| | | | containing E3 ubiquitin protein |
| | | | ligase 1 |
| 2 | IPI00179016 | SETD1A | SET domain containing 1A |
| 2 | IPI00179037 | AMOTL1 | angiomotin like 1 |
| 2 | IPI00179330 | UBC; | ribosomal protein S27a |
| | | RPS27A; | |
| | | UBB | |
| 2 | IPI00180384 | DNAH7 | dynein, axonemal, heavy |
| | | | polypeptide 7 |
| 2 | IPI00180672 | MOCS1 | molybdenum cofactor synthesis 1 |
| 2 | IPI00183018 | MICAL2 | microtubule associated |
| | | | monoxygenase, calponin and LIM |
| | | | domain containing 2 |
| 2 | IPI00183046 | PTPN6 | protein tyrosine phosphatase, non- |
| | | | receptor type 6 |
| 2 | IPI00183118 | MARK3 | MAP/microtubule affinity-regulating |
| | | | kinase 3 |
| 2 | IPI00183572 | DOCK7 | dedicator of cytokinesis 7 |
| 2 | IPI00183968 | TPM3 | tropomyosin 3 |
| 2 | IPI00215719 | RPL18 | ribosomal protein L18 |
| 2 | IPI00215918 | ARF4 | ADP-ribosylation factor 4 |
| 2 | IPI00215920 | ARF6 | ADP-ribosylation factor 6 |
| 2 | IPI00216318 | YWHAB | tyrosine 3- |
| | | | monooxygenase/tryptophan 5- |
| | | | monooxygenase activation protein, |
| | | | beta polypeptide |
| 2 | IPI00216362 | OTOF | otoferlin |
| 2 | IPI00216616 | TNIP1 | TNFAIP3 interacting protein 1 |
| 2 | IPI00217178 | C22orf30 | hypothetical protein MGC50372 |
| 2 | IPI00217465 | HIST1H1C | histone 1, H1c |
| 2 | IPI00217802 | FAM186B | chromosome 12 open reading |
| | | | frame 25 |
| 2 | IPI00218693 | APRT | adenine phosphoribosyltransferase |
| 2 | IPI00219757 | GSTP1 | glutathione S-transferase pi |
| 2 | IPI00220109 | ATRX | alpha thalassemia/mental |
| | | | retardation syndrome X-linked |
| | | | (RAD54 homolog, S. cerevisiae) |
| 2 | IPI00220588 | CPXM1 | carboxypeptidase X (M14 family) |
| 2 | IPI00221091 | RPS15A | ribosomal protein S15a |
| 2 | IPI00221295 | TOP3A | topoisomerase (DNA) III alpha |
| 2 | IPI00289776 | MYCBP2 | MYC binding protein 2 |
| 2 | IPI00290337 | EPS8 | epidermal growth factor receptor |
| | | | pathway substrate 8 |
| 2 | IPI00292326 | SPAG1 | sperm associated antigen 1 |
| 2 | IPI00292914 | ANKIB1 | Ankyrin Repeat And IBR Domain |
| | | | Containing 1 |
| 2 | IPI00295485 | HSPA4L | heat shock 70 kDa protein 4-like |
| 2 | IPI00296545 | TXNDC3 | thioredoxin domain containing 3 |
| | | | (spermatozoa) |
| 2 | IPI00296563 | GUF1 | GUF1 GTPase homolog (S. |
| | | | cerevisiae) |
| 2 | IPI00297188 | BAI2 | brain-specific angiogenesis inhibitor |
| | | | 2 |
| 2 | IPI00297859 | MLL2 | myeloid/lymphoid or mixed-lineage |
| | | | leukemia 2 |
| 2 | IPI00298925 | TAF5 | TAF5 RNA polymerase II, TATA box |
| | | | binding protein (TBP)-associated |
| | | | factor, 100 kDa |
| 2 | IPI00299571 | PDIA6 | protein disulfide isomerase family A, |
| | | | member 6 |
| 2 | IPI00299573 | RPL7A | ribosomal protein L7a |
| 2 | IPI00302829 | RB1 | retinoblastoma 1 (including |
| | | | osteosarcoma) |
| 2 | IPI00304925 | HSPA1A; | heat shock 70 kDa protein 1A |
| | | HSPA1B | |
| 2 | IPI00304967 | CYP26A1 | cytochrome P450, family 26, |
| | | | subfamily A, polypeptide 1 |
| 2 | IPI00305010 | CPPED1 | hypothetical protein FLJ11151 |
| 2 | IPI00306322 | COL4A2 | collagen, type IV, alpha 2 |
| 2 | IPI00306929 | MYO18B | myosin XVIIIB |
| 2 | IPI00307829 | CGNL1 | cingulin-like 1 |
| 2 | IPI00329024 | CCDC67 | coiled-coil domain containing 67 |
| 2 | IPI00329236 | PRKCD | protein kinase C, delta |
| 2 | IPI00329560 | RAPGEF4 | Rap guanine nucleotide exchange |
| | | | factor (GEF) 4 |
| 2 | IPI00329637 | C1orf26 | chromosome 1 open reading frame |
| | | | 26 |
| 2 | IPI00337544 | PDE4DIP | phosphodiesterase 4D interacting |
| | | | protein (myomegalin) |
| 2 | IPI00382872 | DUX4C; | double homeobox, 4 |
| | | LOC441056; | |
| | | DUX4 | |
| 2 | IPI00385267 | SRPR | signal recognition particle receptor |
| | | | (‘docking protein’) |
| 2 | IPI00385321 | ZMYND8 | protein kinase C binding protein 1 |
| 2 | IPI00386453 | CALD1 | caldesmon 1 |
| 2 | IPI00395010 | C5orf42 | hypothetical protein FLJ13231 |
| 2 | IPI00395769 | ATP5C1 | ATP synthase, H+ transporting, |
| | | | mitochondrial F1 complex, gamma |
| | | | polypeptide 1 |
| 2 | IPI00396243 | WDR19 | WD repeat domain 19 |
| 2 | IPI00396267 | SNX13 | sorting nexin 13 |
| 2 | IPI00397930 | ZDBF2 | Zinc Finger DBF-Type Containing 2 |
| 2 | IPI00400922 | PDCD11 | programmed cell death 11 |
| 2 | IPI00401026 | CRYBG3 | Crystallin Beta-Gamma Domain |
| | | | Containing 3 |
| 2 | IPI00413755 | TAF4 | TAF4 RNA polymerase II, TATA box |
| | | | binding protein (TBP)-associated |
| | | | factor, 135 kDa |
| 2 | IPI00414347 | ASB18 | ankyrin repeat and SOCS box- |
| | | | containing 18 |
| 2 | IPI00419237 | LAP3 | leucine aminopeptidase 3 |
| 2 | IPI00419273 | CUL4A | cullin 4A |
| 2 | IPI00425688 | RUSC1 | RUN and SH3 domain containing 1 |
| 2 | IPI00433419 | LOC441242 | hypothetical LOC441242 |
| 2 | IPI00436021 | TTN | titin |
| 2 | IPI00440493 | ATP5A1 | ATP synthase, H+ transporting, |
| | | | mitochondrial F1 complex, alpha |
| | | | subunit 1, cardiac muscle |
| 2 | IPI00444262 | NCL | nucleolin |
| 2 | IPI00465294 | CDC5L | CDC5 cell division cycle 5-like (S. |
| | | | pombe) |
| 2 | IPI00470518 | MAD1L1 | MAD1 mitotic arrest deficient-like 1 |
| | | | (yeast) |
| 2 | IPI00479143 | PCNT | pericentrin (kendrin) |
| 2 | IPI00479877 | ALDH9A1 | aldehyde dehydrogenase 9 family, |
| | | | member A1 |
| 2 | IPI00514201 | MYH6 | myosin, heavy polypeptide 6, |
| | | | cardiac muscle, alpha |
| | | | (cardiomyopathy, hypertrophic 1) |
| 2 | IPI00556231 | LOC644936 | hypothetical protein LOC644936 |
| 2 | IPI00640006 | GDI2 | GDP dissociation inhibitor 2 |
| 2 | IPI00641950 | GNB2L1 | guanine nucleotide binding protein |
| | | | (G protein), beta polypeptide 2-like |
| | | | 1 |
| 2 | IPI00643041 | RANP1; | RAN, member RAS oncogene family |
| | | RAN | |
| 2 | IPI00645206 | PCDH17 | protocadherin 17 |
| 2 | IPI00646151 | ATXN1L | Ataxin 1 Like |
| 2 | IPI00739099 | COL5A2 | Collagen Type V Alpha 2 Chain |
| 2 | IPI00749140 | — | |
| 2 | IPI00783392 | RB1CC1 | RB1-inducible coiled-coil protein 1 |
| 2 | IPI00788247 | KIF26A | Kinesin-like protein KIF26A |
| 2 | IPI00791325 | KIAA0020 | Pumilio homolog 3 |
| 2 | IPI00792427 | RAD51AP2 | RAD51-associated protein 2 |
| 2 | IPI00792431 | LMOD3 | Leiomodin-3 |
| 2 | IPI00793199 | ANXA4 | Annexin A4 |
| 2 | IPI00853133 | DDX60L | Probable ATP-dependent RNA |
| | | | helicase DDX60-like |
| 2 | IPI00871718 | — | |
| 2 | IPI00878929 | — | |
| 2 | IPI00884105 | LAMP1 | Lysosome-associated membrane |
| | | | glycoprotein 1 |
| 2 | IPI00887915 | C12orf55 | Cilia And Flagella Associated 54 |
| 2 | IPI00888563 | LOC643395 | Hypothetical protein LOC643395 |
| 2 | IPI00889113 | RPL10P15 | Ribosomal Protein L10 Pseudogene |
| | | | 15 |
| 2 | IPI00914840 | SPRR1A | Small Proline Rich Protein 1A |
| 2 | IPI00923547 | — | |

### Other Embodiments

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of detecting at least one γδ T cell surface ligand in vitro, the method comprising:
(a) contacting a sample with a soluble human γδ T cell receptor (TCR-γδ) tetramer, wherein the sTCR-γδ produces a detectable signal in response to engagement with a γδ T cell surface ligand;
(b) detecting the measurable signal of the sTCR-γδ tetramer, wherein the detectable signal indicates the presence of the γδ T cell surface ligand in the sample.

2. The method of claim 1, wherein the detectable signal is a fluorescent, chemiluminescent, or absorbance signal.

3. The method of claim 1 or claim 2, wherein the sTCR-γδ tetramer is biotinylated and the detectable signal is streptavidin-PE.

4. The method of claim 3, wherein the staining is detected via flow cytometry.

5. The method of any one of claims 1-4, wherein the sTCR-γδ binds to the γδ T cell surface ligand of a Vδ1 T cell.

6. The method of any one of claims 1-5, wherein the sample comprises primary cells or a tumor cell line.

7. The method of any one of claims 1-5, wherein the sample is from a primary tissue, a tumor, inflamed synovium, or intestinal epithelium.

8. The method of any one of claims 1-7, further comprising identifying the γδ T cell surface ligand.

9. The method of claim 8, wherein the γδ T cell surface ligand is identified using RNA-seq and bioinformatics, mass spectrometry, and/or a transfection-based genetic screen.

10. A human synovial soluble TCR-γδ.

11. The human synovial soluble TCR-γδ of claim 10, formulated as a tetramer.

12. The human synovial soluble TCR-γδ tetramer of claim 11, wherein the tetramer is tetramerized with streptavidin-PE or avidin-conjugated magnetic beads.

13. A single vector comprising a T cell receptor (TCR) γ chain sequence and a TCR δ chain sequence, and further comprising two promoters, a tag, and a binding partner sequence.

14. The single vector of claim 13, wherein the tag is a hexa-His tag.

15. The single vector of claim 13, wherein the binding partner sequence is a biotinylation sequence.

16. The vector of claim 13, wherein the two promoters comprise p10 and polyhedron.

17. A method of making the human synovial soluble TCR-γδ, the method comprising transfecting a cell with the vector of claim 13.

18. An anti-cancer therapeutic composition, comprising.
a unique TCR-γδ ligand, wherein the unique TCR-γδ ligand is a protein or a functional fragment thereof of Table 1 and a pharmaceutically acceptable carrier for administration to a subject to stimulate a γδ T cell subpopulation.

19. A method for stimulating a γδ T cell subpopulation in vivo, comprising,
administering to a subject a unique TCR-γδ ligand, wherein the unique TCR-γδ ligand is a protein or a functional fragment thereof of Table 1 and a pharmaceutically acceptable carrier in an effective amount to stimulate a γδ T cell subpopulation.


SEQUENCE LISTING (1 sequence)

SEQ ID NO: 1 - protein, 6 aa, Artificial Sequence
  Synthetic
HHHHHH